(19) United States
(12) Patent Application Publication
(10) Pub. No.: US 2005/0288338 A1
(43) Pub. Date: Dec. 29, 2005

(54) Amido compounds and their use as pharmaceuticals

(76) Inventors: Wenqing Yao, Kennett Square, PA (US); Colin Zhang, Lansdale, PA (US); Meizhong Xu, Hockessin, DE (US); Jincong Zhuo, Boothwyn, PA (US); Chunhong He, Boothwyn, PA (US); Brian W. Metcalf, Moraga, CA (US)

Correspondence Address: COZEN O' CONNOR, P.C., 1900 MARKET STREET, PHILADELPHIA, PA, 19103-3508, US

(21) Appl. No.: 11/159,862

(22) Filed: Jun. 23, 2005

Related U.S. Application Data
(60) Provisional application No. 60/582,560, filed on Jun. 24, 2004.

(51) Int. Cl. A61K 31/44; A61K 31/445; A61K 31/165

(52) U.S. Cl. 514/357; 514/452; 514/618; 546/336; 549/355; 564/340; 514/426; 514/319

(57) ABSTRACT

The present invention relates to inhibitors of 11-β hydroxyl steroid dehydrogenase type 1, antagonists of the mineralocorticoid receptor MR, and pharmaceutical compositions thereof. The compounds of the invention can be useful in the treatment of various diseases associated with expression or activity of 11-β hydroxyl steroid dehydrogenase type 1 and/or diseases associated with aldosterone excess.

### CROSS REFERENCE TO RELATED APPLICATIONS

This application claims the benefit of U.S. Ser. No. 60/582,560, filed Jun. 24, 2004, the disclosure of which is incorporated herein by reference in its entirety.

### FIELD OF THE INVENTION

The present invention relates to modulators of 11-β hydroxyl steroid dehydrogenase type 1 (11βHSD1) and/or mineralocorticoid receptor (MR), compositions thereof and methods of using the same.

### BACKGROUND OF THE INVENTION

Glucocorticoids are steroid hormones that regulate fat metabolism, function and distribution. In vertebrates, glucocorticoids also have profound and diverse physiological effects on development, neurobiology, inflammation, blood pressure, metabolism and programmed cell death. In humans, the primary endogenously-produced glucocorticoid is cortisol. Cortisol is synthesized in the zona fasciculate of the adrenal cortex under the control of a short-term neuroendocrine feedback circuit called the hypothalamic-pituitary-adrenal (HPA) axis. Adrenal production of cortisol proceeds under the control of adrenocorticotrophic hormone (ACTH), a factor produced and secreted by the anterior pituitary. Production of ACTH in the anterior pituitary is itself highly regulated, driven by corticotropin releasing hormone (CRH) produced by the paraventricular nucleus of the hypothalamus. The HPA axis maintains circulating cortisol concentrations within restricted limits, with forward drive at the diurnal maximum or during periods of stress, and is rapidly attenuated by a negative feedback loop resulting from the ability of cortisol to suppress ACTH production in the anterior pituitary and CRH production in the hypothalamus.

Aldosterone is another hormone produced by the adrenal cortex; aldosterone regulates sodium and potassium homeostasis. Fifty years ago, a role for aldosterone excess in human disease was reported in a description of the syndrome of primary aldosteronism (Conn, (1955), J. Lab. Clin. Med. 45: 6-17). It is now clear that elevated levels of aldosterone are associated with deleterious effects on the heart and kidneys, and are a major contributing factor to morbidity and mortality in both heart failure and hypertension.

Two members of the nuclear hormone receptor superfamily, glucocorticoid receptor (GR) and mineralocorticoid receptor (MR), mediate cortisol function in vivo, while the primary intracellular receptor for aldosterone is the MR. These receptors are also referred to as ‘ligand-dependent transcription factors,’ because their functionality is dependent on the receptor being bound to its ligand (for example, cortisol); upon ligand-binding these receptors directly modulate transcription via DNA-binding zinc finger domains and transcriptional activation domains.

Historically, the major determinants of glucocorticoid action were attributed to three primary factors: 1) circulating levels of glucocorticoid (driven primarily by the HPA axis), 2) protein binding of glucocorticoids in circulation, and 3) intracellular receptor density inside target tissues. Recently, a fourth determinant of glucocorticoid function was identified: tissue-specific pre-receptor metabolism by glucocorticoid-activating and -inactivating enzymes. These 11-beta-hydroxysteroid dehydrogenase (11-β-HSD) enzymes act as pre-receptor control enzymes that modulate activation of the GR and MR by regulation of glucocorticoid hormones. To date, two distinct isozymes of 11-beta-HSD have been cloned and characterized: 11βHSD1 (also known as 11-beta-HSD type 1, 11betaHSD1, HSD11B1, HDL, and HSD11L) and 11βHSD2. 11βHSD1 and 11βHSD2 catalyze the interconversion of hormonally active cortisol (corticosterone in rodents) and inactive cortisone (11-dehydrocorticosterone in rodents). 11βHSD1 is widely distributed in rat and human tissues; expression of the enzyme and corresponding mRNA have been detected in lung, testis, and most abundantly in liver and adipose tissue. 11βHSD1 catalyzes both 11-beta-dehydrogenation and the reverse 11-oxoreduction reaction, although 11βHSD1 acts predominantly as a NADPH-dependent oxoreductase in intact cells and tissues, catalyzing the activation of cortisol from inert cortisone (Low et al. (1994) J. Mol. Endocrin. 13: 167-174) and has been reported to regulate glucocorticoid access to the GR. Conversely, 11βHSD2 expression is found mainly in mineralocorticoid target tissues such as kidney, placenta, colon and salivary gland, acts as an NAD-dependent dehydrogenase catalyzing the inactivation of cortisol to cortisone (Albiston et al. (1994) Mol. Cell. Endocrin. 105: R11-R17), and has been found to protect the MR from glucocorticoid excess, such as high levels of receptor-active cortisol (Blum, et al., (2003) Prog. Nucl. Acid Res. Mol. Biol. 75:173-216).

In vitro, the MR binds cortisol and aldosterone with equal affinity. The tissue specificity of aldosterone activity, however, is conferred by the expression of 11βHSD2 (Funder et al. (1988), Science 242: 583-585). The inactivation of cortisol to cortisone by 11βHSD2 at the site of the MR enables aldosterone to bind to this receptor in vivo. The binding of aldosterone to the MR results in dissociation of the ligand-activated MR from a multiprotein complex containing chaperone proteins, translocation of the MR into the nucleus, and its binding to hormone response elements in regulatory regions of target gene promoters. Within the distal nephron of the kidney, induction of serum and glucocorticoid inducible kinase-1 (sgk-1) expression leads to the absorption of Na⁺ ions and water through the epithelial sodium channel, as well as potassium excretion with subsequent volume expansion and hypertension (Bhargava et al., (2001), Endo 142: 1587-1594).

In humans, elevated aldosterone concentrations are associated with endothelial dysfunction, myocardial infarction, left ventricular atrophy, and death. In attempts to modulate these ill effects, multiple intervention strategies have been adopted to control aldosterone overactivity and attenuate the resultant hypertension and its associated cardiovascular consequences. Inhibition of angiotensin-converting enzyme (ACE) and blockade of the angiotensin type I receptor (ATIR) are two strategies that directly impact the rennin-angiotensin-aldosterone system (RAAS). However, although ACE inhibition and ATIR antagonism initially reduce aldosterone concentrations, circulating concentrations of this hormone return to baseline levels with chronic therapy (known as ‘aldosterone escape’). Importantly, co-administration of the MR antagonist Spironolactone or Eplerenone directly blocks the deleterious effects of this escape mechanism and dramatically reduces patient mortality (Pitt et al., New England J. Med. (1999), 341: 709-719; Pitt et al., New England J. Med. (2003), 348: 1309-1321). Therefore, MR antagonism may be an important treatment strategy for many patients with hypertension and cardiovascular disease, particularly those hypertensive patients at risk for target-organ damage.

Mutations in either of the genes encoding the 11-beta-HSD enzymes are associated with human pathology. For example, 11βHSD2 is expressed in aldosterone-sensitive tissues such as the distal nephron, salivary gland, and colonic mucosa where its cortisol dehydrogenase activity serves to protect the intrinsically non-selective MR from illicit occupation by cortisol (Edwards et al. (1988) Lancet 2: 986-989). Individuals with mutations in 11βHSD2 are deficient in this cortisol-inactivation activity and, as a result, present with a syndrome of apparent mineralocorticoid excess (also referred to as ‘SAME’) characterized by hypertension, hypokalemia, and sodium retention (Wilson et al. (1998) Proc. Natl. Acad. Sci. 95: 10200-10205). Likewise, mutations in 11βHSD1, a primary regulator of tissue-specific glucocorticoid bioavailability, and in the gene encoding a co-localized NADPH-generating enzyme, hexose 6-phosphate dehydrogenase (H6PD), can result in cortisone reductase deficiency (CRD), in which activation of cortisone to cortisol does not occur, resulting in adrenocorticotropin-mediated androgen excess. CRD patients excrete virtually all glucocorticoids as cortisone metabolites (tetrahydrocortisone) with low or absent cortisol metabolites (tetrahydrocortisols). When challenged with oral cortisone, CRD patients exhibit abnormally low plasma cortisol concentrations. These individuals present with ACTH-mediated androgen excess (hirsutism, menstrual irregularity, hyperandrogenism), a phenotype resembling polycystic ovary syndrome (PCOS) (Draper et al. (2003) Nat. Genet. 34: 434-439).

The importance of the HPA axis in controlling glucocorticoid excursions is evident from the fact that disruption of homeostasis in the HPA axis by either excess or deficient secretion or action results in Cushing's syndrome or Addison's disease, respectively (Miller and Chrousos (2001) Endocrinology and Metabolism, eds. Felig and Frohman (McGraw-Hill, New York), 4^{th} Ed.: 387-524). Patients with Cushing's syndrome (a rare disease characterized by systemic glucocorticoid excess originating from the adrenal or pituitary tumors) or receiving glucocorticoid therapy develop reversible visceral fat obesity. Interestingly, the phenotype of Cushing's syndrome patients closely resembles that of Reaven's metabolic syndrome (also known as Syndrome X or insulin resistance syndrome) the symptoms of which include visceral obesity, glucose intolerance, insulin resistance, hypertension, type 2 diabetes and hyperlipidemia (Reaven (1993) Ann. Rev. Med. 44: 121-131). However, the role of glucocorticoids in prevalent forms of human obesity has remained obscure because circulating glucocorticoid concentrations are not elevated in the majority of metabolic syndrome patients. In fact, glucocorticoid action on target tissue depends not only on circulating levels but also on intracellular concentration, locally enhanced action of glucocorticoids in adipose tissue and skeletal muscle has been demonstrated in metabolic syndrome. Evidence has accumulated that enzyme activity of 11βHSD1, which regenerates active glucocorticoids from inactive forms and plays a central role in regulating intracellular glucocorticoid concentration, is commonly elevated in fat depots from obese individuals. This suggests a role for local glucocorticoid reactivation in obesity and metabolic syndrome.

Given the ability of 11βHSD1 to regenerate cortisol from inert circulating cortisone, considerable attention has been given to its role in the amplification of glucocorticoid function. 11βHSD1 is expressed in many key GR-rich tissues, including tissues of considerable metabolic importance such as liver, adipose, and skeletal muscle, and, as such, has been postulated to aid in the tissue-specific potentiation of glucocorticoid-mediated antagonism of insulin function. Considering a) the phenotypic similarity between glucocorticoid excess (Cushing's syndrome) and the metabolic syndrome with normal circulating glucocorticoids in the latter, as well as b) the ability of 1βHSD1 to generate active cortisol from inactive cortisone in a tissue-specific manner, it has been suggested that central obesity and the associated metabolic complications in syndrome X result from increased activity of 11βHSD1 within adipose tissue, resulting in ‘Cushing's disease of the omentum’ (Bujalska et al. (1997) Lancet 349: 1210-1213). Indeed, 11βHSD1 has been shown to be upregulated in adipose tissue of obese rodents and humans (Livingstone et al. (2000) Endocrinology 131: 560-563; Rask et al. (2001) J. Clin. Endocrinol. Metab. 86: 1418-1421; Lindsay et al. (2003) J. Clin. Endocrinol. Metab. 88: 2738-2744; Wake et al. (2003) J. Clin. Endocrinol. Metab. 88: 3983-3988).

Additional support for this notion has come from studies in mouse transgenic models. Adipose-specific overexpression of 11βHSD1 under the control of the aP2 promoter in mouse produces a phenotype remarkably reminiscent of human metabolic syndrome (Masuzaki et al. (2001) Science 294: 2166-2170; Masuzaki et al. (2003) J. Clinical Invest. 112: 83-90). Importantly, this phenotype occurs without an increase in total circulating corticosterone, but rather is driven by a local production of corticosterone within the adipose depots. The increased activity of 11βHSD1 in these mice (2-3 fold) is very similar to that observed in human obesity (Rask et al. (2001) J. Clin. Endocrinol. Metab. 86: 1418-1421). This suggests that local 11βHSD1-mediated conversion of inert glucocorticoid to active glucocorticoid can have profound influences whole body insulin sensitivity.

Based on this data, it would be predicted that the loss of 11βHSD1 would lead to an increase in insulin sensitivity and glucose tolerance due to a tissue-specific deficiency in active glucocorticoid levels. This is, in fact, the case as shown in studies with 11βHSD1-deficient mice produced by homologous recombination (Kotelevstev et al. (1997) Proc. Natl. Acad. Sci. 94: 14924-14929; Morton et al. (2001) J. Biol. Chem. 276: 41293-41300; Morton et al. (2004) Diabetes 53: 931-938). These mice are completely devoid of 11-keto reductase activity, confirming that 11βHSD1 encodes the only activity capable of generating active corticosterone from inert 11-dehydrocorticosterone. 11 βHSD1-deficient mice are resistant to diet- and stress-induced hyperglycemia, exhibit attenuated induction of hepatic gluconeogenic enzymes (PEPCK, G6P), show increased insulin sensitivity within adipose, and have an improved lipid profile (decreased triglycerides and increased cardio-protective HDL). Additionally, these animals show resistance to high fat diet-induced obesity. Taken together, these transgenic mouse studies confirm a role for local reactivation of glucocorticoids in controlling hepatic and peripheral insulin sensitivity, and suggest that inhibition of 11βHSD1 activity may prove beneficial in treating a number of glucocorticoid-related disorders, including obesity, insulin resistance, hyperglycemia, and hyperlipidemia.

Data in support of this hypothesis has been published. Recently, it was reported that 11βHSD1 plays a role in the pathogenesis of central obesity and the appearance of the metabolic syndrome in humans. Increased expression of the 11βHSD1 gene is associated with metabolic abnormalities in obese women and that increased expression of this gene is suspected to contribute to the increased local conversion of cortisone to cortisol in adipose tissue of obese individuals (Engeli, et al., (2004) Obes. Res. 12: 9-17).

A new class of 11βHSD1 inhibitors, the arylsulfonamidothiazoles, was shown to improve hepatic insulin sensitivity and reduce blood glucose levels in hyperglycemic strains of mice (Barf et al. (2002) J. Med. Chem. 45: 3813-3815; Alberts et al. Endocrinology (2003) 144: 4755-4762). Furthermore, it was recently reported that selective inhibitors of 11βHSD1 can ameliorate severe hyperglycemia in genetically diabetic obese mice. Thus, 11βHSD1 is a promising pharmaceutical target for the treatment of the Metabolic Syndrome (Masuzaki, et al., (2003) Curr. Drug Targets Immune Endocr. Metabol. Disord. 3: 255-62).

A. Obesity and Metabolic Syndrome

As described above, multiple lines of evidence suggest that inhibition of 11βHSD1 activity can be effective in combating obesity and/or aspects of the metabolic syndrome cluster, including glucose intolerance, insulin resistance, hyperglycemia, hypertension, and/or hyperlipidemia. Glucocorticoids are known antagonists of insulin action, and reductions in local glucocorticoid levels by inhibition of intracellular cortisone to cortisol conversion should increase hepatic and/or peripheral insulin sensitivity and potentially reduce visceral adiposity. As described above, 11βHSD1 knockout mice are resistant to hyperglycemia, exhibit attenuated induction of key hepatic gluconeogenic enzymes, show markedly increased insulin sensitivity within adipose, and have an improved lipid profile. Additionally, these animals show resistance to high fat diet-induced obesity (Kotelevstev et al. (1997) Proc. Natl. Acad. Sci. 94: 14924-14929; Morton et al. (2001) J. Biol. Chem. 276: 41293-41300; Morton et al. (2004) Diabetes 53: 931-938). Thus, inhibition of 11βHSD1 is predicted to have multiple beneficial effects in the liver, adipose, and/or skeletal muscle, particularly related to alleviation of component(s) of the metabolic syndrome and/or obesity.

B. Pancreatic Function

Glucocorticoids are known to inhibit the glucose-stimulated secretion of insulin from pancreatic beta-cells (Billaudel and Sutter (1979) Horm. Metab. Res. 11: 555-560). In both Cushing's syndrome and diabetic Zucker falfa rats, glucose-stimulated insulin secretion is markedly reduced (Ogawa et al. (1992) J. Clin. Invest. 90: 497-504). 11βHSD1 mRNA and activity has been reported in the pancreatic islet cells of ob/ob mice and inhibition of this activity with carbenoxolone, an 11βHSD1 inhibitor, improves glucose-stimulated insulin release (Davani et al. (2000) J. Biol. Chem. 275: 34841-34844). Thus, inhibition of 11βHSD1 is predicted to have beneficial effects on the pancreas, including the enhancement of glucose-stimulated insulin release.

C. Cognition and Dementia

Mild cognitive impairment is a common feature of aging that may be ultimately related to the progression of dementia. In both aged animals and humans, inter-individual differences in general cognitive function have been linked to variability in the long-term exposure to glucocorticoids (Lupien et al. (1998) Nat. Neurosci. 1: 69-73). Further, dysregulation of the HPA axis resulting in chronic exposure to glucocorticoid excess in certain brain subregions has been proposed to contribute to the decline of cognitive function (McEwen and Sapolsky (1995) Curr. Opin. Neurobiol. 5: 205-216). 11βHSD1 is abundant in the brain, and is expressed in multiple subregions including the hippocampus, frontal cortex, and cerebellum (Sandeep et al. (2004) Proc. Natl. Acad. Sci. Early Edition: 1-6). Treatment of primary hippocampal cells with the 11βHSD1 inhibitor carbenoxolone protects the cells from glucocorticoid-mediated exacerbation of excitatory amino acid neurotoxicity (Rajan et al. (1996) J. Neurosci. 16: 65-70). Additionally, 11βHSD1-deficient mice are protected from glucocorticoid-associated hippocampal dysfunction that is associated with aging (Yau et al. (2001) Proc. Natl. Acad. Sci. 98: 4716-4721). In two randomized, double-blind, placebo-controlled crossover studies, administration of carbenoxolone improved verbal fluency and verbal memory (Sandeep et al. (2004) Proc. Natl. Acad. Sci. Early Edition: 1-6). Thus, inhibition of 11βHSD1 is predicted to reduce exposure to glucocorticoids in the brain and protect against deleterious glucocorticoid effects on neuronal function, including cognitive impairment, dementia, and/or depression.

D. Intra-Ocular Pressure

Glucocorticoids can be used topically and systemically for a wide range of conditions in clinical ophthalmology. One particular complication with these treatment regimens is corticosteroid-induced glaucoma. This pathology is characterized by a significant increase in intra-ocular pressure (IOP). In its most advanced and untreated form, IOP can lead to partial visual field loss and eventually blindness. IOP is produced by the relationship between aqueous humour production and drainage. Aqueous humour production occurs in the non-pigmented epithelial cells (NPE) and its drainage is through the cells of the trabecular meshwork. 11βHSD1 has been localized to NPE cells (Stokes et al. (2000) Invest. Ophthalmol. Vis. Sci. 41: 1629-1683; Rauz et al. (2001) Invest. Ophthalmol. Vis. Sci. 42: 2037-2042) and its function is likely relevant to the amplification of glucocorticoid activity within these cells. This notion has been confirmed by the observation that free cortisol concentration greatly exceeds that of cortisone in the aqueous humour (14:1 ratio). The functional significance of 11βHSD1 in the eye has been evaluated using the inhibitor carbenoxolone in healthy volunteers (Rauz et al. (2001) Invest. Ophthalmol. Vis. Sci. 42: 2037-2042). After seven days of carbenoxolone treatment, IOP was reduced by 18%. Thus, inhibition of 11βHSD1 in the eye is predicted to reduce local glucocorticoid concentrations and IOP, producing beneficial effects in the management of glaucoma and other visual disorders.

E. Hypertension

Adipocyte-derived hypertensive substances such as leptin and angiotensinogen have been proposed to be involved in the pathogenesis of obesity-related hypertension (Matsuzawa et al. (1999) Ann. N.Y. Acad. Sci. 892: 146-154; Wajchenberg (2000) Endocr. Rev. 21: 697-738). Leptin, which is secreted in excess in aP2-11βHSD1 transgenic mice (Masuzaki et al. (2003) J. Clinical Invest. 112: 83-90), can activate various sympathetic nervous system pathways, including those that regulate blood pressure (Matsuzawaetal. (1999) Ann. N.Y. Acad. Sci. 892: 146-154). Additionally, the renin-angiotensin system (RAS) has been shown to be a major determinant of blood pressure (Walker et al. (1979) Hypertension 1: 287-291). Angiotensinogen, which is produced in liver and adipose tissue, is the key substrate for renin and drives RAS activation. Plasma angiotensinogen levels are markedly elevated in aP2-11βHSD1 transgenic mice, as are angiotensin II and aldosterone (Masuzaki et al. (2003) J. Clinical Invest. 112: 83-90). These forces likely drive the elevated blood pressure observed in aP2-11βHSD1 transgenic mice. Treatment of these mice with low doses of an angiotensin II receptor antagonist abolishes this hypertension (Masuzaki et al. (2003) J. Clinical Invest. 112: 83-90). This data illustrates the importance of local glucocorticoid reactivation in adipose tissue and liver, and suggests that hypertension may be caused or exacerbated by 11βHSD1 activity. Thus, inhibition of 11βHSD1 and reduction in adipose and/or hepatic glucocorticoid levels is predicted to have beneficial effects on hypertension and hypertension-related cardiovascular disorders.

F. Bone Disease

Glucocorticoids can have adverse effects on skeletal tissues. Continued exposure to even moderate glucocorticoid doses can result in osteoporosis (Cannalis (1996) J. Clin. Endocrinol. Metab. 81: 3441-3447) and increased risk for fractures. Experiments in vitro confirm the deleterious effects of glucocorticoids on both bone-resorbing cells (also known as osteoclasts) and bone forming cells (osteoblasts). 11βHSD1 has been shown to be present in cultures of human primary osteoblasts as well as cells from adult bone, likely a mixture of osteoclasts and osteoblasts (Cooper et al. (2000) Bone 27: 375-381), and the 11βHSD1 inhibitor carbenoxolone has been shown to attenuate the negative effects of glucocorticoids on bone nodule formation (Bellows et al. (1998) Bone 23: 119-125). Thus, inhibition of 11βHSD1 is predicted to decrease the local glucocorticoid concentration within osteoblasts and osteoclasts, producing beneficial effects in various forms of bone disease, including osteoporosis.

Small molecule inhibitors of 11βHSD1 are currently being developed to treat or prevent 11βHSD1-related diseases such as those described above. For example, certain amide-based inhibitors are reported in WO 2004/089470, WO 2004/089896, WO 2004/056745, and WO 2004/065351.

Antagonists of 11βHSD1 have been evaluated in human clinical trials (Kurukulasuriya, et al., (2003) Curr. Med. Chem. 10: 123-53).

In light of the experimental data indicating a role for 11βHSD1 in glucocorticoid-related disorders, metabolic syndrome, hypertension, obesity, insulin resistance, hyperglycemia, hyperlipidemia, type 2 diabetes, androgen excess (hirsutism, menstrual irregularity, hyperandrogenism) and polycystic ovary syndrome (PCOS), therapeutic agents aimed at augmentation or suppression of these metabolic pathways, by modulating glucocorticoid signal transduction at the level of 11βHSD1 are desirable.

Furthermore, because the MR binds to aldosterone (its natural ligand) and cortisol with equal affinities, compounds that are designed to interact with the active site of 11βHSD1 (which binds to cortisone/cortisol) may also interact with the MR and act as antagonists. Because the MR is implicated in heart failure, hypertension, and related pathologies including atherosclerosis, arteriosclerosis, coronary artery disease, thrombosis, angina, peripheral vascular disease, vascular wall damage, and stroke, MR antagonists are desirable and may also be useful in treating complex cardiovascular, renal, and inflammatory pathologies including disorders of lipid metabolism including dyslipidemia or hyperlipoproteinaemia, diabetic dyslipidemia, mixed dyslipidemia, hypercholesterolemia, hypertriglyceridemia, as well as those associated with type 1 diabetes, type 2 diabetes, obesity, metabolic syndrome, and insulin resistance, and general aldosterone-related target-organ damage.

As evidenced herein, there is a continuing need for new and improved drugs that target 11βHSD1 and/or MR. The compounds, compositions and methods described herein help meet this and other needs.

### SUMMARY OF THE INVENTION

The present invention provides, inter alia, compounds of Formula 1: 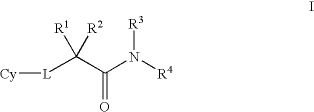
or pharmaceutically acceptable salts or prodrugs thereof, wherein constituent members are defined herein.

The present invention further provides compositions comprising compounds of the invention and a pharmaceutically acceptable carrier.

The present invention further provides methods of modulating 11βHSD1 or MR by contacting said 11βHSD1 or MR with a compound of the invention.

The present invention further provides methods of inhibiting 11βHSD1 or MR by contacting said 11βHSD1 or MR with a compound of the invention.

The present invention further provides methods of inhibiting the conversion of cortisone to cortisol in a cell by contacting the cell with a compound of the invention.

The present invention further provides methods of inhibiting the production of cortisol in a cell by contacting the cell with a compound of the invention.

The present invention further provides methods of increasing insulin sensitivity in a cell.

The present invention further provides methods of treating diseases associated with activity or expression of 11βHSD1 or MR.

### DETAILED DESCRIPTION

In a first aspect, the present invention provides, inter alia, compounds of Formula I: 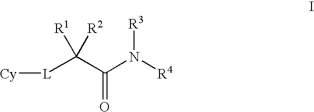
or pharmaceutically acceptable salt or prodrug thereof, wherein:

Cy is aryl, heteroaryl, cycloalkyl or heterocycloalkyl, each optionally substituted by 1, 2, 3, 4 or 5-W—X—Y-Z;

L is SO₂, (CR⁶R⁷)ₙO(CR⁶R⁷)ₚ or (CR⁶R⁷)ₙS(CR⁶R⁷)ₚ;

R¹ and R² together with the C atom to which they are attached form a 3-, 4-, 5-, 6- or 7-membered cycloalkyl group or a 3-, 4-, 5-, 6- or 7-membered heterocycloalkyl group, each optionally substituted by 1, 2 or 3 R⁵;

R³ is H, C₁₋₆alkyl, cycloalkyl, heterocycloalkyl, arylalkyl, cycloalkylalkyl, heteroarylalkyl, or heterocycloalkylalkyl;

R⁴ is C₁₋₆alkyl, cycloalkyl, heterocycloalkyl, arylalkyl, cycloalkylalkyl, heteroarylalkyl, heterocycloalkylalkyl, each optionally substituted by 1, 2 or 3-W′—X′—Y′-Z′;

R⁵ is halo, C₁₋₄ alkyl, C₁₋₄ haloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, or NR^{c}C(O)OR^{a};

R⁶ and R⁷ are each, independently, H, halo, C₁₋₄ alkyl, C₁₋₄ haloalkyl, aryl, cycloalkyl, heteroaryl, heterocycloalkyl, CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}, NR^{c′}R^{d′}, NR^{c′}C(O)R^{d′}, NR^{c′}C(O)OR^{a′}, S(O)R^{b′}, S(O)NR^{c′}R^{d′}, S(O)₂R^{b′}, or S(O)₂NR^{c′}R^{d′};

W, W′ and W″ are each, independently, absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, or NR^{e}CONR^{f}, wherein said C₁₋₆ alkylenyl, C₂₋₆ alkenylenyl, C₂₋₆ alkynylenyl are each optionally substituted by 1, 2 or 3 halo, OH, C₁₋₄ alkoxy, C₁₋₄ haloalkoxy, amino, C₁₋₄ alkylamino or C₂₋₈dialkylamino;

X, X′ and X″ are each, independently, absent, C₁₋₆ alkylenyl, C₂₋₆ alkenylenyl, C₂₋₆ alkynylenyl, aryl, cycloalkyl, heteroaryl or heterocycloalkyl, wherein said C₁₋₆ alkylenyl, C₂₋₆ alkenylenyl, C₂₋₆ alkynylenyl, cycloalkyl, heteroaryl or heterocycloalkyl is optionally substituted by one or more halo, CN, NO₂, OH, C₁₋₄ alkoxy, C₁₋₄ haloalkoxy, amino, C₁₋₄ alkylamino or C₂₋₈ dialkylamino;

Y, Y′ and Y″ are each, independently, absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, or NR^{e}CONR^{f}, wherein said C₁₋₆ alkylenyl, C₂₋₆ alkenylenyl, C₂₋₆ alkynylenyl are each optionally substituted by 1, 2 or 3 halo, OH, C₁₋₄ alkoxy, C₁₋₄ haloalkoxy, amino, C₁₋₄ alkylamino or C₂₋₈ dialkylamino;
Z, Z′ and Z″ are each, independently, H, halo, CN, NO₂, OH, C₁₋₄ alkoxy, C₁₋₄ haloalkoxy, amino, C₁₋₄ alkylamino or C₂₋₈ dialkylamino, C₁₋₆ alkyl, C₂₋₆ alkenyl, C₂₋₆ alkynyl, aryl, cycloalkyl, heteroaryl or heterocycloalkyl, wherein said C₁₋₆ alkyl, C₂₋₆ alkenyl, C₂₋₆ alkynyl, aryl, cycloalkyl, heteroaryl or heterocycloalkyl is optionally substituted by 1, 2 or 3 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, S(O)R^{b}, S(O)NR^{c}R^{d}, S(O)₂R^{b}, or S(O)₂NR^{c}R^{d};

wherein two —W—X—Y-Z attached to the same atom, together with the atom to which they are attached, optionally form a 3-20 membered cycloalkyl or heterocycloalkyl group each optionally substituted by 1, 2 or 3-W″—X″—Y″-Z″;

or wherein two —W—X—Y-Z together with the carbon atom to which they are both attached optionally form a carbonyl;

or wherein two —W—X—Y-Z together with two adjacent atoms to which they are attached optionally form a 3-20 membered fused cycloalkyl group or 3-20 membered fused heterocycloalkyl group, each optionally substituted by 1, 2 or 3-W″—X″—Y″-Z″;

wherein two —W′—X′—Y′-Z′ together with the atom to which they are both attached optionally form a 3-20 membered cycloalkyl group or 3-20 membered heterocycloalkyl group, each optionally substituted by 1, 2 or 3-W″—X″—Y″-Z″;

or wherein two —W′—X′—Y′-Z′ together with the carbon atom to which they are both attached optionally form a carbonyl;

or wherein two —W′—X′—Y′-Z′ together with two adjacent atoms to which they are attached optionally form a 3-20 membered fused cycloalkyl group or 3-20 membered fused heterocycloalkyl group, each optionally substituted by 1, 2 or 3-W″—X″—Y″-Z″;

or wherein two —W′—X′—Y′-Z′ together with two adjacent atoms to which they are attached optionally form a 5- or 6-membered fused aryl or 5- or 6-membered fused heteroaryl group, each optionally substituted by 1, 2 or 3-W″—X″—Y″-Z″;

wherein —W—X—Y-Z is other than H;

wherein —W′—X′—Y′-Z′ is other than H;

wherein —W″—X″—Y″-Z″ is other than H;

R^{a} and R^{a′} are each, independently, H, C₁₋₆ alkyl, C₁₋₆ haloalkyl, C₂₋₆ alkenyl, C₂₋₆ alkynyl, aryl, cycloalkyl, heteroaryl or heterocycloalkyl;

R^{b} and R^{b′} are each, independently, H, C₁₋₆ alkyl, C₁₋₆ haloalkyl, C₂₋₆ alkenyl, C₂₋₆ alkynyl, aryl, cycloalkyl, heteroaryl or heterocycloalkyl;

R^{c} and R^{d} are each, independently, H, C₁₋₆ alkyl, C₁₋₆ haloalkyl, C₂₋₆ alkenyl, C₂₋₆ alkynyl, aryl, cycloalkyl, arylalkyl, or cycloalkylalkyl;

or R^{c} and R^{d} together with the N atom to which they are attached form a 4-, 5-, 6- or 7-membered heterocycloalkyl group;

R^{c′} and R^{d′} are each, independently, H, C₁₋₆ alkyl, C₁₋₆ haloalkyl, C₂₋₆ alkenyl, C₂₋₆ alkynyl, aryl, cycloalkyl, arylalkyl, or cycloalkylalkyl;

or R^{c′} and R^{d′} together with the N atom to which they are attached form a 4-, 5-, 6- or 7-membered heterocycloalkyl group;

R^{e} and R^{f} are each, independently, H, C₁₋₆ alkyl, C₁₋₆ haloalkyl, C₂₋₆ alkenyl, C₂₋₆ alkynyl, aryl, cycloalkyl, arylalkyl, or cycloalkylalkyl;

or R^{e} and R^{f} together with the N atom to which they are attached form a 4-, 5-, 6- or 7-membered heterocycloalkyl group;

n is 0, 1, 2 or 3; and

p is 0, 1, 2 or 3.

In some embodiments of the first aspect of the invention, when R³ is C₁₋₆ alkyl, R⁴ is other than C₁₋₆ alkyl.

In some embodiments of the first aspect of the invention, when L is SCH₂ and R³ is H, then R⁴ is other than 4-benzyloxycarbonyl-6-oxo-1,3,4,7,8,12b-hexahydro-2H-benzo[c]pyrido[1,2-a]azepin-7-yl.

In some embodiments of the first aspect of the invention, Cy is aryl or heteroaryl, each optionally substituted by 1, 2, 3, 4 or 5-W—X—Y-Z.

In some embodiments of the first aspect of the invention, Cy is aryl optionally substituted by 1, 2, 3, 4 or 5-W—X—Y-Z.

In some embodiments of the first aspect of the invention, Cy is phenyl optionally substituted by 1, 2, 3, 4 or 5-W—X—Y-Z.

In some embodiments of the first aspect of the invention, Cy is phenyl optionally substituted by 1, 2, 3, 4 or 5 halo.

In some embodiments of the first aspect of the invention, L is OCH₂.

In some embodiments of the first aspect of the invention, L is S or SCH₂.

In some embodiments of the first aspect of the invention, L is S.

In some embodiments of the first aspect of the invention, L is SCH₂.

In some embodiments of the first aspect of the invention, R¹ and R² together with the C atom to which they are attached form cyclopropyl optionally substituted by 1, 2 or 3 R⁵.

In some embodiments of the first aspect of the invention, R¹ and R² together with the C atom to which they are attached form cyclopropyl.

In some embodiments of the first aspect of the invention, R³ is H, C₁₋₆alkyl, cyclopropyl, cyclobutyl, cyclopentyl, cyclohexyl, cycloheptyl, indanyl, 1,2,3,4-tetrahydro-naphthyl, bicyclo[2.2.1]heptanyl, piperidinyl, piperazinyl, pyrrolidinyl, tetrahydrofuranyl, dihydro-furan-2-on-yl, cyclopropylethyl, cyclopropylpropyl, cyclohexylethyl, cyclohexylpropyl, cyclohexylbutyl, phenylpropyl, phenylbutyl, 2,3-dihydro-benzo[1,4]dioxinylmethyl, 1H-indolylethyl, 1H-indolylpropyl or 1H-indolylbutyl, each optionally substituted by 1, 2 or 3-W′—X′—Y′-Z′.

In some embodiments of the first aspect of the invention, R³ is H or cyclopropyl, cyclopentyl, or cyclohexyl.

In some embodiments of the first aspect of the invention, R³ is H or cyclopropyl.

In some embodiments of the first aspect of the invention, R³ is H.

In some embodiments of the first aspect of the invention, R⁴ is C₁₋₆ alkyl, cyclopropyl, cyclobutyl, cyclopentyl, cyclohexyl, cycloheptyl, indanyl, adamantyl, 1,2,3,4-tetrahydro-naphthyl, bicyclo[2.2.1]heptanyl (norbornyl), piperidinyl, piperazinyl, pyrrolidinyl, tetrahydrofuranyl, dihydro-furan-2-on-yl, tetrahydropyranyl, cyclopropylethyl, cyclopropylpropyl, cyclohexylmethyl, cyclohexylethyl, cyclohexylpropyl, cyclohexylbutyl, phenylethyl, phenylpropyl, phenylbutyl, 2,3-dihydro-benzo[1,4]dioxinylmethyl, pyridinylmethyl, pyridinylethyl, 1H-indolylethyl, 1H-indolylpropyl or 1H-indolylbutyl, each optionally substituted by 1, 2 or 3-W′—X′—Y′-Z′.

In some embodiments of the first aspect of the invention, -W—X—Y-Z is halo, C₁₋₄ alkyl, C₁₋₄ haloalkyl, OH. C₁₋₄ alkoxy, C₁₋₄ haloalkoxy, (alkoxy)-CO-cycloalkyl, (alkoxy)-CO-heterocycloalkyl, hydroxyalkyl, alkoxyalkyl, cycloalkyl, heterocycloalkyl, aryl, heteroaryl, arylalkyl or heteroarylalkyl.

In some embodiments of the first aspect of the invention, —W—X—Y-Z is halo, heteroaryl, or heterocycloalkyl.

In some embodiments of the first aspect of the invention, —W—X—Y-Z is halo.

In some embodiments of the first aspect of the invention, —W′—X′—Y′-Z′ is halo, C₁₋₄ alkyl, C₁₋₄ haloalkyl, OH, C₁₋₄ alkoxy, C₁₋₄ haloalkoxy, hydroxyalkyl, alkoxyalkyl, —COO-alkyl, aryl, heteroaryl, aryloxy, heteroaryloxy, arylalkyloxy, heteroarylalkyloxy, optionally substituted arylsulfonyl, optionally substituted heteroarylsulfonyl, aryl substituted by halo, heteroaryl substituted by halo.

In some embodiments of the first aspect of the invention, —W″—X″—Y″-Z″ is halo, cyano, C₁₋₄ cyanoalkyl, nitro, C₁₋₈ alkyl, C₁₋₈ alkenyl, C₁₋₈ haloalkyl, C₁₀₋ alkoxy, C₁₋₄ haloalkoxy, OH, C₁₋₈ alkoxyalkyl, amino, C₁₋₄ alkylamino, C₂₋₈ dialkylamino, OC(O)NR^{c}R^{d}, NR^{c}C(O)R^{d}, NR^{c}C(═NCN)NR^{d}, NR^{c}C(O)OR^{a}, aryloxy, heteroaryloxy, arylalkyloxy, heteroarylalkyloxy, heteroaryloxyalkyl, aryloxyalkyl, aryl, heteroaryl, cycloalkyl, heterocycloalkyl, arylalkyl, arylalkenyl, arylalkynyl, heteroarylalkyl, heteroarylalkenyl, heteroarylalkynyl, cycloalkylalkyl, or heterocycloalkylalkyl;

wherein each of said C₁₋₈ alkyl, C₁₋₈ alkenyl, C₁₋₈ haloalkyl, C₁₋₈ alkoxy, aryloxy, heteroaryloxy, arylalkyloxy, heteroarylalkyloxy, heteroaryloxyalkyl, aryloxyalkyl, aryl, heteroaryl, cycloalkyl, heterocycloalkyl, arylalkyl, arylalkenyl, arylalkynyl, heteroarylalkyl, heteroarylalkenyl, heteroarylalkynyl, cycloalkylalkyl, or heterocycloalkylalkyl is optionally substituted by 1, 2, or 3 halo, cyano, nitro, hydroxyl-(C₁₋₆ alkyl), aminoalkyl, dialkylaminoalkyl, C₁₋₄ alkyl, C₁₋₄ haloalkyl, C₁₋₄ alkoxy, C₁₋₄ haloalkoxy, OH, C₁₋₈ alkoxyalkyl, amino, C₁₋₄ alkylamino, C₂₋₈ dialkylamino, C(O)NR^{c}R^{d}, C(O)OR^{a}, NR^{c}C(O)R^{d}, NR^{c}S(O)₂R^{d}, (C₁₋₄ alkyl)sulfonyl, arylsulfonyl, aryl, heteroaryl, cycloalkyl, or heterocycloalkyl.

In some embodiments of the first aspect of the invention, —W″—X″—Y″-Z″ is halo, cyano, C₁₋₄ cyanoalkyl, nitro, C₁₋₄ nitroalkyl, C₁₋₄ alkyl, C₁₋₄ haloalkyl, C₁₋₄ alkoxy, C₁₋₄ haloalkoxy, OH, C₁₋₈ alkoxyalkyl, amino, C₁₋₄ alkylamino, C₂₋₈ dialkylamino, aryl, heteroaryl, cycloalkyl, heterocycloalkyl, arylalkyl, heteroarylalkyl, cycloalkylalkyl, or heterocycloalkylalkyl.

In a second aspect, the present invention provides, inter alia, compounds of Formula I: 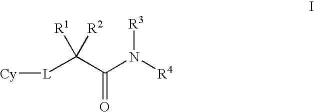
or pharmaceutically acceptable salt or prodrug thereof, wherein:

Cy is phenyl or heteroaryl, each optionally substituted by 1, 2, 3, 4 or 5 R^{1a};

L is absent or (CR⁶R⁷)ₘ;

R¹ and R² together with the carbon atom to which they are attached form cyclopropyl or cyclobutyl;

R³ is H, C₁₋₆ alkyl, cycloalkyl, heterocycloalkyl, or cycloalkylalkyl;

R⁴ is cyclopropyl, (CR^{4a}R^{4b})ₙCy², (CR^{4a}R^{4b})ₜCy³, (CHR^{4c})Cy³, (CR^{4a}R^{4b})ₜ₁Cy⁴, (CR^{4a}R^{4b})ₜCH₂OH, (CR^{4a}R^{4b})ₜ—O-phenyl, —CR^{6a}R^{7a}R^{8a}, or (CH₂)ₜCy⁵, wherein said cyclopropyl is optionally substituted by 1, 2 or 3 halo, C₁₋₃ alkyl, C₁₋₃ haloalkyl, phenyl, benzyl, C(O)OR^{10a} or OR^{10a};

R⁶ and R⁷ are each, independently, H, halo, C₁₋₄ alkyl, C₁₋₄ haloalkyl, aryl, cycloalkyl, heteroaryl, heterocycloalkyl, CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}, NR^{c′}R^{d′}, NR^{c′}C(O)R^{d′}, NR^{c′}C(O)OR^{a′}, S(O)R^{b′}, S(O)NR^{c′}R^{d′}, S(O)₂R^{b′}, or S(O)₂NR^{c′}R^{d′};

R^{1a} and R^{1b} are each, independently, halo, CN, NO₂, OH, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, S(O)R^{b}, S(O)NR^{c}R^{d}, S(O)₂R^{b}, S(O)₂NR^{c}R^{d}, C₁₋₄ alkoxy, C₁₋₄ haloalkoxy, amino, C₁₋₄ alkylamino, C₂₋₈ dialkylamino, C₁₋₆ alkenyl, C₂₋₆ alkynyl, aryl, arylsulfonyl, cycloalkyl, heteroaryl or heterocycloalkyl, wherein said C₁₋₆ alkyl, C₂₋₆ alkenyl, C₂₋₆ alkynyl, arylsulfonyl, aryl, cycloalkyl, heteroaryl or heterocycloalkyl is optionally substituted by 1, 2 or 3 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, S(O)R^{b}, S(O)NR^{c}R^{d}, S(O)₂R^{b}, or S(O)₂NR^{c}R^{d};

R^{4a} and R^{4b} are each, independently, H, halo, OH, CN, C₁₋₄alkyl, C₁₋₄ alkoxy, wherein said C₁₋₄ alkyl or C₁₋₄ alkoxy is optionally substituted with one or more halo, CN, NO₂, OH, C₁₋₄ alkoxy, C₁₋₄ haloalkoxy, amino, C₁₋₄ alkylamino or C₂₋₈ dialkylamino;

R^{4c} is OH, CN, C₁₋₄ alkyl, C₁₋₄ alkoxy, wherein said C₁₋₄ alkyl or C₁₋₄ alkoxy is optionally substituted with one or more halo, CN, NO₂, OH, C₁₋₄ alkoxy, C₁₋₄ haloalkoxy, amino, C₁₋₄ alkylamino or C₂₋₈ dialkylamino;

R^{5a} and R^{5b} are each, independently, H, halo, OH, CN, C₁₋₄ alkyl, C₁₋₄ alkoxy, wherein said C₁₋₄ alkyl or C₁₋₄ alkoxy is optionally substituted with one or more halo, CN, NO₂, OH, C₁₋₄ alkoxy, C₁₋₄ haloalkoxy, amino, C₁₋₄ alkylamino or C₂₋₈ dialkylamino;

R^{6a} is H or methyl;

R^{7a} is methyl or CH₂OH;

R^{8a} is C₂₋₆ alkyl or —(CR^{5a}R^{5b})ₚR^{9a}, wherein said C₂₋₆ alkyl is optionally substituted with one or more halo, CN, NO₂, OH, C₁₋₄ alkoxy or C₁₋₄ haloalkoxy;

R^{9a} is halo, CN, NO₂, OH, C₁₋₄ alkoxy, C₁₋₄ haloalkoxy, amino, C₁₋₄ alkylamino, C₂₋₈ dialkylamino, OR^{10b}, SR^{10b}, C(O)R^{10b}, C(O)NR^{10b}R^{11b}, C(O)OR^{10b}, OC(O)R^{10b}, OC(O)NR^{10b}R^{11b}, NR^{10b}R^{11b}, NR^{10b}C(O)R^{11b}, NR^{10b}C(O)OR^{11b}, S(O)R¹⁰b, S(O)NR^{10b}R^{11b}, S(O)₂R^{11b}, S(O)₂NR^{10b}R^{11b}, cycloalkyl, aryl, heteroaryl, wherein said cycloalkyl, aryl or heteroaryl is optionally substituted by one or more halo, C₁₋₄ alkyl, C₁₋₄ haloalkyl, CN, NO₂, OH, C₁₋₄ alkoxy, C₁₋₄ haloalkoxy, amino, C₁₋₄ alkylamino or C₂₋₈ dialkylamino;

R^{10a} is H, C₁₋₆ alkyl, C₁₋₆ haloalkyl, C₂₋₆ alkenyl, C₂₋₆ alkynyl, aryl, cycloalkyl, heteroaryl or heterocycloalkyl;

R^{10b} and R^{11b} are each, independently, H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, cycloalkylalkyl, heteroarylalkyl or heterocycloalkylalkyl;

or R^{10b} and R^{11b} together with the N atom to which they are attached form a 4-, 5-, 6- or 7-membered heterocycloalkyl group;

Cy² is:

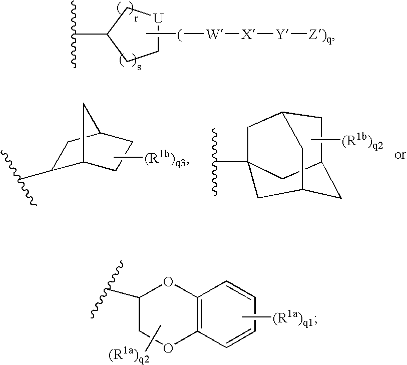

Cy³ is phenyl optionally substituted by 1, 2, 3, 4 or 5 R^{1b};

Cy⁴ is pyridinyl optionally substituted by 1, 2, 3, 4 or 5 R^{1b};

Cy⁵ is phenyl optionally substituted by 1, 2, 3, 4 or 5 halo or OH;

U is CH₂, NH, or O;

W′ and W″ are each, independently, absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, or NR^{e}CONR^{f}, wherein said C₁₋₆ alkylenyl, C₂₋₆ alkenylenyl, C₂₋₆ alkynylenyl are each optionally substituted by 1, 2 or 3 halo, OH, C₁₋₄ alkoxy, C₁₋₄ haloalkoxy, amino, C₁₋₄ alkylamino or C₂₋₈ dialkylamino;

X′ and X″ are each, independently, absent, C₁₋₆ alkylenyl, C₂₋₆ alkenylenyl, C₂₋₆ alkynylenyl, aryl, cycloalkyl, heteroaryl or heterocycloalkyl, wherein said C₁₋₆ alkylenyl, C₂₋₆ alkenylenyl, C₂₋₆ alkynylenyl, cycloalkyl, heteroaryl or heterocycloalkyl is optionally substituted by one or more halo, CN, NO₂, OH, C₁₋₄ alkoxy, C₁₋₄ haloalkoxy, amino, C₁₋₄ alkylamino or C₂₋₈ dialkylamino;

Y′ and Y″ are each, independently, absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, or NR^{e}CONR^{f}, wherein said C₁₋₆ alkylenyl, C₂₋₆ alkenylenyl, C₂₋₆ alkynylenyl are each optionally substituted by 1, 2 or 3 halo, OH, C₁₋₄ alkoxy, C₁₋₄ haloalkoxy, amino, C₁₋₄ alkylamino or C₂₋₈ dialkylamino;

Z′ and Z″ are each, independently, H, halo, CN, NO₂, OH, C₁₋₄ alkoxy, C₁₋₄ haloalkoxy, amino, C₁₋₄ alkylamino or C₂₋₈ dialkylamino, C₁₋₆ alkyl, C₂₋₆ alkenyl, C₂₋₆ alkynyl, aryl heteroaryl or heterocycloalkyl, wherein said C₁₋₆ alkyl, C₂₋₆ alkenyl, C₂₋₆ alkynyl, aryl, cycloalkyl, heteroaryl or heterocycloalkyl is optionally substituted by 1, 2 or 3 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, S(O)R^{b}, S(O)NR^{c}R^{d}, S(O)₂R^{b}, or S(O)₂NR^{c}R^{d};

wherein two —W′—X′—Y′-Z′ together with the atom to which they are both attached optionally form a 3-20 membered cycloalkyl group or 3-20 membered heterocycloalkyl group, each optionally substituted by 1, 2 or 3-W″—X″—Y″-Z″;

or wherein two —W′—X′—Y′-Z′ together with the carbon atom to which they are both attached optionally form a carbonyl;

wherein two —W′—X′—Y′-Z′ together with two adjacent atoms to which they are attached optionally form a 3-20 membered fused cycloalkyl group or 3-20 membered fused heterocycloalkyl group, each optionally substituted by 1, 2 or 3-W″—X″—Y″-Z″;

or wherein two —W′—X′—Y′-Z′ together with two adjacent atoms to which they are attached optionally form a 5- or 6-membered fused aryl or 5- or 6-membered fused heteroaryl group, each optionally substituted by 1, 2 or 3-W″—X″—Y″-Z″;

wherein —W′—X′—Y′-Z′ is other than H;

wherein —W″—X″—Y″-Z″ is other than H;

R^{a} and R^{a′} are each, independently, H, C₁₋₆ alkyl, C₁₋₆ haloalkyl, C₂₋₆ alkenyl, C₂₋₆ alkynyl, aryl, cycloalkyl, heteroaryl or heterocycloalkyl;

R^{b} and R^{b′} are each, independently, H, C₁₋₆ alkyl, C₁₋₆ haloalkyl, C₂₋₆ alkenyl, C₂₋₆ alkynyl, aryl, cycloalkyl, heteroaryl or heterocycloalkyl;

R^{c} and R^{d} are each, independently, H, C₁₋₆ alkyl, C₁₋₆ haloalkyl, C₂₋₆ alkenyl, C₂₋₆ alkynyl, aryl, cycloalkyl, arylalkyl, or cycloalkylalkyl;

or R^{c} and R^{d} together with the N atom to which they are attached form a 4-, 5-, 6- or 7-membered heterocycloalkyl group;

R^{e′} and R^{d′} are each, independently, H, C₁₋₆ alkyl, C₁₋₆ haloalkyl, C₂₋₆ alkenyl, C₂₋₆ alkynyl, aryl, cycloalkyl, arylalkyl, or cycloalkylalkyl;

or R^{c′} and R^{d′} together with the N atom to which they are attached form a 4-, 5-, 6- or 7-membered heterocycloalkyl group;

R^{e} and R^{f} are each, independently, H, C₁₋₆ alkyl, C₁₋₆ haloalkyl, C₂₋₆ alkenyl, C₂₋₆ alkynyl, aryl cycloalkyl, arylalkyl, or cycloalkylalkyl;

or R^{e} and R^{f} together with the N atom to which they are attached form a 4-, 5-, 6- or 7-membered heterocycloalkyl group;

m is 1, 2, 3 or 4;

n is 0, 1, 2, or 3;

t1 is 1, 2, or 3;

s is 1 or 2;

t is 2 or 3;

p is 1, 2, 3, 4 or 5;

q1 is 0, 1, 2, 3 or 4;

q2 is 0, 1, 2 or 3;

q3 is 1, 2 or 3;

q is 0, 1, 2, 3, 4 or 5; and

r is 1 or 2.

In some embodiments of the second aspect of the invention, when L is absent and R⁴ is (CR^{4a}R^{4b})ₜCy³, then at least one of R^{4a} and R^{4b} is other than H;

In some embodiments of the second aspect of the invention, when L is absent, R⁴ is (CR^{4a}R^{4b})ₙCy², and n is 0, then Cy² is other than unsubstituted cyclopentyl, 2-methylcyclohexyl, 4-[(7-chlorquinolin-4-yl)amino]cyclohexyl, 3-(9-chloro-3-methyl-4-oxoisoxazolo[4,3-c]quinolin-5(4H)-yl)cyclohexyl, 1-[3-(2-methoxyphenoxy)benzyl]-piperidin-4-yl, 1-[3-(2-methoxyphenoxy)benzyl]-pyrrolidin-3-yl, or 1,7,7-trimethylbicyclo[2.2.1]hept-2-yl;

In some embodiments of the second aspect of the invention, when L is absent, R⁴ is (CR^{4a}R^{4b})ₙCy² and n is 1, then Cy² is other than 1,3,4,6,7,11b-hexahydro-9-methoxy-2H-benzo[a]quinolizin-2-yl;

In some embodiments of the second aspect of the invention, when L is absent, R⁴ is (CR^{4a}R^{4b})ₙCy² and Cy² is unsubstituted admantyl, then Cy is other than phenyl;

In some embodiments of the second aspect of the invention, when L is absent, R⁴ is (CHR^{4c})Cy³ and R^{4c} is methyl, then Cy is other than unsubstituted phenyl; and

In some embodiments of the second aspect of the invention, when L is absent, R⁴ is (CR^{4a}R^{4b})ₜ₁Cy⁴ and t1 is 1, then then Cy is other than unsubstituted phenyl.

In some embodiments of the second aspect of the invention, L is absent.

In some embodiments of the second aspect of the invention, Cy is phenyl optionally substituted by 1, 2, 3, 4 or 5 R^{1a}.

In some embodiments of the second aspect of the invention, R¹ and R² together with the carbon atom to which they are attached form cyclopropyl.

In some embodiments of the second aspect of the invention, R^{1a} is halo, C₁₋₄ alkoxy, heterocycloalkyl, or heteroaryl, wherein said heterocycloalkyl or heteroaryl is optionally substituted by 1, 2 or 3 C(O)OR^{a}, CONR^{c}R^{d}, or COR^{b}.

In some embodiments of the second aspect of the invention, R^{1a} is halo or C₁₋₄ alkoxy

In some embodiments of the second aspect of the invention, R³ is H or C₁₋₆ alkyl

In some embodiments of the second aspect of the invention, R⁴ is (CR^{4a}R^{4b})ₙCy²

In some embodiments of the second aspect of the invention, R⁴ is (CR^{4a}R^{4b})ₙCy² and n is 0 or 1.

In some embodiments of the second aspect of the invention, R⁴ is (CR^{4a}R^{4b})ₙCy² and n is 1.

In some embodiments of the second aspect of the invention, R⁴ is

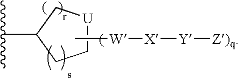

In some embodiments of the second aspect of the invention, U is CH₂, wherein said CH₂ is optionally substituted by —W″—X″—Y″-Z″.

In some embodiments of the second aspect of the invention, U is NH or O, wherein said NH is optionally substituted by —W″—X″—Y″-Z″.

In some embodiments of the second aspect of the invention, U is N(—W″—X″—Y″-Z″).

In some embodiments of the second aspect of the invention, R⁴ is cyclohexyl.

In some embodiments of the second aspect of the invention, —W′—X′—Y′-Z′ is halo, C₁₋₄ alkyl, C₁₋₄ haloalkyl, OH, C₁₋₄ alkoxy, C₁₋₄ haloalkoxy, hydroxyalkyl, alkoxyalkyl, —COO-alkyl, aryl, heteroaryl, aryloxy, heteroaryloxy, arylalkyloxy, heteroarylalkyloxy, optionally substituted arylsulfonyl, optionally substituted heteroarylsulfonyl, aryl substituted by halo, heteroaryl substituted by halo.

In some embodiments of the second aspect of the invention, —W″—X″—Y″-Z″ is halo, cyano, C₁₋₄ cyanoalkyl, nitro, C₁₋₈ alkyl, C₁₋₈ alkenyl, C₁₋₈ haloalkyl, C₁₀₋ alkoxy, C₁₋₄ haloalkoxy, OH, C₁₋₈ alkoxyalkyl, amino, C₁₋₄ alkylamino, C₂₋₈ dialkylamino, OC(O)NR^{c}R^{d}, NR^{c}C(O)R^{d}, NR^{c}C(═NCN)NR^{d}, NR^{c}C(O)OR^{a}, aryloxy, heteroaryloxy, arylalkyloxy, heteroarylalkyloxy, heteroaryloxyalkyl, aryloxyalkyl, aryl, heteroaryl, cycloalkyl, heterocycloalkyl, arylalkyl, arylalkenyl, arylalkynyl, heteroarylalkyl, heteroarylalkenyl, heteroarylalkynyl, cycloalkylalkyl, or heterocycloalkylalkyl;

wherein each of said C₁₋₈ alkyl, C₁₋₈ alkenyl, C₁₋₈ haloalkyl, C₁₋₈ alkoxy, aryloxy, heteroaryloxy, arylalkyloxy, heteroarylalkyloxy, heteroaryloxyalkyl, aryloxyalkyl, aryl, heteroaryl, cycloalkyl, heterocycloalkyl, arylalkyl, arylalkenyl, arylalkynyl, heteroarylalkyl, heteroarylalkenyl, heteroarylalkynyl, cycloalkylalkyl, or heterocycloalkylalkyl is optionally substituted by 1, 2, or 3 halo, cyano, nitro, hydroxyl-(C₁₋₆alkyl), aminoalkyl, dialkylaminoalkyl, C₁₋₄ alkyl, C₁₋₄ haloalkyl, C₁₋₄ alkoxy, C₁₋₄ haloalkoxy, OH, C₁₋₈ alkoxyalkyl, amino, C₁₋₄ alkylamino, C₂₋₈ dialkylamino, C(O)NR^{c}R^{d}, C(O)OR^{a}, NR^{c}C(O)R^{d}, NR^{c}S(O)₂R^{d}, (C₁₋₄ alkyl)sulfonyl, arylsulfonyl, aryl, heteroaryl, cycloalkyl, or heterocycloalkyl.

In some embodiments of the second aspect of the invention, —W″—X″—Y″-Z″ is halo, cyano, C₁₋₄ cyanoalkyl, nitro, C₁₋₄ nitroalkyl, C₁₋₄ alkyl, C₁₋₄ haloalkyl, C₁₋₄ alkoxy, C₁₋₄ haloalkoxy, OH, C₁₋₈ alkoxyalkyl, amino, C₁₋₄ alkylamino, C₂₋₈ dialkylamino, aryl, heteroaryl, cycloalkyl, heterocycloalkyl, arylalkyl, heteroarylalkyl, cycloalkylalkyl, or heterocycloalkylalkyl.

In some embodiments of the second aspect of the invention:

Cy is phenyl optionally substituted by 1, 2, 3, 4 or 5 R^{1a};

L is absent or (CR⁶R⁷)ₘ;

R¹ and R² together with the carbon atom to which they are attached form cyclopropyl;

R³ is H, cyclopropyl, or C₁₋₆ alkyl;

R⁴ is cyclopropyl, (CR^{4a}R^{4b})ₙCy², (CR^{4a}R^{4b})ₙCy³, or —CR^{6a}R^{7a}R^{8a}, wherein said cyclopropyl is optionally substituted by 1, 2 or 3 halo, C₁₋₃ alkyl, C₁₋₃ haloalkyl, phenyl, benzyl, C(O)OR^{10a} or OR^{10a};

R⁶ and R⁷ are each, independently, H, halo, C₁₋₄ alkyl, C₁₋₄ haloalkyl, aryl, cycloalkyl, heteroaryl, heterocycloalkyl, CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}, NR^{c′}R^{d′}, NR^{c′}C(O)R^{d′}, NR^{c′}C(O)OR^{a′}, S(O)R^{b′}, S(O)NR^{c′}R^{d′}, S(O)₂R^{b′}, or S(O)₂NR^{c′}R^{d′};

R^{1a} and R^{1b} are each, independently, halo, CN, NO₂, OH, OR^{a}, SR^{a}, C(O)R^{b}, C(O)NR^{c}R^{d}, C(O)O^{a}, OC(O)R^{b}, OC(O)NR^{c}R^{d}, NR^{c}R^{d}, NR^{c}C(O)R^{d}, NR^{c}C(O)OR^{a}, S(O)R^{b}, S(O)NR^{c}R^{d}, S(O)₂R^{b}, S(O)₂NR^{c}R^{d}, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, aryl, cycloalkyl, heteroaryl or heterocycloalkyl, wherein said C₁₋₆ alkyl, C₂₋₆ alkenyl, C₂₋₆ alkynyl, aryl, cycloalkyl, heteroaryl or heterocycloalkyl is optionally substituted by 1, 2 or 3 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, S(O)R^{b}, S(O)NR^{c}R^{d}, S(O)₂R^{b}, or S(O)₂NR^{c}R^{d};

R^{4a} and R^{4b} are each, independently, H, halo, OH, CN, C₁₋₄alkyl, C₁₋₄ alkoxy, wherein said C₁₋₄ alkyl or C₁₋₄ alkoxy is optionally substituted with one or more halo, CN, NO₂, OH, C₁₋₄ alkoxy, C₁₋₄ haloalkoxy, amino, C₁₋₄ alkylamino or C₂₋₈ dialkylamino;

R^{5a} and R^{5b} are each, independently, H, halo, OH, CN, C₁₋₄alkyl, C₁₋₄ alkoxy, wherein said C₁₋₄ alkyl or C₁₋₄ alkoxy is optionally substituted with one or more halo, CN, NO₂, OH, C₁₋₄ alkoxy, C₁₋₄ haloalkoxy, amino, C₁₋₄ alkylamino or C₂₋₈ dialkylamino;

R^{6a} is H or methyl;

R^{7a} is methyl or CH₂OH;

R^{8a} is C₂₋₆ alkyl or —(CR^{5a}R^{5b})ₚR^{9a}, wherein said C₂₋₆ alkyl is optionally substituted with one or more halo, CN, NO₂, OH, C₁₋₄ alkoxy or C₁₋₄ haloalkoxy;

R^{9a} is halo, CN, NO₂, OH, C₁₋₄ alkoxy, C₁₋₄ haloalkoxy, amino, C₁₋₄ alkylamino, C₂₋₈ dialkylamino, OR^{10b}, SR^{10b}, C(O)R^{10b}, C(O)NR^{10b}R^{11b}, C(O)OR^{10b}, OC(O)R^{10b}, OC(O)NR^{10b}R^{11b}, NR^{10b}R^{11b}, NR^{10b}C(O)R^{11b}, NR^{10b}C(O)OR^{11b}, S(O)R^{10b}, S(O)NR^{10b}R^{11b}, S(O)₂R^{11b}, S(O)₂NR^{10b}R^{11b}, cycloalkyl, aryl, heteroaryl, wherein said cycloalkyl, aryl or heteroaryl is optionally substituted by one or more halo, C₁₋₄ alkyl, C₁₋₄ haloalkyl, CN, NO₂, OH, C₁₋₄ alkoxy, C₁₋₄ haloalkoxy, amino, C₁₋₄ alkylamino or C₂₋₈ dialkylamino;

R^{10a} is H, C₁₋₆ alkyl, C₁₋₆ haloalkyl, C₂₋₆ alkenyl, C2-6 alkynyl, aryl, cycloalkyl, heteroaryl or heterocycloalkyl;

R^{10b} and R^{11b} are each, independently, H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, cycloalkylalkyl, heteroarylalkyl or heterocycloalkylalkyl;

or R^{10b} and R^{11b} together with the N atom to which they are attached form a 4-, 5-, 6- or 7-membered heterocycloalkyl group;

Cy² is:

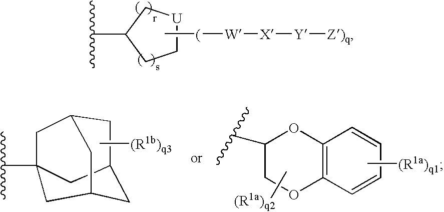

Cy³ is phenyl optionally substituted by 1, 2, 3, 4 or 5 R^{1b};

U is CH₂, NH, or O;

W′—X′—Y′-Z′ is halo, CN, NO₂, OH, C₁₋₄ alkoxy, C₁₋₄ haloalkoxy, amino, C₁₋₄ alkylamino, C₂₋₈ dialkylamino, C₂₋₆ alkyl, C₂₋₆ alkenyl, C₂₋₆ alkynyl, aryl, cycloalkyl, wherein said C₂₋₆ alkyl, C₂₋₆ alkenyl, C₂₋₆ alkynyl, aryl, or cycloalkyl is optionally substituted by 1, 2 or 3 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, S(O)R^{b}, S(O)NR^{c}R^{d}, S(O)₂R^{b}, or S(O)₂NR^{c}R^{d};

W″ is absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, or NR^{e}CONR^{f}, wherein said C₁₋₆ alkylenyl, C₂₋₆ alkenylenyl, C₂₋₆ alkynylenyl are each optionally substituted by 1, 2 or 3 halo, OH, C₁₋₄ alkoxy, C₁₋₄ haloalkoxy, amino, C₁₋₄ alkylamino or C₂₋₈ dialkylamino;

X″ is absent, C₁₋₆ alkylenyl, C₂₋₆ alkenylenyl, C₂₋₆ alkynylenyl, aryl, cycloalkyl, heteroaryl or heterocycloalkyl, wherein said C₁₋₆ alkylenyl, C₂₋₆ alkenylenyl, C₂₋₆ alkynylenyl, cycloalkyl, heteroaryl or heterocycloalkyl is optionally substituted by one or more halo, CN, NO₂, OH, C₁₋₄ alkoxy, C₁₋₄ haloalkoxy, amino, C₁₋₄ alkylamino or C₂₋₈ dialkylamino;

Y″ is absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, or NR^{e}CONR^{f}, wherein said C₁₋₆ alkylenyl, C₂₋₆ alkenylenyl, C₂₋₆ alkynylenyl are each optionally substituted by 1, 2 or 3 halo, OH, C₁₋₄ alkoxy, C₁₋₄ haloalkoxy, amino, C₁₋₄ alkylamino or C₂₋₈ dialkylamino;

Z″ is H, halo, CN, NO₂, OH, C₁₋₄ alkoxy, C₁₋₄ haloalkoxy, amino, C₁₋₄ alkylamino or C₂₋₈ dialkylamino, C₁₋₆ alkyl, C₂₋₆ alkenyl, C₂₋₆ alkynyl, aryl, cycloalkyl, heteroaryl or heterocycloalkyl, wherein said C₁₋₆ alkyl, C₂₋₆ alkenyl, C₂₋₆ alkynyl, aryl, cycloalkyl, heteroaryl or heterocycloalkyl is optionally substituted by 1, 2 or 3 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NRCR^{d}, C(O)OR^{a}, OC(O)R^{b}, OC(O)NR^{c}R^{d}, NR^{c}R^{d}, NR^{c}C(O)R^{d}, NR^{c}C(O)OR^{a}, S(O)R^{b}, S(O)NR^{c}R^{d}, S(O)₂R^{b}, or S(O)₂NR^{c}R^{d};

wherein two —W′—X′—Y′-Z′ together with the atom to which they are both attached optionally form a 3-20 membered cycloalkyl group or 3-20 membered heterocycloalkyl group, each optionally substituted by 1, 2 or 3-W″—X″—Y″-Z″;

or wherein two —W′—X′—Y′-Z′ together with the carbon atom to which they are both attached optionally form a carbonyl;

or wherein two —W′—X′—Y′-Z′ together with two adjacent atoms to which they are attached optionally form a 5- or 6-membered fused aryl optionally substituted by 1, 2 or 3 —W″—X″—Y″-Z″;

wherein —W″—X″—Y″-Z″ is other than H;

R^{a} and R^{a′} are each, independently, H, C₁₋₆ alkyl, C₁₋₆ haloalkyl, C₂₋₆ alkenyl, C₂₋₆ alkynyl, aryl, cycloalkyl, heteroaryl or heterocycloalkyl;

R^{b} and R^{b′} are each, independently, H, C₁₋₆ alkyl, C₁₋₆ haloalkyl, C₂₋₆ alkenyl, C₂₋₆ alkynyl, aryl, cycloalkyl, heteroaryl or heterocycloalkyl;

R^{c} and R^{d} are each, independently, H, C₁₋₆ alkyl, C₁₋₆ haloalkyl, C₂₋₆ alkenyl, C₂₋₆ alkynyl, aryl, cycloalkyl, arylalkyl, or cycloalkylalkyl;

or R^{c} and R^{d} together with the N atom to which they are attached form a 4-, 5-, 6- or 7-membered heterocycloalkyl group;

R^{c′} and R^{d′} are each, independently, H, C₁₋₆ alkyl, C₁₋₆ haloalkyl, C₂₋₆ alkenyl, C₂₋₆ alkynyl, aryl, cycloalkyl, arylalkyl, or cycloalkylalkyl;

or R^{c′} and R^{d} together with the N atom to which they are attached form a 4-, 5-, 6- or 7-membered heterocycloalkyl group;

R^{e} and R^{f} are each, independently, H, C₁₋₆ alkyl, C₁₋₆ haloalkyl, C₂₋₆ alkenyl, C₂₋₆ alkynyl, aryl, cycloalkyl, arylalkyl, or cycloalkylalkyl;

or R^{e} and R^{f} together with the N atom to which they are attached form a 4-, 5-, 6- or 7-membered heterocycloalkyl group;

m is 1, 2, 3 or 4;

n is 0, 1, 2, or 3;

t is 2 or 3;

s is 1 or 2;

p is 1, 2, 3, 4 or 5;

q1 is 0, 1, 2, 3 or 4;

q2 is 0, 1, 2 or 3;

q3 is 1, 2 or 3;

q is 0, 1, 2, 3, 4 or 5; and

r is 1 or 2.

In further embodiments of the second aspect of the invention, R³ is H or cyclopropyl.

In further embodiments of the second aspect of the invention, R³ is H.

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

The term “n-membered” where n is an integer typically describes the number of ring-forming atoms in a moiety where the number of ring-forming atoms is n. For example, piperidinyl is an example of a 6-membered heterocycloalkyl ring and 1,2,3,4-tetrahydro-naphthalene is an example of a 10-membered cycloalkyl group.

For compounds of the invention in which a variable appears more than once, each variable can be a different moiety selected from the Markush group defining the variable. For example, where a structure is described having two R groups that are simultaneously present on the same compound; the two R groups can represent different moieties selected from the Markush group defined for R. In another example, when an optionally multiple substituent is designated in the form: 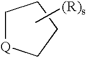
then it is understood that substituent R can occur s number of times on the ring, and R can be a different moiety at each occurrence. Further, in the above example, should the variable Q be defined to include hydrogens, such as when Q is said to be CH₂, NH, etc., any floating substituent such as R in the above example, can replace a hydrogen of the Q variable as well as a hydrogen in any other non-variable component of the ring.

It is further intended that the compounds of the invention are stable. As used herein “stable” refers to a compound that is sufficiently robust to survive isolation to a useful degree of purity from a reaction mixture, and preferably capable of formulation into an efficacious therapeutic agent.

As used herein, the term “alkyl” is meant to refer to a saturated hydrocarbon group which is straight-chained or branched. Example alkyl groups include methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like. An alkyl group can contain from 1 to about 20, from 2 to about 20, from 1 to about 10, from 1 to about 8, from 1 to about 6, from I to about 4, or from 1 to about 3 carbon atoms. The term “alkylenyl” refers to a divalent alkyl linking group.

As used herein, “alkenyl” refers to an alkyl group having one or more double carbon-carbon bonds. Example alkenyl groups include ethenyl, propenyl, cyclohexenyl, and the like. The term “alkenylenyl” refers to a divalent linking alkenyl group.

As used herein, “alkynyl” refers to an alkyl group having one or more triple carbon-carbon bonds. Example alkynyl groups include ethynyl, propynyl, and the like. The term “alkynylenyl” refers to a divalent linking alkynyl group.

As used herein, “haloalkyl” refers to an alkyl group having one or more halogen substituents. Example haloalkyl groups include CF₃, C₂F₅, CHF₂, CCl₃, CHCl₂, C₂Cl₅, and the like.

As used herein, “aryl” refers to monocyclic or polycyclic (e.g., having 2, 3 or 4 fused rings) aromatic hydrocarbons such as, for example, phenyl, naphthyl, anthracenyl, phenanthrenyl, indanyl, indenyl, and the like. In some embodiments, aryl groups have from 6 to about 20 carbon atoms.

As used herein, “cycloalkyl” refers to non-aromatic cyclic hydrocarbons including cyclized alkyl, alkenyl, and alkynyl groups. Cycloalkyl groups can include mono- or polycyclic (e.g., having 2, 3 or 4 fused rings) ring systems as well as spiro ring systems. Ring-forming carbon atoms of a cycloalkyl group can be optionally substituted by oxo or sulfido. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like. Also included in the definition of cycloalkyl are moieties that have one or more aromatic rings fused (i.e., having a bond in common with) to the cycloalkyl ring, for example, benzo or thienyl derivatives of pentane, pentene, hexane, and the like.

As used herein, “heteroaryl” groups refer to an aromatic heterocycle having at least one heteroatom ring member such as sulfur, oxygen, or nitrogen. Heteroaryl groups include monocyclic and polycyclic (e.g., having 2, 3 or 4 fused rings) systems. Examples of heteroaryl groups include without limitation, pyridyl, pyrimidinyl, pyrazinyl, pyridazinyl, triazinyl, furyl, quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, and the like. In some embodiments, the heteroaryl group has from 1 to about 20 carbon atoms, and in further embodiments from about 3 to about 20 carbon atoms. In some embodiments, the heteroaryl group contains 3 to about 14, 3 to about 7, or 5 to 6 ring-forming atoms. In some embodiments, the heteroaryl group has 1 to about 4, 1 to about 3, or 1 to 2 heteroatoms.

As used herein, “heterocycloalkyl” refers to non-aromatic heterocycles including cyclized alkyl, alkenyl, and alkynyl groups where one or more of the ring-forming carbon atoms is replaced by a heteroatom such as an O, N, or S atom. Heterocycloalkyl groups can be mono- or polycyclic (e.g., having 2, 3, 4 or more fused rings or having a 2-ring, 3-ring, 4-ring spiro system (e.g., having 8 to 20 ring-forming atoms)). Example “heterocycloalkyl” groups include morpholino, thiomorpholino, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, isothiazolidinyl, pyrazolidinyl, oxazolidinyl, thiazolidinyl, imidazolidinyl, and the like. Ring-forming carbon atoms and heteroatoms of a heterocycloalkyl group can be optionally substituted by oxo or sulfido. Also included in the definition of heterocycloalkyl are moieties that have one or more aromatic rings fused (i.e., having a bond in common with) to the nonaromatic heterocyclic ring, for example phthalimidyl, naphthalimidyl, and benzo derivatives of heterocycles such as indolene and isoindolene groups. In some embodiments, the heterocycloalkyl group has from I to about 20 carbon atoms, and in further embodiments from about 3 to about 20 carbon atoms. In some embodiments, the heterocycloalkyl group contains 3 to about 14, 3 to about 7, or 5 to 6 ring-forming atoms. In some embodiments, the heterocycloalkyl group has 1 to about 4, 1 to about 3, or 1 to 2 heteroatoms. In some embodiments, the heterocycloalkyl group contains 0 to 3 double bonds. In some embodiments, the heterocycloalkyl group contains 0 to 2 triple bonds.

As used herein, “halo” or “halogen” includes fluoro, chloro, bromo, and iodo.

As used herein, “alkoxy” refers to an —O-alkyl group. Example alkoxy groups include methoxy, ethoxy, propoxy (e.g., n-propoxy and isopropoxy), t-butoxy, and the like.

As used here, “haloalkoxy” refers to an —O-haloalkyl group. An example haloalkoxy group is OCF₃.

As used herein, “arylalkyl” refers to alkyl substituted by aryl and “cycloalkylalkyl” refers to alkyl substituted by cycloalkyl. An example arylalkyl group is benzyl.

As used herein, “amino” refers to NH₂.

As used herein, “alkylamino” refers to an amino group substituted by an alkyl group.

As used herein, “dialkylamino” refers to an amino group substituted by two alkyl groups.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms.

Resolution of racemic mixtures of compounds can be carried out by any of numerous methods known in the art. An example method includes fractional recrystallizaion using a “chiral resolving acid” which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as P-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds of the invention also include tautomeric forms, such as keto-enol tautomers.

Compounds of the invention can also include all isotopes of atoms occurring in the intermediates or final compounds. Isotopes include those atoms having the same atomic number but different mass numbers. For example, isotopes of hydrogen include tritium and deuterium.

The phrase “pharmaceutically acceptable” is employed herein to refer to those compounds, materials, compositions, and/or dosage forms which are, within the scope of sound medical judgement, suitable for use in contact with the tissues of human beings and animals without excessive toxicity, irritation, allergic response, or other problem or complication, commensurate with a reasonable benefit/risk ratio.

The present invention also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts or the quaternary ammonium salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

The present invention also includes prodrugs of the compounds described herein. As used herein, “prodrugs” refer to any covalently bonded carriers which release the active parent drug when administered to a mammalian subject. Prodrugs can be prepared by modifying functional groups present in the compounds in such a way that the modifications are cleaved, either in routine manipulation or in vivo, to the parent compounds. Prodrugs include compounds wherein hydroxyl, amino, sulfhydryl, or carboxyl groups are bonded to any group that, when administered to a mammalian subject, cleaves to form a free hydroxyl, amino, sulfhydryl, or carboxyl group respectively. Examples of prodrugs include, but are not limited to, acetate, formate and benzoate derivatives of alcohol and amine functional groups in the compounds of the invention. Preparation and use of prodrugs is discussed in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference in their entirety.

Synthesis

The novel compounds of the present invention can be prepared in a variety of ways known to one skilled in the art of organic synthesis. The compounds of the present invention can be synthesized using the methods as hereinafter described below, together with synthetic methods known in the art of synthetic organic chemistry or variations thereon as appreciated by those skilled in the art.

The compounds of this invention can be prepared from readily available starting materials using the following general methods and procedures. It will be appreciated that where typical or preferred process conditions (i.e., reaction temperatures, times, mole ratios of reactants, solvents, pressures, etc.) are given; other process conditions can also be used unless otherwise stated. Optimum reaction conditions may vary with the particular reactants or solvent used, but such conditions can be determined by one skilled in the art by routine optimization procedures.

The processes described herein can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatograpy (HPLC) or thin layer chromatography.

Preparation of compounds can involve the protection and deprotection of various chemical groups. The need for protection and deprotection, and the selection of appropriate protecting groups can be readily determined by one skilled in the art. The chemistry of protecting groups can be found, for example, in Greene, et al., Protective Groups in Organic Synthesis, 2d. Ed., Wiley & Sons, 1991, which is incorporated herein by reference in its entirety.

The reactions of the processes described herein can be carried out in suitable solvents which can be readily selected by one of skill in the art of organic synthesis. Suitable solvents can be substantially nonreactive with the starting materials (reactants), the intermediates, or products at the temperatures at which the reactions are carried out, i.e., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected.

The compounds of the invention can be prepared, for example, using the reaction pathways and techniques as described below.

A series of cyclopropanecarboxamides and cyclobutanecarboxamides of formula 2 wherein Cy is aryl, heteroaryl, cycloalkyl, heterocycloalkyl or the derivatives thereof can be prepared by the method outlined in Scheme 1. Cyclopropane- or cyclobutane-carboxylic acid 1 can be coupled to an appropriate amine NHR³R⁴ (primary or secondary) using a coupling reagent such as BOP to provide the desired product 2.

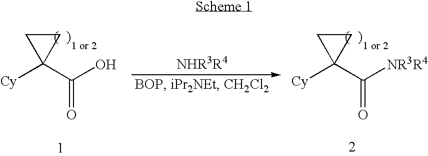

A series of cyclopropanecarboxylic acids and cyclobutanecarboxylic acids of formula 3 can be prepared by the method outlined in Scheme 2. Mono-alkylation of alpha-substituted methyl ester 4 with either ethylene bromide or 1,3-dibromopropane provides mono-alkylated product 5, which upon treatment with a suitable base such as sodium hydride or LDA in a suitable solvent such as DMSO, DMF or THF yields cyclopropanecarboxylates and cyclobutanecarboxylates 6, respectively. Finally basic hydrolysis of 6 gives the corresponding carboxylic acids 3.

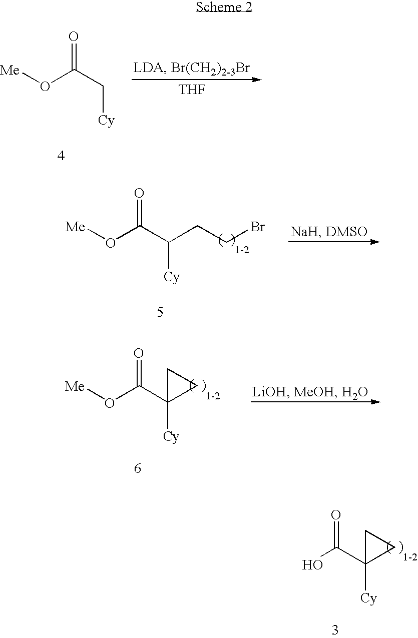

A series of cyclobutanecarboxylic acids of formula 7 can be prepared by the method outlined in Scheme 3. Alpha-substituted acetonitrile 8 can be treated with potassium hydroxide and 1,3-dibromopropane to provide substituted cyclobutanecarbonitrile 8a, followed by hydrolysis to afford the desired cyclobutanecarboxylic acid 7.

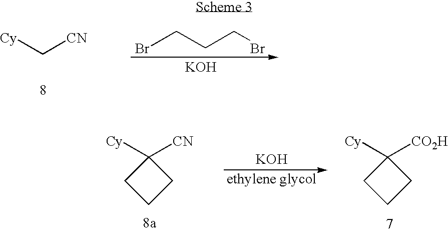

Primary amines of formula 10, wherein R^{x} can be a variety of substituents such as alkyl, cycloalkyl or aryl, can be prepared from the appropriate cyclic ketone 9 under a variety of protocols, one of which is shown in Scheme 4. The ketone of compound 9 undergoes reductive amination with ammonium formamide to afford the amine compound 10.

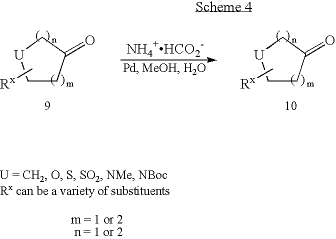

Alternatively, primary amines 10 can be prepared from the appropriate alcohols 11 via mesylation, followed by conversion of the mesylates 12 to the corresponding azides 13, which upon reduction yield the desired primary amines 10, as shown in Scheme 5.

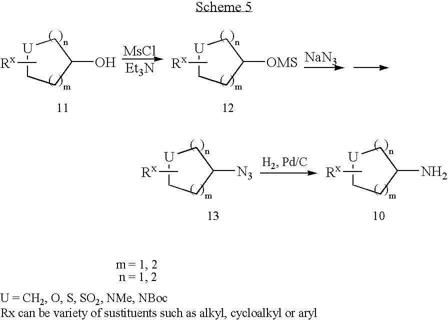

Cyclopropane or cyclobutanecarboxamides of formula 14 can be prepared as shown in Scheme 6 (U, R^{x}, m and n are as defined in Schemes 4 and 5) using BOP or any other suitable coupling reagent.

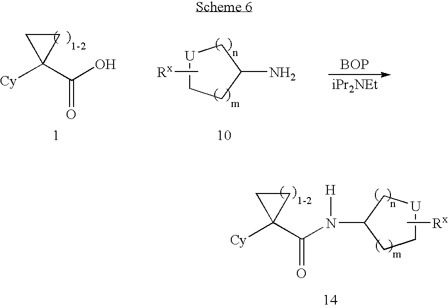

Cyclopropane- or cyclobutane-carboxamides of formula 18 can be prepared according to the method outlined in Scheme 7 (U, R^{x}, m and n are as defined in Schemes 4 and 5). Standard coupling of carboxylic acids 1 with an appropriate primary amine 15 provides carboxamides 16. Cleavage of the N-Boc group with TFA gives compounds 17, which can be converted by routine methods to carboxamides 18.

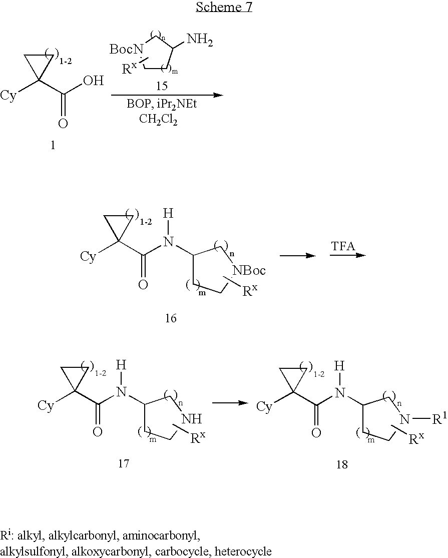

Secondary amines of formula 19 can be prepared from the reaction of an appropriate cyclic amine 10 with a suitable aldehyde R′CHO (wherein R′ can be H, alkyl, cylcoalkyl, heterocycloalyl or the like) and a reducing reagent such as Na CNBH₃ as shown in Scheme 8 (U, R^{x}, m and n are as defined in Schemes 4 and 5).

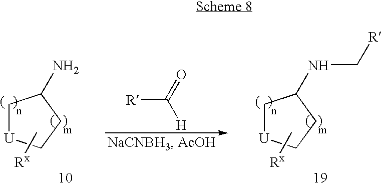

Carboxamides of formula 20 can be prepared in the standard fashion by using a coupling reagent and a base as shown in Scheme 9 (U, R^{x}, m and n are as defined in Schemes 4 and 5; R′ is as defined in Scheme 8).

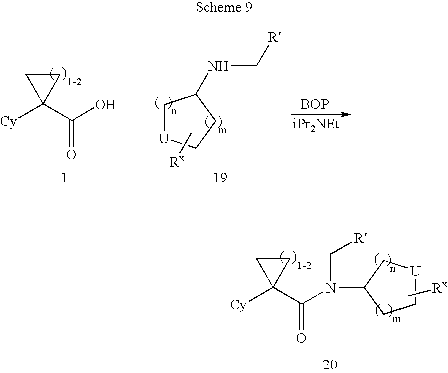

Alternatively, cyclopropane- and cyclobutane-carboxamides of formula 22 can be prepared following the sequence outlined in Scheme 10. Standard coupling of carboxylic acids 1 with an appropriate primary amine R³NH₂ wherein R³ can be alkyl, cycloalkyl, heterocycloalkylalkyl or cycloalkylalkyl, provides carboxamides 21 which upon alkylation with a suitable bromide or iodide R⁴X can be converted to the desired compounds 22, wherein R⁴ can be alkyl, cycloalkyl or heterocycloalkyl, each optionally substituted by a variety of suitable substituents.

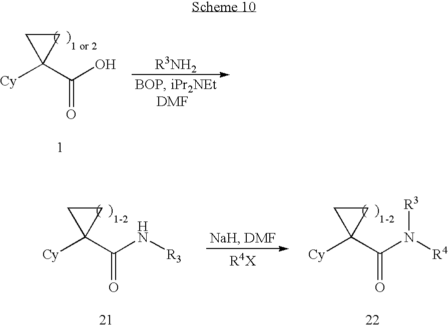

Primary amines of formula 25 and secondary amines of formula 26 can be prepared according to the method outlined in Scheme 11 (wherein Ar can be an aromatic moiety, arylalkyl or the like, R is alkyl, and R′ is alkyl, cycloalkyl, aryl, heterocycloalkyl, heteroaryl, etc.). A suitable bromide such as 23 can be converted to the corresponding azide 24 first, and then to the desired primary amine 25 via hydrogenation. Finally reductive amination with an appropriate aldehyde R′CHO (wherein R′ can be H, alkyl, cylcoalkyl, heterocycloalyl or the like) yields secondary amines of formula 26.

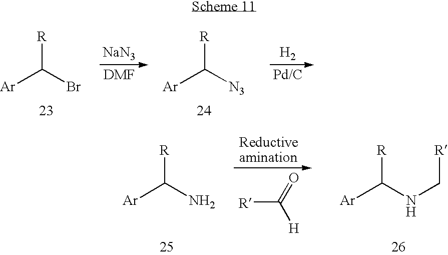

Amines of formula 32 can be prepared according to the method outlined in Scheme 12 (Rⁱⁱⁱ and R^{iv} are each, independently, e.g., H, alkyl, halo, haloalkyl, aryl, heteroaryl, cycloalkyl, heterocycloalkyl, etc.). An appropriate substituted o-hydroxycetophenones 27, available by Fries rearrangement, can react with epichlorohydrin and base to give the corresponding ethers 28. Subjecting 28 to Baeyer-Villiger oxidation provides the acetoxy intermediates 29, which can be saponified and cyclized in one step to provide alcohols 30. Oxidation of the alcohols 30 gives the corresponding aldehydes 31 with TPAP and NMO. The aldehydes 31 can undergo reductive amination with a desired primary amine to afford the desired compounds 32.

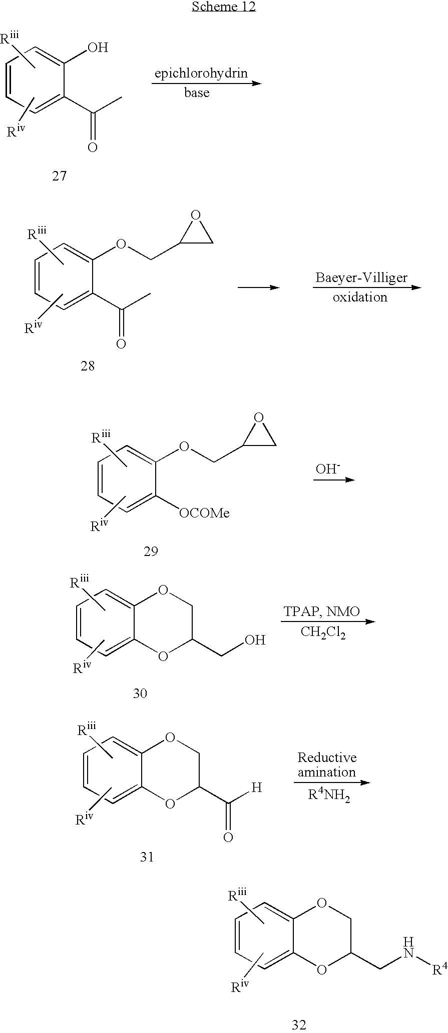

Primary amines 36 and secondary amines 37 can be prepared according to the method outlined in Scheme 13 (Rⁱⁱⁱ and R^{iv} are each, independently, e.g., H, alkyl, halo, haloalkyl, aryl, heteroaryl, cycloalkyl, heterocycloalkyl, etc; R^{v} is, e.g., alkyl, halo, haloalkyl, aryl, heteroaryl, cycloalkyl, heterocycloalkyl, etc. R′ can be H, alkyl, cylcoalkyl, heterocycloalyl, etc.). Reaction of a substituted indole 33 with an Fmoc-protected amino acid chloride 34, followed by cleavage of the Fmoc group with piperidine in DMF provides a ketone compound 35. Reduction of the carbonyl group of 35 with NaBH4 gives a primary amine compound 36 which upon treatment with an appropriate aldehyde R′CHO under reductive amination conditions provides a secondary amine 37.

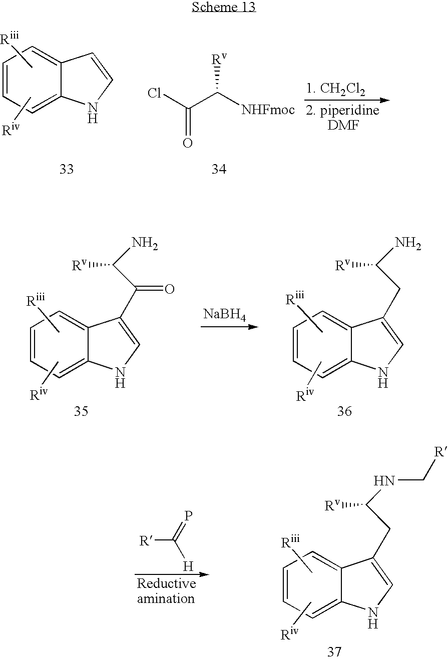

A series of compounds 42 can be prepared by the method outlined in Scheme 14 (R is, e.g., alkyl, cycloalkyl, aryl, heteroarl, etc.; X is halo or other leaving group; R is alkyl, cycloalkyl, etc.). Compound 38 can be treated with a dibromoalkane BrCH₂(CH₂)ₘBr wherein n is 1 to 6, such as 1,2-dibromoethane, to give the desired cycloalkyl product 39. Both benzyl (Bn) groups of 39 can be removed by hydrogenation to give deprotected compound 40. Treatment with amines NHR³R⁴ can provide amides of formula 41. The amines NHR³R⁴ can be selected from a variey primary or secondary amines. The free hydroxyl group of 41 can be converted to a variety of ether analogs 42 by routine methods.

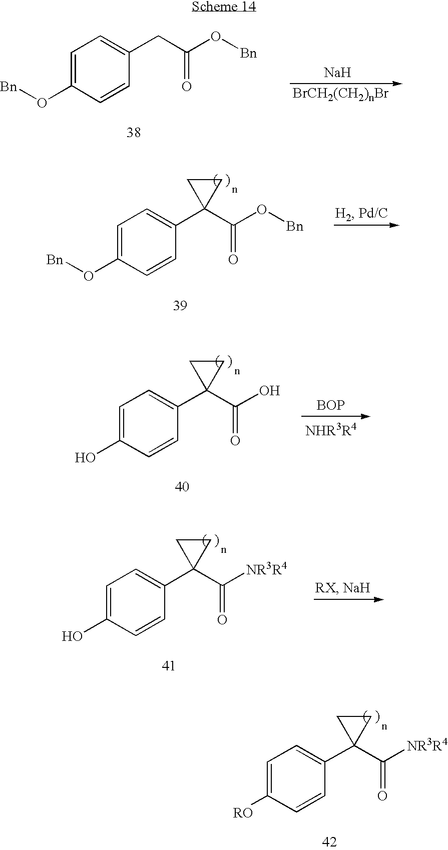

A series of compounds 44 can be prepared by the method outlined in Scheme 15 wherein n is 1-6 and Ar is aryl, heteroaryl, or substituted thereof. Phenols 41 can be converted to the corresponding triflates 43 which then can undergo Pd catalyzed Suzuki coupling to provide compounds 44.

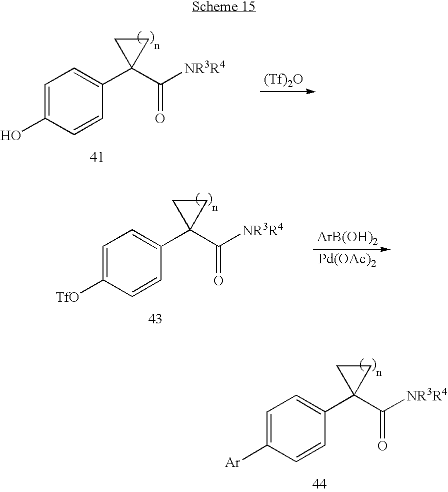

A series of compounds 45 can be prepared by the method outlined in Scheme 16 (Ar can be, for example, aryl or heteroaryl or derivatives thereof, n is 1-6). The free phenol group of 41 can be coupled with ArB(OH)₂ directly to provide the aryl- or heteroaryl-ether product 45.

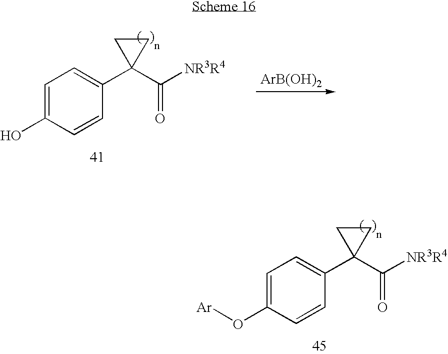

A series of heterocycloalkyl- or heterocylcoalkylalkyl-ether compounds 46 and 47 can be prepared by the method outlined in Scheme 17 (n is 1-6; U is, e.g., O, N-alkyl, etc.). The free phenol of 41 can be treated with a variety of heterocycloalkyl triflates, heterocycloalkylalkyl halides or heterocycloalkylalkyl triflates to provide heterocycloalkyl- or heterocylcoalkylalkyl-ether compounds 46 and 47.

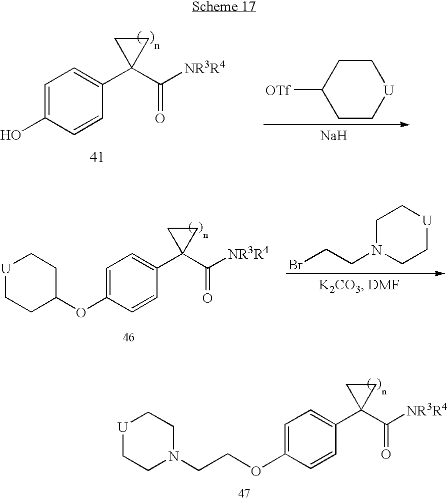

A series of cylcoalkanecarboxamides such as cyclopropanecarboxamides and cyclobutanecarboxamides of formula 48 can be prepared by the method outlined in Scheme 18. Carboxylic acids of formula 48a can be coupled to an amine using a coupling reagent such as BOP to provide the desired compounds 48 wherein L can be S, (CH₂)ₘS, (CH₂)ₘO, (CH₂)ₘ, etc.

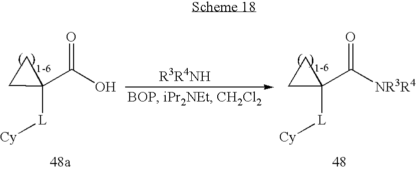

A series of cyclopropane- and cyclobutane-carboxylic acids of formula 52, wherein L can be S, can be prepared according to the method outlined in Scheme 19. Reaction of the appropriate thiol 49 with methyl bromoacetate in the presence of a base such as potassium or sodium carbonate, triethylamine or sodium hydride in a solvent such as tetrahydrofuran, acetonitrile or dichloromethane provides thioethers 50. Treatment of 50 with a dihaloalkane such as 1,2-dibromoethane or 1,3-dibromopropane in the presence of sodium hydride, ether and DMSO provides methyl esters 51, which upon basic hydrolysis yield the desired carboxylic acids 52.

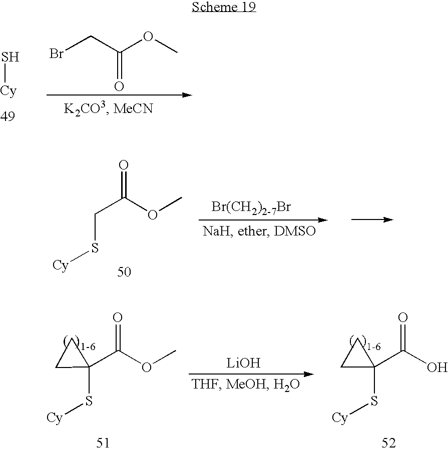

Alternatively, starting with an appropriate cyclo-thioketone 53 and following Scheme 20, a series of carboxylic acids of formula 56 wherein the ring is aromatic or non-aromatic can be prepared.

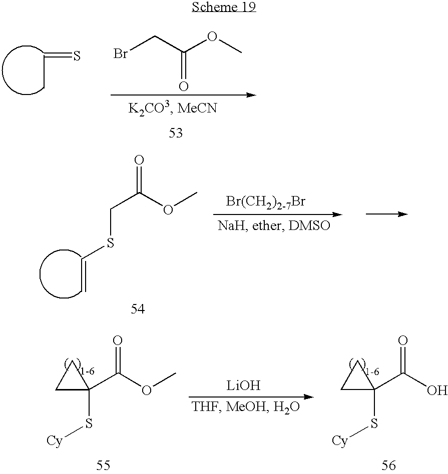

A series of carboxylic acids of formula 62 can be prepared by the method outlined in Scheme 21. S-alkylation of mercaptoacetic acid 57 with a suitable chloride or bromide CyCH₂X provides carboxylic acids 58, which can be converted to the corresponding methyl esters 59. Monoalkylation of 59 with a dihaloalkane such as 1,2-dibromoethane or 1,3-dibromopropane in the presence of LDA yields methyl esters 60, which upon treatment with either NaH in DMSO or DMF or LDA in THF provide the corresponding esters 61. Finally, basic hydrolysis yields the desired carboxylic acids 62.

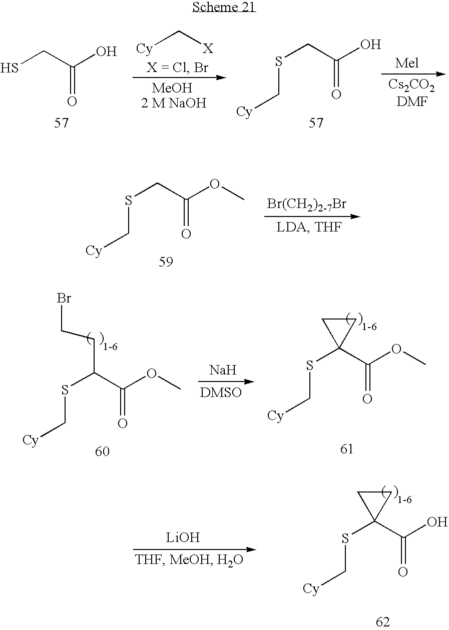

Alternatively, a series of carboxylic acids of formula 66, wherein m is 1 or 2 and Cy is a cyclic moiety such as aryl, can be prepared according to Scheme 22. Reaction of an appropriate thiol 63 with chloroacetonitrile in the presence of a base such as sodium ethoxide under refluxing conditions provides nitriles 64. Treatment of 64 with a dihaloalkane such as 1,2-dibromoethane or 1,3-dibromopropane under any of the conditions shown below yields the corresponding cyclopropane or cyclobutanenitriles 65, which upon basic hydrolysis provide the desired carboxylic acids 66.

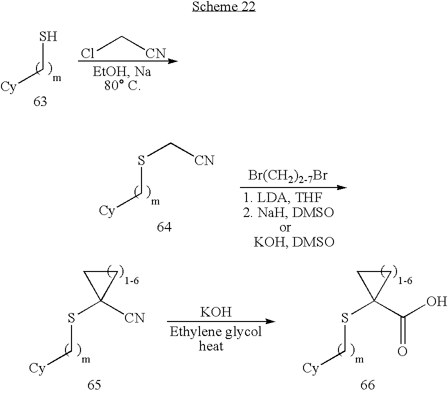

Alternatively, (such as when Cy is heteroaryl) carboxylic acids 71 can be prepared by the reaction of an appropriate alcohol with thioglycolic acid 57 in the presence of a Lewis acid such as zinc trifluoromethanesulfonate, under refluxing conditions. Then acids 67 can be processed to the desired carboxylic acids 71 in the standard fashion as shown in Scheme 23.

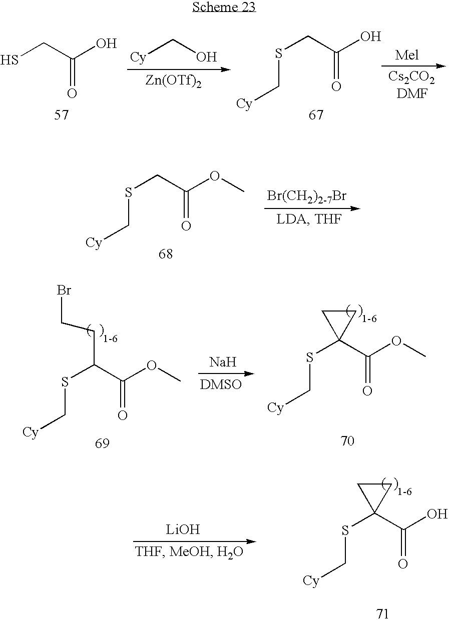

As shown in scheme 24, thioether 50 can be oxidized to the corresponding sulfone 72 with 3-chloroperoxybenzoic acid. Following scheme 24, as previously described, a series of carboxylic acids of formula 74 can be prepared. The same sequence (conversion of the thioether to a sulfone) can be employed in all the schemes described earlier.

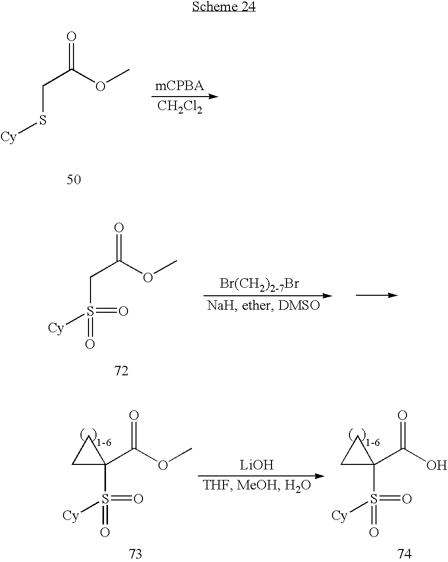

A series of carboxylic acids of formula 78, can be prepared according to the method outlined in Scheme 25. Commercially available hydroxyacid 75 can be converted to the corresponding methyl ester 76, which can react with the appropriate bromide or chloride CyCH₂X in the presence of a suitable base such as NaH or K₂CO₃ and in a suitable solvent such as DMF to yield methyl esters 77. Basic hydrolysis of 77 provides the desired carboxylic acids 78 wherein Cy is a cyclic moiety such as aryl.

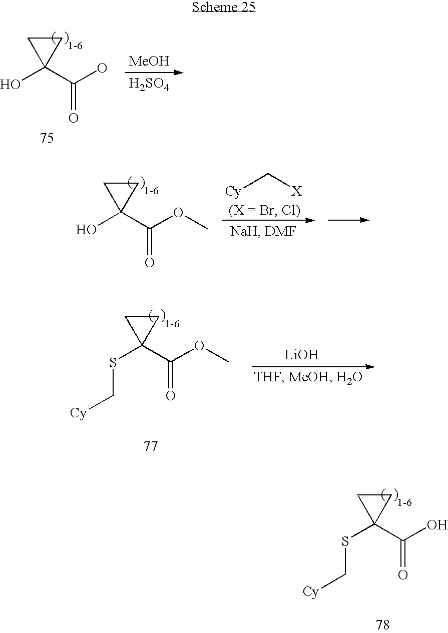

A series of carboxylic acids of formula 82 (R′ and R″ can each be halogen, alkyl, haloalkyl and the like) can be prepared according to Scheme 26. Reaction of a suitable phenol 79 with 2-chloromethyl acetate in the presence of KI and K₂CO₃ in refluxing acetone provides methyl esters 80, which can be converted to the desired carboxylic acids 82 in the standard fashion, as depicted in Scheme 26.

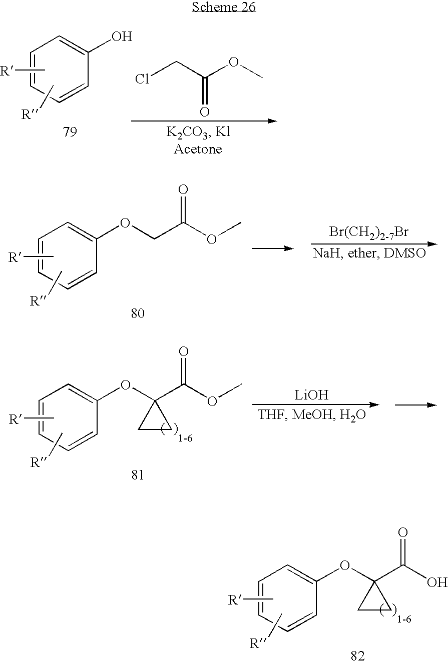

A series of carboxylic acids of formula 87 can be prepared according to Scheme 27. O-alkylation of methyl ester 83 with the appropriate bromide or chloride CyCH₂X provides compounds 84 which can be processed to the desired carboxylic acids 87 wherein Cy is a cyclic moiety such as aryl in the standard fashion, as shown below.

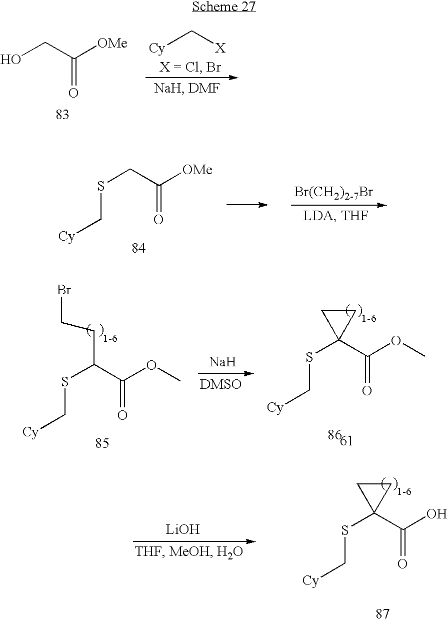

A series of carboxylic acids of formula 90 (wherein m can be 1, 2, 3 or 4, and R⁶ and R⁷ can be H or a variety of suitable substituents such as alkyl, aryl, halo, etc.) can be prepared by the method outlined in Scheme 28. The methyl ester 88 can be alkylated with a suitable a dihaloalkane such as 1,2-dibromoethane or 1,3-dibromopropane to provide 89, which upon basic hydrolysis yields the desired carboxylic acid 90 wherein Cy is a cyclic moiety such as aryl. 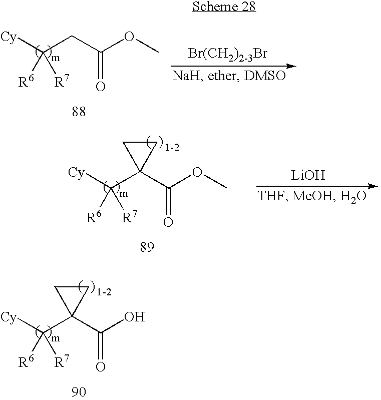
Methods

Compounds of the invention can modulate activity of 11βHSD1 and/or MR. The term “modulate” is meant to refer to an ability to increase or decrease activity of an enzyme or receptor. Accordingly, compounds of the invention can be used in methods of modulating 11βHSD1 and/or MR by contacting the enzyme or receptor with any one or more of the compounds or compositions described herein. In some embodiments, compounds of the present invention can act as inhibitors of 11βHSD1 and/or MR. In further embodiments, the compounds of the invention can be used to modulate activity of 11βHSD1 and/or MR in an individual in need of modulation of the enzyme or receptor by administering a modulating amount of a compound of the invention.

The present invention further provides methods of inhibiting the conversion of cortisone to cortisol in a cell, or inhibiting the production of cortisol in a cell, where conversion to or production of cortisol is mediated, at least in part, by 11βHSD1 activity. Methods of measuring conversion rates of cortisone to cortisol and vice versa, as well as methods for measuring levels of cortisone and cortisol in cells, are routine in the art.

The present invention further provides methods of increasing insulin sensitivity of a cell by contacting the cell with a compound of the invention. Methods of measuring insulin sensitivity are routine in the art.

The present invention further provides methods of treating disease associated with activity or expression, including abnormal activity and overexpression, of 11βHSD1 and/or MR in an individual (e.g., patient) by administering to the individual in need of such treatment a therapeutically effective amount or dose of a compound of the present invention or a pharmaceutical composition thereof. Example diseases can include any disease, disorder or condition that is directly or indirectly linked to expression or activity of the enzyme or receptor. An 11βHSD1-associated disease can also include any disease, disorder or condition that can be prevented, ameliorated, or cured by modulating enzyme activity.

Examples of 11βHSD1-associated diseases include obesity, diabetes, glucose intolerance, insulin resistance, hyperglycemia, hypertension, hyperlipidemia, cognitive impairment, dementia, glaucoma, cardiovascular disorders, osteoporosis, and inflammation. Further examples of 11βHSD1-associated diseases include metabolic syndrome, type 2 diabetes, androgen excess (hirsutism, menstrual irregularity, hyperandrogenism) and polycystic ovary syndrome (PCOS).

The present invention further provides methods of modulating MR activity by contacting the MR with a compound of the invention, pharmaceutically acceptable salt, prodrug, or composition thereof. In some embodiments, the modulation can be inhibition. In further embodiments, methods of inhibiting aldosterone binding to the MR (optionally in a cell) are provided. Methods of measuring MR activity and inhibition of aldosterone binding are routine in the art.

The present invention further provides methods of treating a disease associated with activity or expression of the MR. Examples of diseases associated with activity or expression of the MR include, but are not limited to hypertension, as well as cardiovascular, renal, and inflammatory pathologies such as heart failure, atherosclerosis, arteriosclerosis, coronary artery disease, thrombosis, angina, peripheral vascular disease, vascular wall damage, stroke, dyslipidemia, hyperlipoproteinaemia, diabetic dyslipidemia, mixed dyslipidemia, hypercholesterolemia, hypertriglyceridemia, and those associated with type 1 diabetes, type 2 diabetes, obesity metabolic syndrome, insulin resistance and general aldosterone-related target organ damage.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal. In some embodiments, the cell is an adipocyte, a pancreatic cell, a hepatocyte, neuron, or cell comprising the eye.

As used herein, the term “contacting” refers to the bringing together of indicated moieties in an in vitro system or an in vivo system. For example, “contacting” the 11βHSD1 enzyme with a compound of the invention includes the administration of a compound of the present invention to an individual or patient, such as a human, having 11βHSD1, as well as, for example, introducing a compound of the invention into a sample containing a cellular or purified preparation containing the 11βHSD1 enzyme.

As used herein, the term “individual” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent that elicits the biological or medicinal response that is being sought in a tissue, system, animal, individual or human by a researcher, veterinarian, medical doctor or other clinician, which includes one or more of the following:

(1) preventing the disease; for example, preventing a disease, condition or disorder in an individual who may be predisposed to the disease, condition or disorder but does not yet experience or display the pathology or symptomatology of the disease (non-limiting examples are preventing metabolic syndrome, hypertension, obesity, insulin resistance, hyperglycemia, hyperlipidemia, type 2 diabetes, androgen excess (hirsutism, menstrual irregularity, hyperandrogenism) and polycystic ovary syndrome (PCOS);

(2) inhibiting the disease; for example, inhibiting a disease, condition or disorder in an individual who is experiencing or displaying the pathology or symptomatology of the disease, condition or disorder (i.e., arresting further development of the pathology and/or symptomatology) such as inhibiting the development of metabolic syndrome, hypertension, obesity, insulin resistance, hyperglycemia, hyperlipidemia, type 2 diabetes, androgen excess (hirsutism, menstrual irregularity, hyperandrogenism) or polycystic ovary syndrome (PCOS), stabilizing viral load in the case of a viral infection; and

(3) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of metabolic syndrome, hypertension, obesity, insulin resistance, hyperglycemia, hyperlipidemia, type 2 diabetes, androgen excess (hirsutism, menstrual irregularity, hyperandrogenism) and polycystic ovary syndrome (PCOS), or lowering viral load in the case of a viral infection.

Pharmaceutical Formulations and Dosage Forms

When employed as pharmaceuticals, the compounds of Formula I can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, oral or parenteral. Methods for ocular delivery can include topical administration (eye drops), subconjunctival, periocular or intravitreal injection or introduction by balloon catheter or ophthalmic inserts surgically placed in the conjunctival sac. Parenteral administration includes intravenous, intraarterial, subcutaneous, intraperitoneal or intramuscular injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This invention also includes pharmaceutical compositions which contain, as the active ingredient, one or more of the compounds of the invention above in combination with one or more pharmaceutically acceptable carriers. In making the compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

The compositions can be formulated in a unit dosage form, each dosage containing from about 5 to about 100 mg, more usually about 10 to about 30 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, 0.1 to about 500 mg of the active ingredient of the present invention.

The tablets or pills of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions in can be nebulized by use of inert gases. Nebulized solutions may be breathed directly from the nebulizing device or the nebulizing device can be attached to a face masks tent, or intermittent positive pressure breathing machine. Solution, suspension, or powder compositions can be administered orally or nasally from devices which deliver the formulation in an appropriate manner.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of the compounds of the present invention can vary according to, for example, the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. For example, the compounds of the invention can be provided in an aqueous physiological buffer solution containing about 0.1 to about 10% w/v of the compound for parenteral adminstration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compounds of the invention can also be formulated in combination with one or more additional active ingredients which can include any pharmaceutical agent such as anti-viral agents, antibodies, immune suppressants, anti-inflammatory agents and the like.

Labeled Compounds and Assay Methods

Another aspect of the present invention relates to radio-labeled compounds of the invention that would be useful not only in radio-imaging but also in assays, both in vitro and in vivo, for localizing and quantitating the enzyme in tissue samples, including human, and for identifying ligands by inhibition binding of a radio-labeled compound. Accordingly, the present invention includes enzyme assays that contain such radio-labeled compounds.

The present invention further includes isotopically-labeled compounds of the invention. An “isotopically” or “radio-labeled” compound is a compound of the invention where one or more atoms are replaced or substituted by an atom having an atomic mass or mass number different from the atomic mass or mass number typically found in nature (i.e., naturally occurring). Suitable radionuclides that may be incorporated in compounds of the present invention include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br ⁷⁵Br, ⁷⁶Br, ⁷⁷Br, ¹²³I, ¹²⁴I, ¹²⁵I and ¹³¹I. The radionuclide that is incorporated in the instant radio-labeled compounds will depend on the specific application of that radio-labeled compound. For example, for in vitro receptor labeling and competition assays, compounds that incorporate ³H, ¹⁴C, ⁸²Br, 125I, ¹³¹I, ³⁵S or will generally be most useful. For radio-imaging applications ¹¹C, ¹⁵F, ¹²⁵I, ¹²³I, ¹²⁴I, ¹³¹I, ⁷⁵Br, ⁷⁶Br or ⁷⁷Br will generally be most useful.

It is understood that a “radio-labeled” or “labeled compound” is a compound that has incorporated at least one radionuclide. In some embodiments the radionuclide is selected from the group consisting of ³H, ¹⁴C, 125I , ³⁵S and ⁸²Br.

Synthetic methods for incorporating radio-isotopes into organic compounds are applicable to compounds of the invention and are well known in the art.

A radio-labeled compound of the invention can be used in a screening assay to identify/evaluate compounds. In general terms, a newly synthesized or identified compound (i.e., test compound) can be evaluated for its ability to reduce binding of the radio-labeled compound of the invention to the enzyme. Accordingly, the ability of a test compound to compete with the radio-labeled compound for binding to the enzyme directly correlates to its binding affinity.

Kits

The present invention also includes pharmaceutical kits useful, for example, in the treatment or prevention of 11βHSD1-associated diseases or disorders, obesity, diabetes and other diseases referred to herein which include one or more containers containing a pharmaceutical composition comprising a therapeutically effective amount of a compound of the invention. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results. The compounds of the example section were found to be inhibitors or antagonists of 11βHSD1 or MR according to one or more of the assays provided herein.

### EXAMPLES

### Example 1

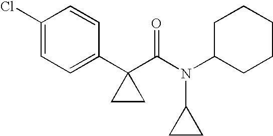

### 1-(4-Chlorophenyl)-N-cyclohexyl-N-cyclopropylcyclopropanecarboxamide

### Step 1. N-cyclopropylcyclohexanamine

1.21 mL of cyclopropylamine was mixed with 1.82 mL of cyclohexanone in 5.0 mL 1,2-dichloroethane, the reaction mixture was stirred at room temperature for 15 min, followed by the addition of 4.45 g of sodium triacetoxyborohydride. The reaction mixture was stirred overnight. The reaction mixture was then diluted with ethyl acetate. The organic layer was washed with saturated NaHCO₃, brine, dried and concentrated under vacuum to afford a residue, which was used directly in the next step. LCMS: (M+H)⁺=140.1.

### Step 2. 1-(4-Chlorophenyl)-N-cyclohexyl-N-cyclopropylcyclopropanecarboxamide

To a solution of 1-(4-chlorophenyl)cyclopropanecarboxylic acid (20 mg) and N-cyclopropylcyclohexanamine (17 mg) in 0.3 mL DMF was added 49.5 mg BOP coupling reagent. The pH of the reaction mixture was adjusted to about 9, and the resulting solution was stirred at room temperature for overnight. The reaction mixture was directly purified by HPLC to afford the desired product. LCMS: (M+H)⁺=318.1/320.1.

### Example 2

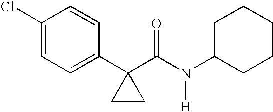

### 1-(4-Chlorophenyl)-N-cyclohexylcyclopropanecarboxamide

This compound was prepared using procedures analogous to those for example 1. LCMS: (M+H)⁺=278.0/280.0.

### Example 3

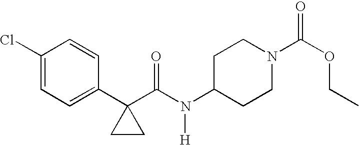

### Ethyl 4-(([1-(4-chlorophenyl)cyclopropyl]carbonyl}amino)piperidine-1-carboxylate

This compound was prepared using procedures analogous to those for example 1. LCMS: (M+H)⁺=351.1/353.1.

### Example 4

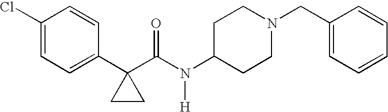

### N-(1-Benzylpiperidin-4-yl)-1-(4-chlorophenyl)cyclopropanecarboxamide

This compound was prepared using procedures analogous to those for example 1. LCMS: (M+H)⁺=369.1/371.0.

### Example 5

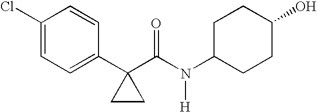

### 1-(4-Chlorophenyl)-N-(4-hydroxycyclohexyl)cyclopropanecarboxamide

This compound was prepared using procedures analogous to those for example 1. LCMS: (M+H)⁺=294.0/296.0.

### Example 6

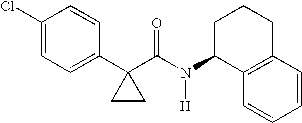

### 1-(4-Chlorophenyl)-N-[(1S)-1,2,3,4-tetrahydronaphthalen-1-yl]cyclopropanecarboxamide

This compound was prepared using procedures analogous to those for example 1. LCMS: (M+H)⁺=326.0/328.0.

### Example 7

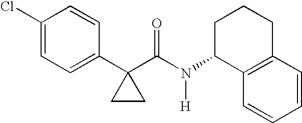

### 1-(4-Chlorophenyl)-N-[(1R)-1,2,3,4-tetrahydronaphthalen-1-yl]cyclopropanecarboxamide

This compound was prepared using procedures analogous to those for example 1. LCMS: (M+H)⁺=326.0/328.0.

### Example 7a

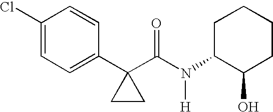

### 1-(4-Chlorophenyl)-N-[(1R,2R)-2-hydroxycyclohexyl]cyclopropanecarboxamide

This compound was prepared using procedures analogous to those for example 1. This compound was prepared using procedures analogous to those for example 1. LCMS: (M+H)⁺=294.0/296.0.

### Example 8

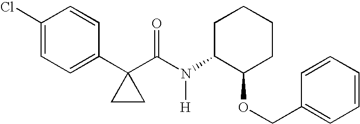

### N-[1(1R,2R)-2-(benzyloxy)cyclohexyl]-1-(4-chlorophenyl)cyclopropanecarboxamide

This compound was prepared using procedures analogous to those for example 1. LCMS: (M+H)⁺=384.1/386.1.

### Example 9

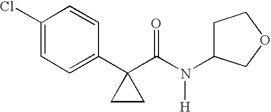

### 1-(4-Chlorophenyl)-N-(tetrahydrofuran-3-yl)cyclopropanecarboxamide

This compound was prepared using procedures analogous to those for example 1. LCMS: (M+H)⁺=266.0/267.9.

### Example 10

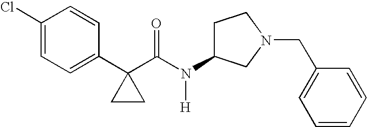

### N-[(3S)-1-benzylpyrrolidin-3-yl]-1-(4-chlorophenyl)cyclopropanecarboxamide

This compound was prepared using procedures analogous to those for example 1. LCMS: (M+H)⁺=355.0/357.1.

### Example 11

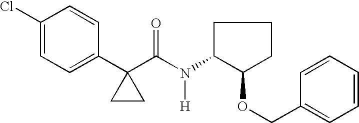

### N-[(1R,2R)-2-(benzyloxy)cyclopentyl]-1-(4-chlorophenyl)cyclopropanecarboxamide

This compound was prepared using procedures analogous to those for example 1. LCMS: (M+H)⁺=370.1/372.1.

### Example 12

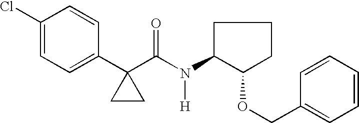

### N-[(1S,2S)-2-(benzyloxy)cyclopentyl]-1-(4-chlorophenyl)cyclopropanecarboxamide

This compound was prepared using procedures analogous to those for example 1. LCMS: (M+H)⁺=370.1/372.1.

### Example 13

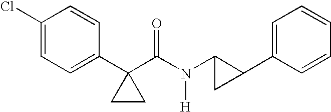

### 1-(4-Chlorophenyl)-N-(2-phenylcyclopropyl)cyclopropanecarboxamide

This compound was prepared using procedures analogous to those for example 1. LCMS: (M+H)⁺=312.0/314.0.

### Example 14

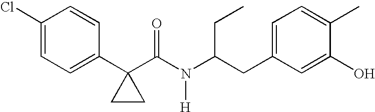

### 1-(4-Chlorophenyl)-N-[1-(3-hydroxy-4-methylbenzyl)propyl]cyclopropanecarboxamide

This compound was prepared using procedures analogous to those for example 1. LCMS: (M+H)⁺=358.1/360.1.

### Example 15

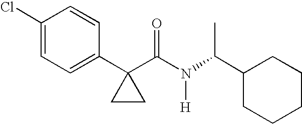

### 1-(4-Chlorophenyl)-N-[(1R)-1-cyclohexylethyl]cyclopropanecarboxamide

This compound was prepared using procedures analogous to those for example 1. LCMS: (M+H)⁺=306.0/308.0.

### Example 16

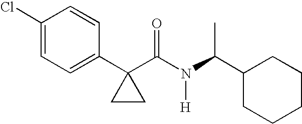

### 1-(4-Chlorophenyl)-N-[(1S)-1-cyclohexylethyl]cyclopropanecarboxamide

This compound was prepared using procedures analogous to those for example 1. LCMS: (M+H)⁺=306.0/308.0.

### Example 17

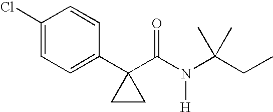

### 1-(4-Chlorophenyl)-N-(1,1-dimethylpropyl)cyclopropanecarboxamide

This compound was prepared using procedures analogous to those for example 1. LCMS: (M+H)⁺=266.0/268.0.

### Example 18

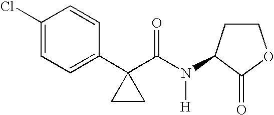

### 1-(4-Chlorophenyl)-N-[(3S)-2-oxotetrahydrofuran-3-yl]cyclopropanecarboxamide

This compound was prepared using procedures analogous to those for example 1. LCMS: (M+H)⁺=280.0/282.0.

### Example 19

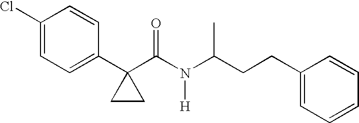

### 1-(4-Chlorophenyl)-N-(1-methyl-3-phenylpropyl)cyclopropanecarboxamide

This compound was prepared using procedures analogous to those for example 1. LCMS: (M+H)⁺=328.0/330.0.

### Example 20

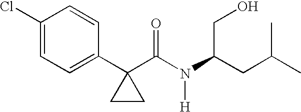

### 1-(4-Chlorophenyl)-N-[(1R)-1-(hydroxymethyl)-3-methylbutyl]-cyclopropanecarboxamide

This compound was prepared using procedures analogous to those for example 1. LCMS: (M+H)⁺=296.0/298.0.

### Example 21

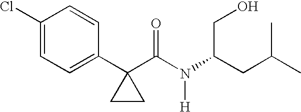

### 1-(4-Chlorophenyl)-N-[(1S)-1-(hydroxymethyl)-3-methylbutyl]-cyclopropanecarboxamide

This compound was prepared using procedures analogous to those for example 1. LCMS: (M+H)⁺=296.0/298.0.

### Example 22

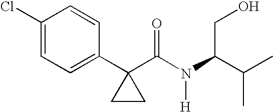

### 1-(4-Chlorophenyl)-N-[(1R)-1-(hydroxymethyl)-2-methylpropyl]-cyclopropanecarboxamide

This compound was prepared using procedures analogous to those for example 1. LCMS: (M+H)⁺=282.0/284.0.

### Example 23

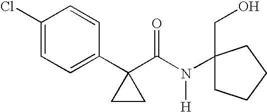

### 1-(4-Chlorophenyl)-N-[1-(hydroxymethyl)cyclopentyl]cyclopropanecarboxamide

This compound was prepared using procedures analogous to those for example 1. LCMS: (M+H)⁺=294.0/296.0.

### Example 24

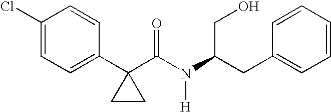

### N-[(1R)-1-benzyl-2-hydroxyethyl]-1-(4-chlorophenyl)cyclopropanecarboxamide

This compound was prepared using procedures analogous to those for example 1. LCMS: (M+H)⁺=330.0/332.0.

### Example 25

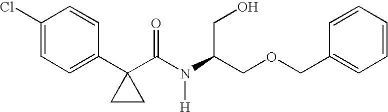

### N-[(1S)-2-(benzyloxy)-1-(hydroxymethyl)ethyl]-1-(4-chlorophenyl)cyclopropanecarboxamide

This compound was prepared using procedures analogous to those for example 1. LCMS: (M+H)⁺=360.0/362.0.

### Example 26

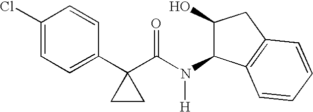

### 1-(4-Chlorophenyl)-N-[(1R,2S)-2-hydroxy-2,3-dihydro-1H-inden-1-yl]cyclopropanecarboxamide

This compound was prepared using procedures analogous to those for example 1. LCMS: (M+H)⁺=328.0/330.0.

### Example 27

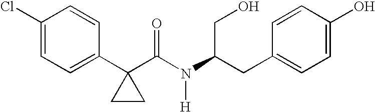

### 1-(4-Chlorophenyl)-N-[(1R)-2-hydroxy-1-(4-hydroxybenzyl)ethyl]cyclopropanecarboxamide

This compound was prepared using procedures analogous to those for example 1. LCMS: (M+H)⁺=346.1/348.0.

### Example 28

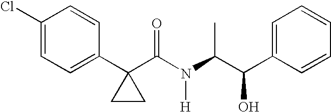

### 1-(4-chlorophenyl)-N-[(1S,2R)-2-hydroxy-1-methyl-2-phenylethyl]cyclopropanecarboxamide

This compound was prepared using procedures analogous to those for example 1. LCMS: (M+H)⁺=330.0/332.0; (M−H₂O+H)⁺=312.0/314.0.

### Example 29

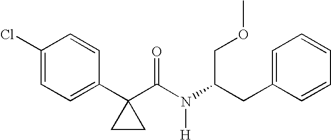

### N-[(1S)-1-benzyl-2-methoxyethyl]-1-(4-chlorophenyl)cyclopropanecarboxamide

This compound was prepared using procedures analogous to those for example 1. LCMS: (M+H)⁺=344.0/346.0.

### Example 30

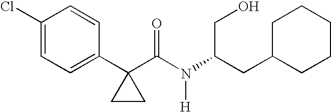

### 1-(4-Chlorophenyl)-N-[(1S)-2-cyclohexyl-1-(hydroxymethyl)ethyl]cyclopropanecarboxamide

This compound was prepared using procedures analogous to those for example 1. LCMS: (M+H)⁺=336.0/338.1.

### Example 31

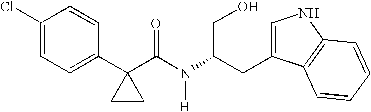

### 1-(4-Chlorophenyl)-N-[(1S)-2-hydroxy-1-(1H-indol-3-ylmethyl)ethyl]-cyclopropanecarboxamide

This compound was prepared using procedures analogous to those for example 1. LCMS: (M+H)⁺=369.0/371.0.

### Example 32

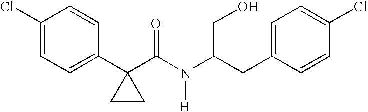

### N-[1-(4-Chlorobenzyl)-2-hydroxyethyl]-1-(4-chlorophenyl)cyclopropanecarboxamide

This compound was prepared using procedures analogous to those for example 1. LCMS: (M+H)⁺=364.0/366.0.

### Example 33

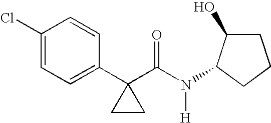

### 1-(4-Chlorophenyl)-N-[(1S,2S)-2-hydroxycyclopentyl]cyclopropanecarboxamide

This compound was prepared using procedures analogous to those for example 1. LCMS: (M+H)⁺=280.0/282.0.

### Example 34

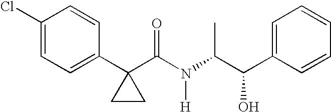

### 1-(4-Chlorophenyl)-N-[(1R,2S)-2-hydroxy-1-methyl-2-phenylethyl]-cyclopropanecarboxamide

This compound was prepared using procedures analogous to those for example 1. LCMS: (M+H)⁺=330.0/332.0; (M−H20 +H)⁺=312.0/314.0.

### Example 35

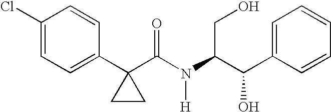

### 1-(4-Chlorophenyl)-N-[(1S,2S)-2-hydroxy-1-(hydroxymethyl)-2-phenylethyl]cyclopropanecarboxamide

This compound was prepared using procedures analogous to those for example 1. LCMS: (M+H)⁺=346.0/348.0.

### Example 36

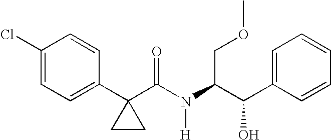

### 1-(4-Chlorophenyl)-N-[(1S,2S)-2-hydroxy-1-(methoxymethyl)-2-phenylethyl]-cyclopropanecarboxamide

This compound was prepared using procedures analogous to those for example 1. LCMS: (M+H)⁺=360.0/362.0.

### Example 37

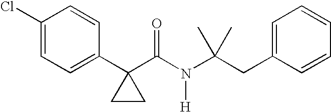

### 1-(4-Chlorophenyl)-N-(1,1-dimethyl-2-phenylethyl)cyclopropanecarboxamide

This compound was prepared using procedures analogous to those for example 1. LCMS: (M+H)⁺=328.0/330.0.

### Example 38

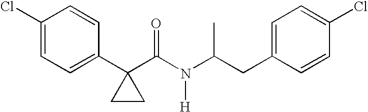

### 1-(4-chlorophenyl)-N-[2-(4-chlorophenyl)-1-methylethyl]cyclopropanecarboxamide

This compound was prepared using procedures analogous to those for example 1. LCMS: (M+H)⁺=347.9/350.0.

### Example 39

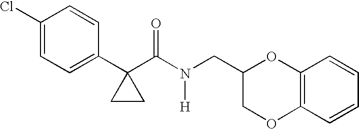

### 1-(4-Chlorophenyl)-N-(2,3-dihydro-1,4-benzodioxin-2-ylmethyl)cyclopropanecarboxamide

This compound was prepared using procedures analogous to those for example 1. LCMS: (M+H)⁺=344.0/346.0.

### Example 40

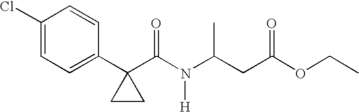

### Ethyl 3-({[1-(4-chlorophenyl)cyclopropyl]carbonyl}amino)butanoate

This compound was prepared using procedures analogous to those for example 1. LCMS: (M+H)⁺=310.0/312.0.

### Example 41

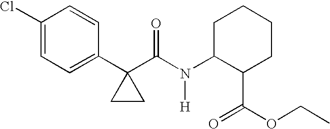

### Ethyl (cis)2-({[-(4-chlorophenyl)cyclopropyl]carbonyl}amino)cyclohexanecarboxylate

This compound was prepared using procedures analogous to those for example 1. LCMS: (M+H)⁺=350.0/352.0.

### Example 42

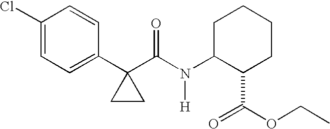

### Ethyl (trans)-2-({[1-(4-chlorophenyl)-cyclopropyl]carbonyl}amino)-cyclohexanecarboxylate

This compound was prepared using procedures analogous to those for example 1. LCMS: (M+H)⁺=350.0/352.0.

### Example 43

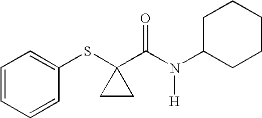

### N-Cyclohexyl-1-(phenylthio)cyclopropanecarboxamide

### Step 1. Methyl 1-(phenylthio)cyclopropanecarboxylate

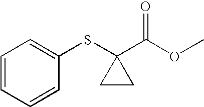

Sodium hydride (60% in mineral oil, 1.11 g, 27.8 mmol) was suspended in ether (30 mL) and cooled to 0° C. A premixed solution of 1,2-dibromoethane (2.56 mL, 29.67 mmol), methyl(phenylthio)acetate, ether (30 mL) and DMSO (10 mL) was added dropwise with vigorous stirring via cannula at 0° C. The reaction mixture was stirred at rt for 36 h, prior to quenching by the addition of water and EtOAc. After stirring for a few min., to dissolve all the solids, the layers were separated. The organic layer was washed with brine, dried over MgSO₄, filtered and concentrated. The residue was purified by flash chromatography (silica, hexanes:ether, 6:1 to 5:1 to 4:1) to provide the desired product, which was used in the subsequent step without further purification.

### Step 2. 1-(Phenylthio)cyclopropanecarboxylic acid

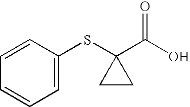

Methyl 1-(phenylthio)cyclopropanecarboxylate (1.04 g, 4.99 mmol) was dissolved in THF (18 mL) and MeOH (6 mL) and to this solution was added an aqueous solution of lithium hydroxide monohydrate (1.05 g, 25.0 mmol in 6 mL of water). After stirring at rt for 16 h, the volatiles were removed and the remaining aqueous solution was acidified to pH 2 with a 1 N HCl solution. Following extraction with EtOAc, the organic layer was dried over MgSO₄, filtered and concentrated to provide the desired carboxylic acid as a white solid (0.931 g, 96.0% yield).

### Step 3. N-Cyclohexyl-1-(phenylthio)cyclopropanecarboxamide

1-(Phenylthio)cyclopropanecarboxylic acid was converted to the final compound using procedures analogous to those described for the synthesis of example 1. LCMS: (M+H)⁺=276.0.

### Example 44

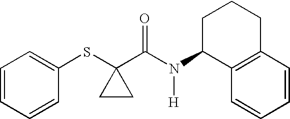

### 1-(phenylthio)-N-[(1S)-1,2,3,4-tetrahydronaphthalen-1-yl]cyclopropanecarboxamide

This compound was prepared using procedures analogous to those for example 43. LCMS: (M+H)⁺=324.0.

### Example 45

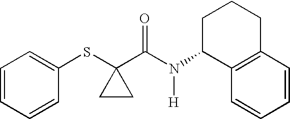

### 1-(phenylthio)-N-[(1R)-1,2,3,4-tetrahydronaphthalen-1-yl]cyclopropanecarboxamide

This compound was prepared using procedures analogous to those for example 43. LCMS: (M+H)⁺=324.0.

### Example 46

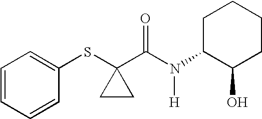

### N-[(1R,2R)-2-hydroxycyclohexyl]-1-(phenylthio)cyclopropanecarboxamide

This compound was prepared using procedures analogous to those for example 43. LCMS: (M+H)⁺=292.0.

### Example 47

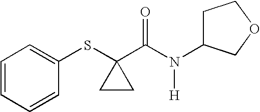

### 1-(phenylthio)-N-(tetrahydrofuran-3-yl)cyclopropanecarboxamide

This compound was prepared using procedures analogous to those for example 43. LCMS: (M+H)⁺=264.0.

### Example 48

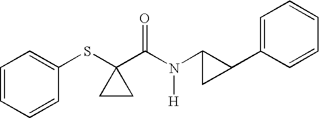

### N-(2-phenylcyclopropyl)-1-(phenylthio)cyclopropanecarboxamide

This compound was prepared using procedures analogous to those for example 43. LCMS: (M+H)⁺=310 0.

### Example 49

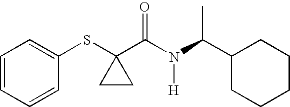

### N-[(1S)-1-cyclohexylethyl]-1-(phenylthio)cyclopropanecarboxamide

This compound was prepared using procedures analogous to those for example 43. LCMS: (M+H)⁺=304.1.

### Example 50

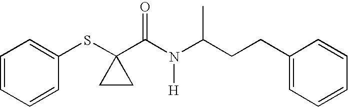

### N-(1-methyl-3-phenylpropyl)-1-(phenylthio)cyclopropanecarboxamide

This compound was prepared using procedures analogous to those for example 43. LCMS: (M+H)⁺=326.0.

### Example 51

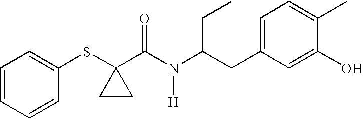

### N-[1-(3-hydroxy-4-methylbenzyl)propyl]-1-(phenylthio)cyclopropanecarboxamide

This compound was prepared using procedures analogous to those for example 43. LCMS: (M+H)⁺=356.0.

### Example 52

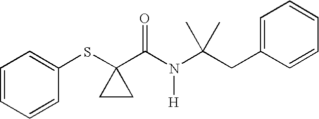

### N-(1,1-dimethyl-2-phenylethyl)-1-(phenylthio)cyclopropanecarboxamide

This compound was prepared using procedures analogous to those for example 43. LCMS: (M+H)⁺=326.0.

### Example 53

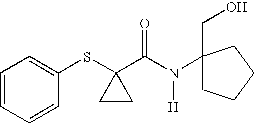

### N-[1-(hydroxymethyl)cyclopentyl]-1-(phenylthio)cyclopropanecarboxamide

This compound was prepared using procedures analogous to those for example 43. LCMS: (M+H)⁺=292.0.

### Example 54

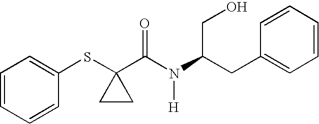

### N-[(1R)-1-benzyl-2-hydroxyethyl]-1-(phenylthio)cyclopropanecarboxamide

This compound was prepared using procedures analogous to those for example 43. LCMS: (M+H)⁺=328.0.

### Example 55

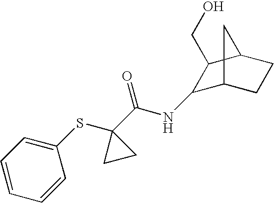

### N-[3-(hydroxymethyl)bicyclo[2.2.1]hept-2-yl]-1-(phenylthio)cyclopropanecarboxamide

This compound was prepared using procedures analogous to those for example 43. LCMS: (M+H)⁺=318.0.

### Example 56

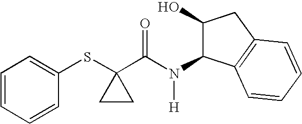

### N-[(1R,2S)-2-hydroxy-2,3-dihydro-1H-inden-1-yl]-1-(phenylthio)-cyclopropanecarboxamide

This compound was prepared using procedures analogous to those for example 43. LCMS: (M+H)⁺=326.0.

### Example 57

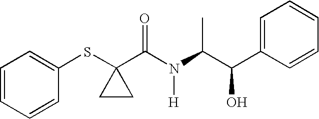

### N-[(1S,2R)-2-hydroxy-1-methyl-2-phenylethyl]-1-(phenylthio)cyclopropanecarboxamide

This compound was prepared using procedures analogous to those for example 43. LCMS: (M+H)⁺=328.0; (M−H₂O+H)⁺=310.0.

### Example 58

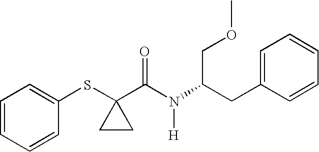

### N-[(1S)-1-benzyl-2-methoxyethyl]-1-(phenylthio)cyclopropanecarboxamide

This compound was prepared using procedures analogous to those for example 43. LCMS: (M+H)⁺=342.1.

### Example 59

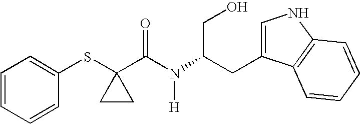

### N-[(1S)-2-hydroxy-1-(1H-indol-3-ylmethyl)ethyl]-1-(phenylthio)-cyclopropanecarboxamide

This compound was prepared using procedures analogous to those for example 43. LCMS: (M+H)⁺=367.0.

### Example 60

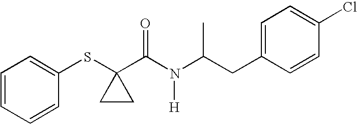

### N-[2-(4-Chlorophenyl)-1-methylethyl]-1-(phenylthio)cyclopropanecarboxamide

This compound was prepared using procedures analogous to those for example 43. LCMS: (M+H)⁺=346.0/348.0.

### Example 61

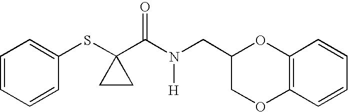

### N-(2,3-Dihydro-1,4-benzodioxin-2-ylmethyl)-1-(phenylthio)cyclopropanecarboxamide

This compound was prepared using procedures analogous to those for example 43. LCMS: (M+H)⁺=342.0.

### Example 62

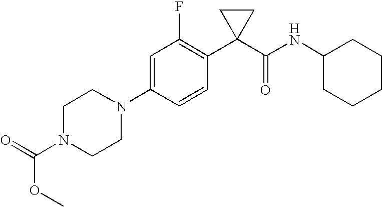

### Methyl 4-(4-{1-[1(cyclohexylamino)carbonyl]cyclopropyl}-3-fluorophenyl)piperazine-1-carboxylate

### Step 1. 1-(4-Bromo-2-fluorophenyl)cyclopropanecarboxylic acid

Sodium hydroxide, 50% aqueous solution (5.71 mL, 0.149 mol), was added to a mixture of (4-bromo-2-fluorophenyl)acetonitrile (3.16 g, 0.0145 mol), benzyltriethylammonium chloride (0.26 g, 0.0011 mol), and 1-bromo-2-chloro-ethane (2.51 mL, 0.0302 mol) at 50° C. for 10 h. The mixture was poured into ice-water (50 mL) and was extracted with ethyl ether (2×50 mL). The combined organic phase was washed with brine (30 mL), dried over MgSO₄, filtered, and concentrated under reduced pressure to give 2.88 g of brown solid. ¹HNMR confirmed that desired nitrile intermediate was isolated. To the resulting residue was added 50% NaOH aqueous solution (3.8 mL) and ethylene glycol (20 mL) and the solution was heated to 100° C. and stirred overnight. The reaction mixture was poured into 50 mL of water and washed with ether (2×50 mL). The aqueous layer was cooled with an ice bath and then acidified by the slow addition of 6 N HCl. to pH=2. The product was extracted with EtOAc (2×100 mL), dried over MgSO₄ and concentrated to give 1.634 g. (70%) of the desired product. ¹H NMR confirmed that the desired product was isolated.

### Step 2. 1-{4-[4-(tert-Butoxycarbonyl)piperazin-1-yl]-2-fluorophenyl}cyclopropane carboxylic acid

A mixture of 1-(4-bromo-2-fluorophenyl)cyclopropanecarboxylic acid (5.0 g, 0.019 mol), tert-butyl piperazine-1-carboxylate (4.3 g, 0.023 mol), sodium tert-butoxide (4.4 g, 0.046 mol), palladium acetate (100 mg, 0.0006 mol) and 2-(di-t-butylphosphino)biphenyl (200 mg, 0.0006 mol) was evacuated and then charged with nitrogen. To the mixture was added 1,4-dioxane (60 mL, 0.8 mol) and the resulting mixture was refluxed overnight. The reaction mixture was poured into cold saturated. NH₄Cl and then extracted with ethyl acetate and the combined extracts were washed with brine, dried, and concentrated. The product was purified by CombiFlash using 6% methanol in methylene chloride. LCMS: (M-t-Bu+H)=309.1.

### Step 3. tert-Butyl 4-(4-{1-[(cyclohexylamino)carbonyl]cyclopropyl)-3-fluorophenyl)piperazine-1-carboxylate

The title compound was prepared by using a procedure that was analogous to that used for the synthesis of example 1, step 2.

### Step 4. N-Cyclohexyl-]-(2-fluoro-4-piperazin-1-ylphenyl)cyclopropanecarboxamide hydrochloride

tert-Butyl 4-(4-{1-[(cyclohexylamino)carbonyl]cyclopropyl}-3-fluorophenyl)-piperazine-1-carboxylate was dissolved in 4.0 M HCl in 1,4-dioxane and the reaction mixture was stirred at rt for 2 h. The volatiles were removed and the residue was used in the next step without further purification.

### Step 5. Methyl 4-[3-fluoro-4-(1-{[(trans-4-hydroxycyclohexyl)amino]carbonyl}cyclopropyl)phenyl]piperazine-1-carboxylate

Methyl chloroformate (5.4 μL, 0.000069 mol) was added to a mixture of N-cyclohexyl-1-(2-fluoro-4-piperazin-1-ylphenyl)cyclopropanecarboxamide hydrochloride (20 mg, 0.00006 mol) and triethylamine (25 μL, 0.00018 mol) in dichloromethane (0.5 mL) and the resulting solution was stirred at rt for 1 h. The crude product was purified by prep-HPLC to afford the desired product. LCMS: (M+H)⁺=404.2.

### Example 63

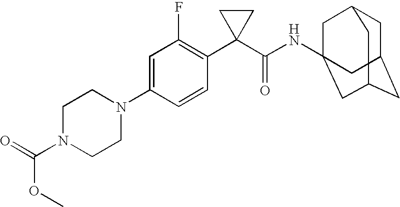

### Methyl 4-(4-{1-[(1-adamantylamino)carbonyl]cyclopropyl)-3-fluorophenyl)piperazine-1-carboxylate

This compound was prepared by using a procedure that was analogous to that used for the synthesis of example 62. LCMS: (M+H)⁺=456.2.

### Example 64

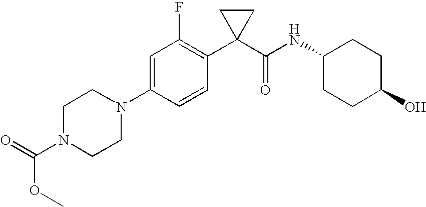

### Methyl 4-[3-fluoro-4-(1-{[(trans-4-hydroxycyclohexyl)amino]carbonyl}cyclopropyl) phenyl]piperazine-1-carboxylate

This compound was prepared by using a procedure that was analogous to that used for the synthesis of example 62. LCMS: (M+H)⁺=420.2.

### Example 65

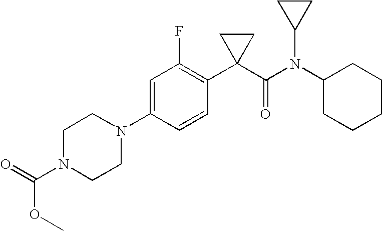

### Methyl 4-[4-(1-{[cyclohexyl(cyclopropyl)amino]carbonyl}cyclopropyl)-3-fluorophenyl]piperazine-1-carboxylate

This compound was prepared by using a procedure that was analogous to that used for the synthesis of example 62. LCMS: (M+H)⁺=444.2.

### Example 66

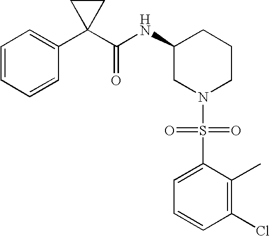

### N-{1-[(3-Chloro-2-methylphenyl)sulfonyl]piperidin-3-yl}-1-phenylcyclopropane carboxamide

### Step 1. tert-Butyl ((3S)-1-[(3-chloro-2-methylphenyl)sulfonyl]piperidin-3-ylycarbamate

A solution of 3-chloro-2-methylbenzenesulfonyl chloride (0.75 g, 0.0033 mol) in 5 ml of acetonitrile was added into a solution of tert-butyl (3S)-piperidin-3-ylcarbamate (0.67 g, 0.0033 mol) in 5 ml of acetonitrile at 0° C. After stirring at rt for 1.5 h, the reaction mixture was filtered and concentrated to give a crude product, which was used in the next step without further purification.

### Step 2. (3S)-1-[(3-Chloro-2-methylphenyl)sulfonyl]piperidin-3-amine hydrochloride

4.0 M of HCl in 1,4-dioxane (4 ml) was added to tert-butyl {(3S)-1-[(3-chloro-2-methylphenyl)sulfonyl]piperidin-3-yl}carbamate (3.3 mmol, 0.0033 mol). After stirring at rt for 1 hr, the reaction mixture was concentrated to give the desired product, which was used in the next step without further purification.

### Step 3.

The title compound was prepared by using a procedure that was analogous to that used for the synthesis of example 1. LCMS: (M+H)⁺=433.1.

### Example 67

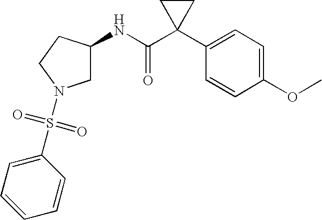

### 1-(4-Methoxyphenyl)-N-[(3R)-1-(phenylsulfonyl)pyrrolidin-3-yl]cyclopropane carboxamide

### Step 1. (3R)-1-(phenylsulfonyl)pyrrolidin-3-amine hydrochloride

Benzenesulfonyl chloride (91.0 mg, 0.000515 mol) was added to a mixture of tert-butyl (3R)-pyrrolidin-3-ylcarbamate (95.0 mg, 0.000510 mol) and potassium carbonate (150 mg, 0.0011 mol) in acetonitrile (3.0 mL, 0.057 mol) at rt. After stirring for 1 h, the reaction mixture was filtered. The filtrate was concentrated under reduced pressure and the residue was treated with 4.0 M of hydrogen chloride in 1,4-dioxane (2.0 mL) at rt for 1 h. The solvent was evaporated under reduced pressure to give the desired product, which was used in next step without further purification.

### Step 2.

The title compound was prepared by using a procedure that was analogous to that used for the synthesis of example 1. LCMS: (M+H)⁺=401.1.

### Example 68

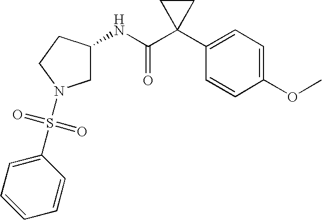

### 1-(4-Methoxyphenyl)-N-(3S)-1-(phenylsulfonyl)pyrrolidin-3-yl]cyclopropane carboxamide

This compound was prepared by using a procedure that was analogous to that used for the synthesis of example 67. LCMS: (M+H)⁺=401.1.

### Example 69

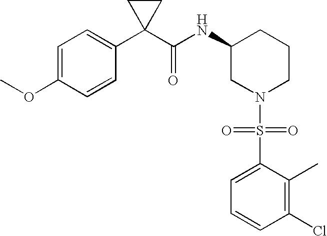

### N-{(3S)-1-[(3-Chloro-2-methylphenyl)sulfonyl]piperidin-3-yl}-1-(4-methoxyphenyl) eyclopropanecarboxamide

This compound was prepared by using a procedure that was analogous to that used for the synthesis of example 66. LCMS: (M+H)⁺=463.1.

### Example 70

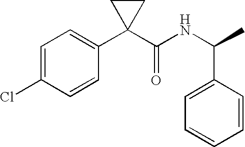

### 1-(4-Chlorophenyl)-N-[(1S)-1-phenylethyl]cyclopropanecarboxamide

This compound was prepared by using a procedure that was analogous to that used for the synthesis of example 1. LCMS: (M+H)⁺=300.1.

### Example 71

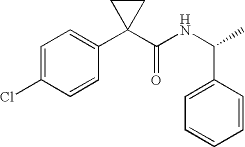

### 1-(4-Chlorophenyl)-N-[(1R)-1-phenylethyl]cyclopropanecarboxamide

This compound was prepared by using a procedure that was analogous to that used for the synthesis of example 1. LCMS: (M+H)⁺=300.1.

### Example 72

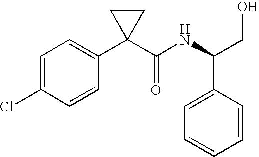

### 1-(4-Chlorophenyl)-N-[(1R)-2-hydroxy-1-phenylethyl]cyclopropanecarboxamide

This compound was prepared by using a procedure that was analogous to that used for the synthesis of example 1. LCMS: (M+H)⁺=316.3.

### Example 73

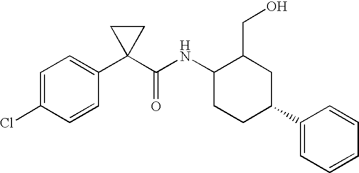

### 1-(4-Chlorophenyl)-N-[(4S)-2-(hydroxymethyl)-4-phenylcyclohexyl]cyclopropane carboxamide

This compound was prepared by using a procedure that was analogous to that used for the synthesis of example 1. LCMS: (M+H)⁺=384.2.

### Example 74

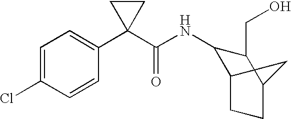

### 1-(4-Chlorophenyl)-N-[3-(hydroxymethyl)bicyclo[2.2.1]hept-2-yl]cyclopropane carboxamide

This compound was prepared by using a procedure that was analogous to that used for the synthesis of example 1. LCMS: (M+H)⁺=320.2.

### Example 75

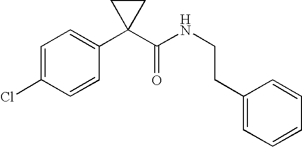

### 1-(4-Chlorophenyl)-N-(2-phenylethyl)cyclopropanecarboxamide

This compound was prepared by using a procedure that was analogous to that used for the synthesis of example 1. LCMS: (M+H)⁺=300.3.

### Example 76

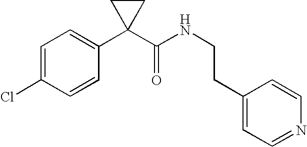

### 1-(4-Chlorophenyl)-N-(2-pyridin-4-ylethyl)cyclopropanecarboxamide

This compound was prepared by using a procedure that was analogous to that used for the synthesis of example 1. LCMS: (M+H)⁺=301.3.

### Example 77

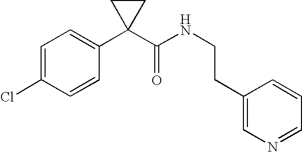

### 1-(4-Chlorophenyl)-N-(2-pyridin-3-ylethyl)cyclopropanecarboxamide

This compound was prepared by using a procedure that was analogous to that used for the synthesis of example 1. LCMS: (M+H)⁺=301.3.

### Example 78

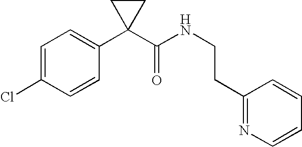

### 1-(4-Chlorophenyl)-N-(2-pyridin-2-ylethyl)cyclopropanecarboxamide

This compound was prepared by using a procedure that was analogous to that used for the synthesis of example 1. LCMS: (M+H)⁺=301.3.

### Example 79

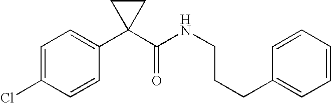

### 1-(4-Chlorophenyl)-N-(3-phenylpropyl)cyclopropanecarboxamide

This compound was prepared by using a procedure that was analogous to that used for the synthesis of example 1. LCMS: (M+H)⁺=314.3.

### Example 80

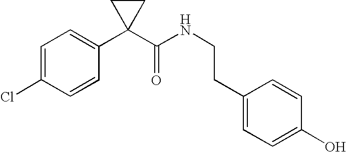

### 1-(4-Chlorophenyl)-N-[2-(4-hydroxyphenyl)ethyl]cyclopropanecarboxamide

This compound was prepared by using a procedure that was analogous to that used for the synthesis of example 1. LCMS: (M+H)⁺=316.3.

### Example 81

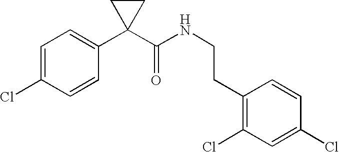

### 1-(4-Chlorophenyl)-N-[2-(2,4-dichlorophenyl)ethyl]cyclopropanecarboxamide

This compound was prepared by using a procedure that was analogous to that used for the synthesis of example 1. LCMS: (M+H)⁺=368.2 & 370.2.

### Example 82

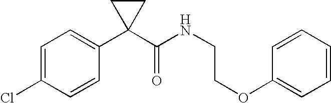

### 1-(4-Chlorophenyl)-N-(2-phenoxyethyl)cyclopropanecarboxamide

This compound was prepared by using a procedure that was analogous to that used for the synthesis of example 1. LCMS: (M+H)⁺=316.3.

### Example 83

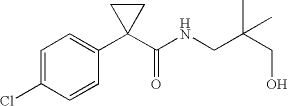

### 1-(4-Chlorophenyl)-N-(3-hydroxy-2,2-dimethylpropyl)cyclopropanecarboxamide

This compound was prepared by using a procedure that was analogous to that used for the synthesis of example 1. LCMS: (M+H)⁺=282.3.

### Example 84

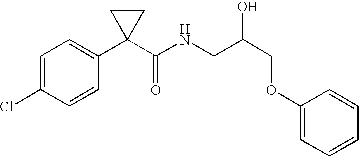

### 1-(4-Chlorophenyl)-N-(2-hydroxy-3-phenoxypropyl)cyclopropanecarboxamide

This compound was prepared by using a procedure that was analogous to that used for the synthesis of example 1. LCMS: (M+H)⁺=346.4.

### Example 85

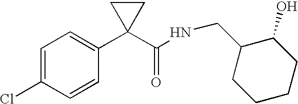

### 1-(4-Chlorophenyl)-N-{[(2R)-2-hydroxycyclohexyl]methyl}cyclopropanecarboxamide

This compound was prepared by using a procedure that was analogous to that used for the synthesis of example 1. LCMS: (M+H)⁺=308.4.

### Example 86

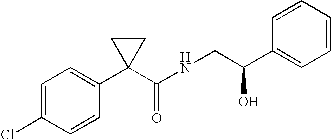

### 1-(4-Chlorophenyl)-N-[(2R)-2-hydroxy-2-phenylethyl]cyclopropanecarboxamide

This compound was prepared by using a procedure that was analogous to that used for the synthesis of example 1. LCMS: (M+H)⁺=316.4.

### Example 87

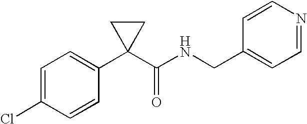

### 1-(4-Chlorophenyl)-N-(pyridin-4-ylmethyl)cyclopropanecarboxamide

This compound was prepared by using a procedure that was analogous to that used for the synthesis of example 1. LCMS: (M+H)⁺=287.2.

### Example 88

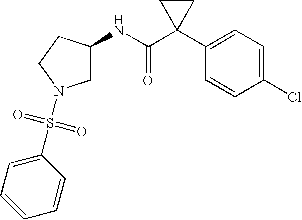

### 1-(4-Chlorophenyl)-N-[(3R)-1-(phenylsulfonyl)pyrrolidin-3-yl]cyclopropanecarboxamide

This compound was prepared by using a procedure that was analogous to that used for the synthesis of example 67. LCMS: (M+H)⁺=405.4.

### Example 89

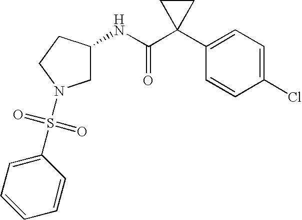

### 1-(4-Chlorophenyl)-N-[(3S)-1-(phenylsulfonyl)pyrrolidin-3-yl]cyclopropanecarboxamide

This compound was prepared by using a procedure that was analogous to that used for the synthesis of example 67. LCMS: (M+H)⁺=405.4.

### Example 90

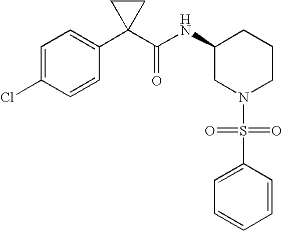

### 1-(4-Chlorophenyl)-N-[(3S)-1-(phenylsulfonyl)piperidin-3-yl]cyclopropanecarboxamide

This compound was prepared by using a procedure that was analogous to that used for the synthesis of example 66. LCMS: (M+H)⁺=419.4.

### Example 91

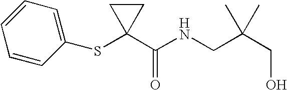

### N-(3-Hydroxy-2,2-dimethylpropyl)-1-(phenylthio)cyclopropanecarboxamide

This compound was prepared by using a procedure that was analogous to that used for the synthesis of example 43. LCMS: (M+H)⁺=280.1.

### Example 92

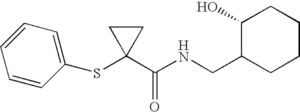

### N-{[(2R)-2-Hydroxycyclohexyl]methyl)-1-(phenylthio)cyclopropanecarboxamide

This compound was prepared by using a procedure that was analogous to that used for the synthesis of example 43. LCMS: (M+H)⁺=306.1.

### Example 93

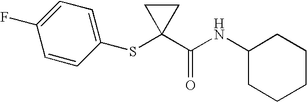

### N-Cyclohexyl-1-[(4-fluorophenyl)thio]cyclopropanecarboxamide

This compound was prepared by using a procedure that was analogous to that used for the synthesis of example 43. LCMS: (M+H)⁺=294.1.

### Example 94

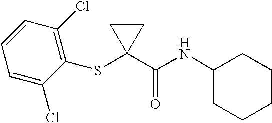

### N-Cyclohexyl-1-[(2,6-dichlorophenyl)thio]cyclopropanecarboxamide

This compound was prepared by using a procedure that was analogous to that used for the synthesis of example 43. LCMS: (M+H)⁺=345.1.

### Example 95

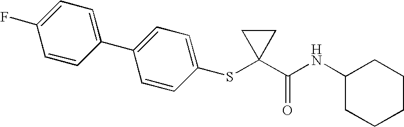

### N-Cyclohexyl-1-[(4′-fluorobiphenyl-4-yl)thio]cyclopropanecarboxamide

This compound was prepared by using a procedure that was analogous to that used for the synthesis of example 43. LCMS: (M+H)⁺=370.2.

### Example 96

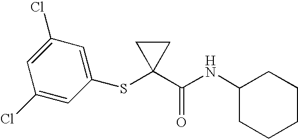

### N-Cyclohexyl-1-[(3,5-dichlorophenyl)thio]cyclopropanecarboxamide

This compound was prepared by using a procedure that was analogous to that used for the synthesis of example 43. LCMS: (M+H)⁺=345.1.

### Example 97

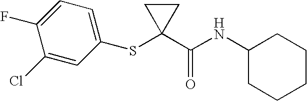

### 1-[(3-Chloro-4-fluorophenyl)thio]-N-cyclohexylcyclopropanecarboxamide

This compound was prepared by using a procedure that was analogous to that used for the synthesis of example 43. LCMS: (M+H)⁺=328.4.

### Example 98

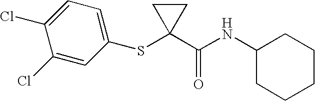

### N-Cyclohexyl-1-[(3,4-dichlorophenyl)thio]cyclopropanecarboxamide

This compound was prepared by using a procedure that was analogous to that used for the synthesis of example 43. LCMS: (M+H)⁺=345.1.

### Example 99

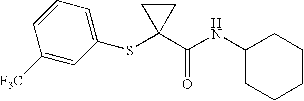

### N-Cyclohexyl-1-{[3-(trifluoromethyl)phenyl]thio}cyclopropanecarboxamide

This compound was prepared by using a procedure that was analogous to that used for the synthesis of example 43. LCMS: (M+H)⁺=344.1.

### Example 100

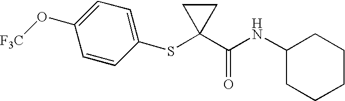

### N-Cyclohexyl-1-{[4-(trifluoromethoxy)phenyl]thio}cyclopropanecarboxamide

This compound was prepared by using a procedure that was analogous to that used for the synthesis of example 43. LCMS: (M+H)⁺=360.1.

### Example 101

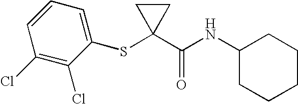

### N-Cyclohexyl-1-[(2,3-dichlorophenyl)thio]cyclopropanecarboxamide

This compound was prepared by using a procedure that was analogous to that used for the synthesis of example 43. LCMS: (M+H)⁺=345.1.

### Example 102

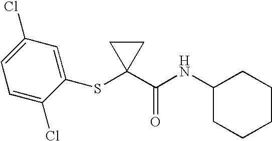

### N-Cyclohexyl-1-[(2,5-dichlorophenyl)thio]cyclopropanecarboxamide

This compound was prepared by using a procedure that was analogous to that used for the synthesis of example 43. LCMS: (M+H)⁺=345.1.

### Example 103

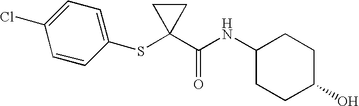

### 1-[(4-Chlorophenyl)thio]-N-(4-hydroxycyclohexyl)cyclopropanecarboxamide

This compound was prepared by using a procedure that was analogous to that used for the synthesis of example 43. LCMS: (M+H)⁺=326.4.

### Example 104

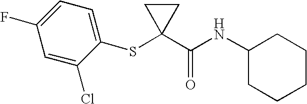

### 1-[(2-Chloro-4-fluorophenyl)thio]-N-cyclohexylcyclopropanecarboxamide

This compound was prepared by using a procedure that was analogous to that used for the synthesis of example 43. LCMS: (M+H)⁺=328.4.

### Example 105

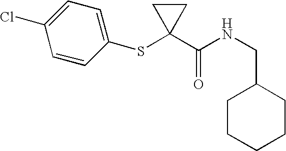

### 1-[(4-Chlorophenyl)thio]-N-(cyclohexylmethyl)cyclopropanecarboxamide

This compound was prepared by using a procedure that was analogous to that used for the synthesis of example 43. LCMS: (M+H)⁺=324.4.

### Example 106

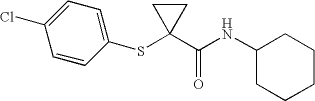

### 1-[(4-Chlorophenyl)thio]-N-cyclohexylcyclopropanecarboxamide

This compound was prepared by using a procedure that was analogous to that used for the synthesis of example 43. LCMS: (M+H)⁺=310.4.

### Example 107

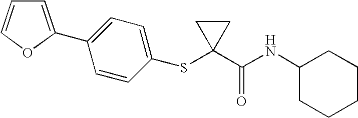

### N-Cyclohexyl-1-{[4-(2-furyl)phenyl]thio}cyclopropanecarboxamide

This compound was prepared using a procedure that was analogous to that used for the synthesis of example 43. LCMS: (M+H)⁺=342.2.

### Example 108

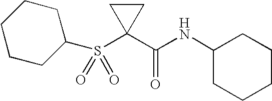

### N-Cyclohexyl-1-(cyclohexylsulfonyl)cyclopropanecarboxamide

### Step 1. Ethyl (cyclohexylsulfonyl)acetate

A solution of ethyl(cyclohexylthio)acetate in methylene chloride was added to a solution of m-chloroperbenzoic acid in methylene chloride (25 mL) at 0° C. The resulting solution was stirred at rt overnight. The volatiles were removed in-vacuo. The resulting residue was dissolved in CHCl₃ and washed with saturated NaHCO₃ and saturated Na₂S₂O₃ The organic layer was dried over MgSO₄ and concentrated in-vacuo and the crude residue was purified by flash chromatography, eluting with hexane/EtOAc (3:1, 2:1, 1:1) to give 0.53 g of the desired product as a colorless oil, which was identified by ¹H NMR as the desired product.

### Step 2. N-Cyclohexyl-1-(cyclohexylsulfonyl)cyclopropanecarboxamide

The title compound was prepared by using a procedure that was analogous to that used for the synthesis of example 43. LCMS: (M+H)⁺=314.2.

### Example 109

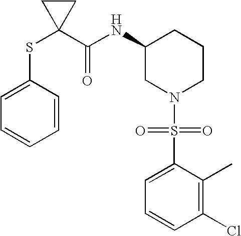

### N-{(3S)-1-[(3-chloro-2-methylphenyl)sulfonyl]piperidin-3-yl}-1-(phenylthio) cyclopropanecarboxamide

This compound was prepared by using a procedure that was analogous to that used for the synthesis of example 66, steps 1 & 2 and example 43, steps 1-3. LCMS: (M+H)⁺=465.1.

### Example 110

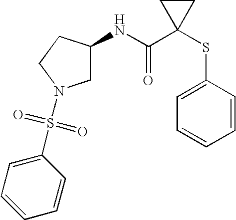

### N-[(3R)-1-(phenylsulfonyl)pyrrolidin-3-yl]-1-(phenylthio)cyclopropanecarboxamide

This compound was prepared by using a procedure that was analogous to that used for the synthesis of example 67, steps 1 and example 43, steps 1-3. LCMS: (M+H)⁺=403.2.

### Example 111

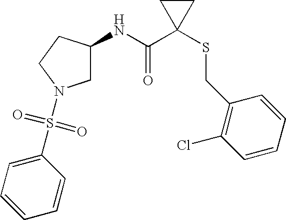

### 1-[(2-Chlorobenzyl)thiol-N-[(3R)-1-(phenylsulfonyl)pyrrolidin-3-yl]cyclopropane carboxamide

This compound was prepared by using a procedure that was analogous to that used for the synthesis of example 67, steps 1 and example 43, steps 1-3. LCMS: (M+H)⁺=452.0.

### Example 112

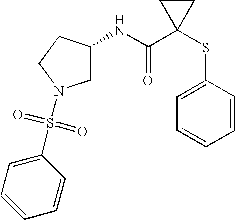

### N-[(3S)-1-(Phenylsulfonyl)pyrrolidin-3-yl]-1-(phenylthio)cyclopropanecarboxamide

This compound was prepared by using a procedure that was analogous to that used for the synthesis of example 67, steps 1 and example 43, steps 1-3. LCMS: (M+H)⁺=403.2.

### Example 113

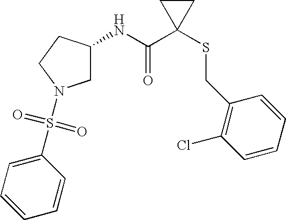

### 1-[(2-Chlorobenzyl)thiol-N-[(3S)-1-(phenylsulfonyl)pyrrolidin-3-yl]cyclopropane carboxamide

This compound was prepared by using a procedure that was analogous to that used for the synthesis of example 67, steps 1 and example 43, steps 1-3. LCMS: (M+H)⁺=452.0.

### Example 114

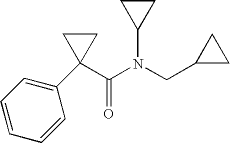

### N-cyclopropyl-N-(cyclopropylmethyl)-1-phenylcyclopropanecarboxamide

This compound was prepared by using a procedure that was analogous to that used for the synthesis of example 1. LCMS: (M+H)⁺=256.1.

### Example 115

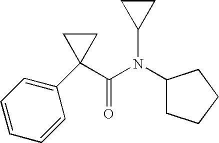

### N-cyclopentyl-N-cyclopropyl-1-phenylcyclopropanecarboxamide

This compound was prepared by using a procedure that was analogous to that used for the synthesis of example 1. LCMS: (M+H)⁺=270.1.

### Example 116

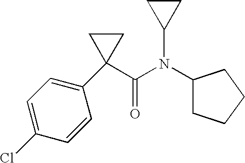

### 1-(4-Chlorophenyl)-N-cyclopentyl-N-cyclopropylcyclopropanecarboxamide

This compound was prepared by using a procedure that was analogous to that used for the synthesis of example 1. LCMS: (M+H)⁺=304.3.

### Example 117

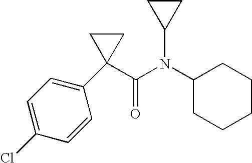

### 1-(4-Chlorophenyl)-N-cyclohexyl-N-cyclopropylcyclopropanecarboxamide

This compound was prepared by using a procedure that was analogous to that used for the synthesis of example 1. LCMS: (M+H)⁺=318.3.

### Example 118

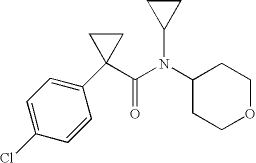

### 1-(4-Chlorophenyl)-N-cyclopropyl-N-(tetrahydro-2H-pyran-4-yl)cyclopropane carboxamide

This compound was prepared by using a procedure that was analogous to that used for the synthesis of example 1. LCMS: (M+H)⁺=320.4.

### Example 119

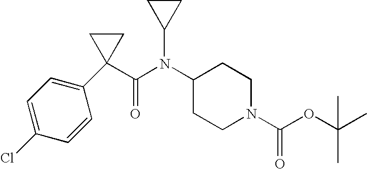

### tert-Butyl 4-[{[1-(4-chlorophenyl)cyclopropyl]carbonyl}(cyclopropyl)amino]piperidine-1-carboxylate

This compound was prepared by using a procedure that was analogous to that used for the synthesis of example 1. LCMS: (M+H)⁺=419.5.

### Example 120

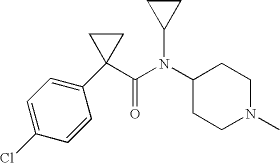

### 1-(4-Chlorophenyl)-N-cyclopropyl-N-(1-methylpiperidin-4-yl)cyclopropanecarboxamide

This compound was prepared by using a procedure that was analogous to that used for the synthesis of example 1. LCMS: (M+H)⁺=333.4

### Example 121

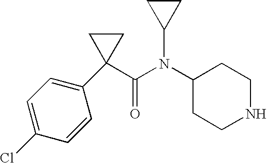

### 1-(4-Chlorophenyl)-N-cyclopropyl-N-piperidin-4-ylcyclopropanecarboxamide trifluoroacetate

tert-Butyl 4-[{[1-(4-chlorophenyl)cyclopropyl]carbonyl}(cyclopropyl)amino]piperidine-1-carboxylate was dissolved in methylene chloride (prepared according to example 119) and was treated with TFA at RT for 2 h. The reaction mixture was concentrated in vacuo and the resulting residue was purified by prep-HPLC. and LCMS to afford the desired product, which was confirmed by ¹H NMR and LCMS: M+H=319.

### Example 122

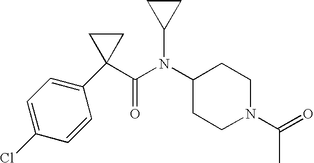

### N-(1-acetylpiperidin-4-yl)-1-(4-chlorophenyl)-N-cyclopropylcyclopropanecarboxamide

1-(4-Chlorophenyl)-N-cyclopropyl-N-piperidin-4-ylcyclopropanecarboxamide trifluoroacetate (prepared according to example 123) was dissolved in methylene chloride and to this was added DIEA and acetyl chloride. After stirring at rt for 2 h, the reaction mixture was poured into saturated NH₄Cl and extracted with CH₂Cl₂, washed with water, dried over MgSO₄, and concentrated in vacuo. The crude residue was purified by prep-HPLC to afford the desired product. The structure was confirmed by ¹H NMR and LCMS (M+H)=361.

### Example 123

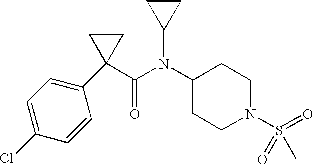

### 1-(4-Chlorophenyl)-N-cyclopropyl-N-[1-(methylsulfonyl)piperidin-4-yl]cyclopropanecarboxamide

This compound was prepared by using a procedure that was analogous to that used for the synthesis of example 122. LCMS: (M+H)⁺=397.

### Example 124

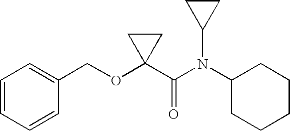

### 1-(Benzyloxy)-N-cyclohexyl-N-cyclopropylcyclopropanecarboxamide

### Step 1. Methyl 1-(benzyloxy)cyclopropanecarboxylate

At 0° C., methyl 1-hydoxycycloprppanecarboxylate was added to a suspension of NaH and DMF. After stirring for 10 min., benzylbromide was added and the reaction mixture was allowed to gradually warm to rt while stirring overnight. The reaction mixture was poured into ice water and extracted with ether (3×100 mL). The combined organic layers were washed with brine, dried over MgSO₄, and concentrated in-vacuo. The crude product was purified by flash chromatography, eluting with hexane/ether (3:1, 2:1, 1:1, 1:2) to give 600 mg of yellow oil. ¹H NMR confirmed the structure of the isolated product.

### Step 2. 1-(Betizyloxy)cyclopropanecarboxylic acid

Methyl 1-(benzyloxy)cyclopropanecarboxylate was dissolved in THF/MeOH and treated with an aq. solution of lithium hydroxide monohydrate. After stirring for 3 h, the volatiles were removed in-vacuo and the remaining aq. solution was acidified with 1 N HCl to pH 2. EtOAc was added and the layers were separated. The organic layer was dried over MgSO₄, filtered, and concentrated to provide the desired carboxylic acid as a pale yellow oil. ¹H NMR confirmed the isolated product.

### Step 3.

The title compound was prepared by using a procedure that was analogous to that used for the synthesis of example 1. LCMS: (M+H)⁺=314.1.

### Example 125

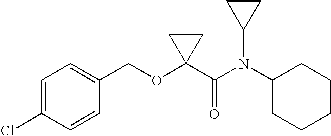

### 1-[(4-Chlorobenzyl)oxyl-N-cyclohexyl-N-cyclopropylcyclopropanecarboxamide

This compound was prepared by using a procedure that was analogous to that used for the synthesis of example 124, with the exception that the steps were reversed, such that the amide coupling was conducted prior to the alkylation of the alcohol. The product structure was confirmed by ¹H NMR and LCMS: (M+H)⁺=348.4.

### Example 126

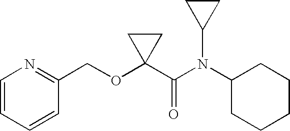

### N-Cyclohexyl-N-cyclopropyl-1-(pyridin-2-ylmethoxy)cyclopropanecarboxamide

This compound was prepared by using a procedure that was analogous to that used for the synthesis of example 125. The product structure was confirmed by ¹H NMR and LCMS: (M+H)⁺=315.1.

### Example 127

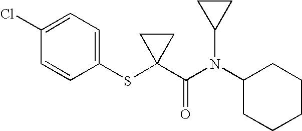

### 1-[(4-Chlorophenyl)thio]-N-cyclohexyl-N-cyclopropylcyclopropanecarboxamide

This compound was prepared by using a procedure that was analogous to that used for the synthesis of example 43. The product structure was confirmed by ¹H NMR and LCMS: (M+H)⁺=350.3.

### Example 128

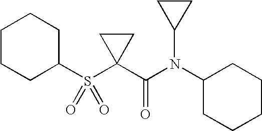

### N-Cyclohexyl-1-(cyclohexylsulfonyl)-N-cyclopropylcyclopropanecarboxamide

This compound was prepared by using a procedure that was analogous to that used for the synthesis of example 108. The product structure was confirmed by ¹HNMR and LCMS: (M+H)⁺=354.1.

### Example 129

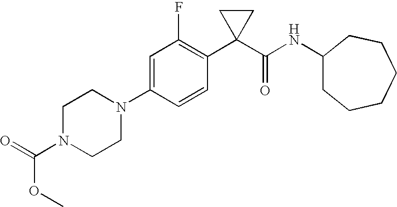

### Methyl 4-(4-{1-[(cycloheptylamino)carbonyl]cyclopropyl}-3-fluorophenyl)piperazine-1-carboxylate

This compound was prepared by using a procedure that was analogous to that used for the synthesis of example 62. LCMS: (M+H)⁺=418.3.

### Example 130

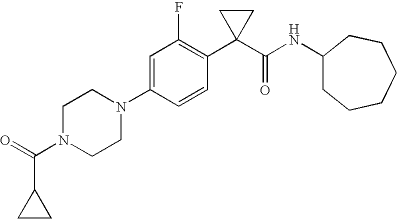

### N-Cycloheptyl-1-{4-[4-(cyclopropylcarbonyl)piperazin-1-yl]-2-fluorophenyl}cyclopropanecarboxamide

This compound was prepared by using a procedure that was analogous to that used for the synthesis of example 62. LCMS: (M+H)⁺=428.3, (M+Na)⁺=450.3.

### Example 131

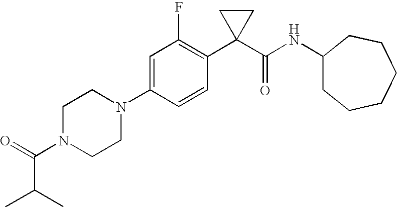

### N-Cycloheptyl-1-[2-fluoro-4-(4-isobutyrylpiperazin-1-yl)phenyl]cyclopropane carboxamide

This compound was prepared by using a procedure that was analogous to that used for the synthesis of example 62. LCMS: (M+H)⁺=430.3, (M+Na)⁺=452.2.

### Example 132

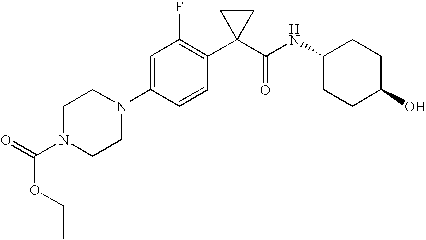

### Ethyl 4-[3-fluoro-4-(1-{[(trans-4-hydroxycyclohexyl)amino]carbonyl}cyclopropyl) phenyl]piperazine-1-carboxylate

This compound was prepared by using a procedure that was analogous to that used for the synthesis of example 62. LCMS: (M+H)⁺=434.3, (M+Na)⁺=456.2.

### Example 133

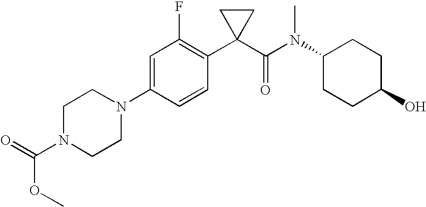

### Methyl 4-[3-fluoro-4-(1-{[(trans-4-hydroxycyclohexyl)(methyl)aminolcarbonyl}cyclopropyl)phenyl]piperazine-1-carboxylate

This compound was prepared by using a procedure that was analogous to that used for the synthesis of example 62. LCMS: (M+H)⁺=434.3, (M+Na)⁺=456.2.

### Example 134

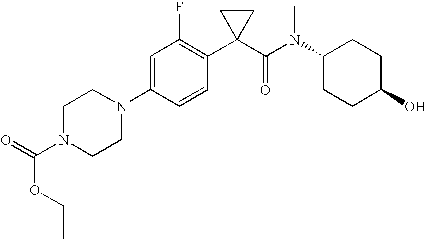

### Ethyl 4-[3-fluoro-4-(1-{[(trans-4-hydroxycyclohexyl)(methyl)amino]carbonyl}cyclopropyl)phenyl]piperazine-1-carboxylate

This compound was prepared by using a procedure that was analogous to that used for the synthesis of example 62. LCMS: (M+H)⁺=448.3, (M+Na)⁺=470.2.

### Example 135

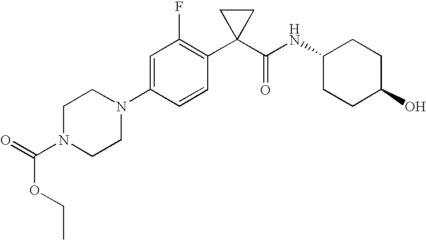

### Ethyl 4-[3-fluoro-4(1-{[(trans-4-hydroxycyclohexyl)amino]carbonyl}cyclopropyl) phenyl]piperazine-1-carboxylate

This compound was prepared by using a procedure that was analogous to that used for the synthesis of example 62. LCMS: (M+H)⁺=434.3, (M+Na)⁺=456.3.

### Example 136

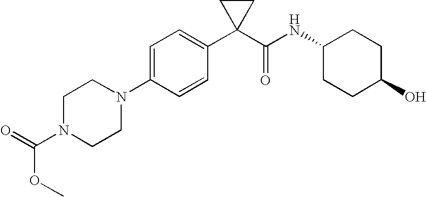

### Methyl 4-[4-(1-{[(trans-4-hydroxycyclohexyl)amino]carbonyl}cyclopropyl)phenyl]piperazine-1-carboxylate

This compound was prepared by using a procedure that was analogous to that used for the synthesis of example 62. LCMS: (M+H)⁺=402.3, (M+Na)⁺=424.3.

### Example 137

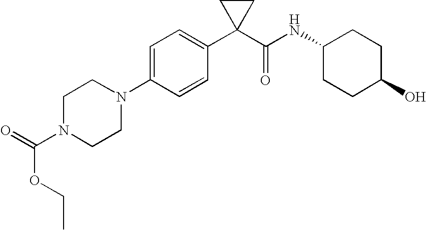

### Ethyl 4-[4-(1-{[(trans-4-hydroxycyclohexyl)amino]carbonyl}cyclopropyl)phenyl]piperazine-1-carboxylate

This compound was prepared by using a procedure that was analogous to that used for the synthesis of example 62. LCMS: (M+H)⁺=416.3, (M+Na)⁺=438.3.

### Example 138

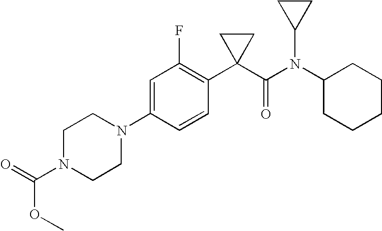

### Methyl 4-[4-(1-{[cyclohexyl(cyclopropyl)amino]carbonyl]cyclopropyl)-3-fluorophenyl]piperazine-1-carboxylate

This compound was prepared by using a procedure that was analogous to that used for the synthesis of example 62. LCMS: (M+H)⁺=444.3.

### Example 139

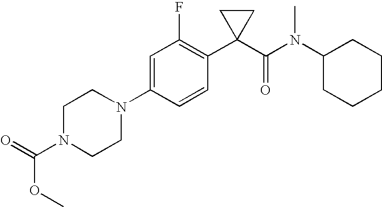

### Methyl 4-[4-(1-{[cyclohexyl(methyl)amino]carbonylcyclopropyl)-3-fluorophenyl]piperazine-1-carboxylate

This compound was prepared by using a procedure that was analogous to that used for the synthesis of example 62. LCMS: (M+H)⁺=418.3.

### Example 140

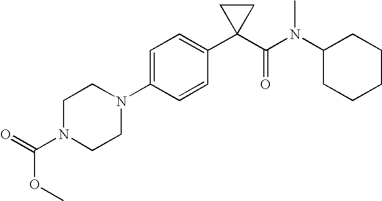

### Methyl 4-[4(1-{[cyclohexyl(methyl)amino]carbonyl}eyclopropyl)phenyl]piperazine-1-carboxylate

This compound was prepared by using a procedure that was analogous to that used for the synthesis of example 62. LCMS: (M+H)⁺=400.3.

### Example 141

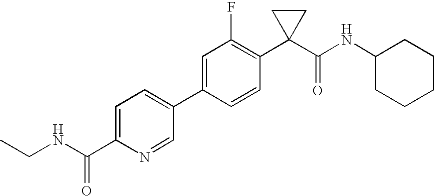

### 5-(4-{1-[(Cyclohexylamino)carbonyl]cyclopropyl}-3-fluorophenyl)-N-ethylpyridine-2-carboxamide

### Step 1. 1-(4-Bromo-2-fluorophenyl)cyclopropanecarbonyl chloride

To 1-(4-bromo-2-fluorophenyl)cyclopropanecarboxylic acid (2.50 g, 0.00965 mol, prepared as an intermediate in the preparation of example 62, step 1) was added thionyl chloride (20 mL, 0.3 mol) at 0° C. and the resulting solution was stirred for 2.5 h at rt. Upon completion, the volatiles were removed in-vacuo and the residue was azeotropically washed with toluene (x3). The crude product was used in the following step without further purification.

### Step 2. 1-(4-Bromo-2-fluorophenyl)-N-cyclohexylcyclopropanecarboxamide

A mixture of 1-(4-bromo-2-fluorophenyl)cyclopropanecarbonyl chloride (55 mg, 0.00020 mol), cyclohexanamine (34 μL, 0.00030 mol), and triethylamine (69 μL, 0.00050 mol) in methylene chloride (0.6 mL, 0.009 mol) was stirred at rt for 4 h. The crude reaction mixture was purified by flash column chromatography to afford 40 mg of the desired product. LCMS: (M+H)⁺=341.1.

### Step 3. N-Cyclohexyl-1-[2-fluoro-4-(4,4,5,5-tetramethyl-1,3,2-dioxaborolan-2-yl)phenyl]cyclopropanecarboxamide

A mixture of 1-(4-bromo-2-fluorophenyl)-N-cyclohexylcyclopropane-carboxamide (40 mg, 0.0001 mol), 4,4,5,5,4′,4′,5′,5′-octamethyl-[2,2′]bi[[1,3,2]dioxaborolanyl] (33 mg, 0.00013 mol), [1,1′-bis(diphenylphosphino) ferrocene]dichloropalladium(II), complex with dichloromethane (1:1) (5 mg, 0.000006 mol), 1,1′-bis(diphenylphosphino)ferrocene (3 mg, 0.000006 mol), and potassium acetate (35 mg, 0.00035 mol) in 1,4-dioxane (0.5 mL, 0.006 mol) was heated at 80° C. for 16 h. After coo the reaction mixture to ambient temperature, the precipitate was filtered off. The filtrate was concentrated in-vacuo and the resulting residue was used in the next step without further purification. LCMS: (M+H)⁺=388.1.

### Step 4. 5-(4-{1-[(Cyclohexylamino)carbonyl]cyclopropyl}-3-fluorophenyl)-N-ethylpyridine-2-carboxamide

A mixture of N-cyclohexyl-1-[2-fluoro-4-(4,4,5,5-tetramethyl-1,3,2-dioxaborolan-2-yl)phenyl]cyclopropanecarboxamide (0.040 g, 0.00010 mol), 5-bromo-N-ethylpyridine-2-carboxamide (0.027 g, 0.00012 mol), [1,1′-bis(diphenylphosphino) ferrocene]-dichloropalladium(II), complex with dichloromethane (1:1) (0.004 g, 0.000005 mol), and potassium carbonate (0.041 g, 0.00030 mol) in N,N-dimethylformamide (0.40 mL, 0.0052 mol) was heated at 120° C. for 16 h. After allowing the reaction mixture to cool to ambient temperature, the crude product was purified by prep-HPLC. LCMS: (M+H)⁺=410.2.

### Example 142

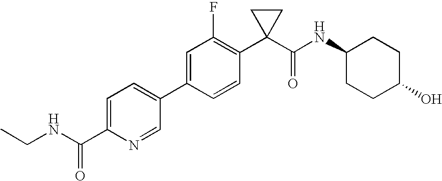

### N-Ethyl-5-[3-fluoro-4-(1-{[(trans-4-hydroxycyclohexyl)amino]carbonyl}cyclopropyl) phenyl]pyridine-2-carboxamide

This compound was prepared by using a procedure that was analogous to that used for the synthesis of example 141, steps 1-4. LCMS: (M+H)⁺=426.3.

### Example 143

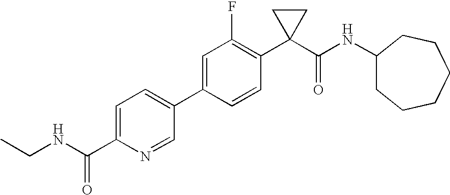

### 5-(4-{1-[(Cycloheptylamino)carbonyl]cyclopropyl}-3-fluorophenyl)-N-ethylpyridine-2-carboxamide

This compound was prepared by using a procedure that was analogous to that used for the synthesis of example 141, steps 1-4. LCMS: (M+H)⁺=424.3.

### Example 144

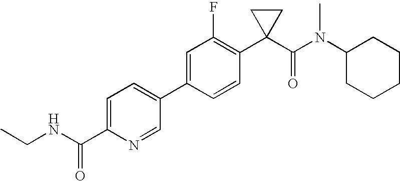

### 5-[4-(1-({[Cyclohexyl(methyl)amino]carbonyl}cyclopropyl)-3-fluorophenyl]-N-ethylpyridine-2-carboxamide

This compound was prepared by using a procedure that was analogous to that used for the synthesis of example 141, steps 1-4. LCMS: (M+H)⁺=424.3.

### Example 145

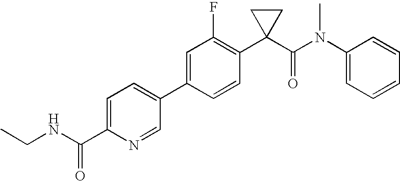

### N-ethyl-5-[3-fluoro-4-(1-{[methyl(phenyl)amino]carbonyl}cyclopropyl)phenyl]pyridine-2-carboxamide

This compound was prepared by using a procedure that was analogous to that used for the synthesis of example 141, steps 1-4. LCMS: (M+H)⁺=418.3.

### Example 146

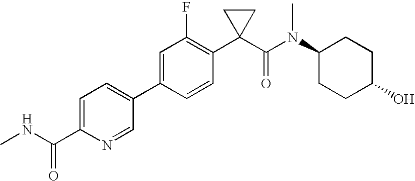

### 5-[3-Fluoro-4-{[(trans-4-hydroxycyclohexyl)(methyl)amino]carbonyl}cyclopropyl)phenyl]-N-methylpyridine-2-carboxamide

This compound was prepared by using a procedure that was analogous to that used for the synthesis of example 141, steps 1-4. LCMS: (M+H)⁺=426.3.

### Example 147

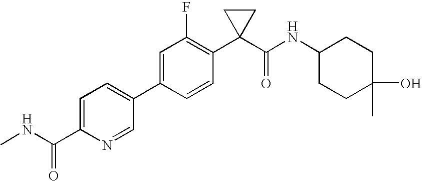

### 5-[3-Fluoro-4-(1-{[(4-hydroxy-4-methylcyclohexyl)amino]carbonyl}cyclopropyl)phenyl]-N-methylpyridine-2-carboxamide

This compound was prepared by using a procedure that was analogous to that used for the synthesis of example 141, steps 1-4. LCMS: (M+H)⁺=426.3.

### Example A

### Enzymatic Assay of 11βHSD1

All in vitro assays were performed with clarified lysates as the source of 11βHSD1 activity. HEK-293 transient transfectants expressing an epitope-tagged version of full-length human 11βHSD1 were harvested by centrifugation. Roughly 2×10⁷ cells were resuspended in 40 mL of lysis buffer (25 mM Tris-HCl, pH 7.5, 0.1M NaCl, 1 mM MgCl₂ and 250 mM sucrose) and lysed in a microfluidizer. Lysates were clarified by centrifugation and the supernatants were aliquoted and frozen.

Inhibition of 11βHSD1 by test compounds was assessed in vitro by a Scintillation Proximity Assay (SPA). Dry test compounds were dissolved at 5 mM in DMSO. These were diluted in DMSO to suitable concentrations for the SPA assay. 0.8 μL of 2-fold serial dilutions of compounds were dotted on 384 well plates in DMSO such that 3 logs of compound concentration were covered. 20 μL of clarified lysate was added to each well. Reactions were initiated by addition of 20 μL of substrate-cofactor mix in assay buffer (25 mM Tris-HCl, pH 7.5, 0.1M NaCl, 1 mM MgCl₂) to final concentrations of 400 μM NADPH, 25 nM ³H-cortisone and 0.007% Triton X-100. Plates were incubated at 37° C. for one hour. Reactions were quenched by addition of 40 μL of anti-mouse coated SPA beads that had been pre-incubated with 10 μM carbenoxolone and a cortisol-specific monoclonal antibody. Quenched plates were incubated for a minimum of 30 minutes at RT prior to reading on a Topcount scintillation counter. Controls with no lysate, inhibited lysate, and with no mAb were run routinely. Roughly 30% of input cortisone is reduced by 11βHSD1 in the uninhibited reaction under these conditions.

Test compounds having an IC₅₀ value less than about 20 μM according to this assay were considered active.

### Example B

Cell-Based Assays for HSD Activity

Peripheral blood mononuclear cells (PBMCs) were isolated from normal human volunteers by Ficoll density centrifugation. Cells were plated at 4×10⁵ cells/well in 200 μL of AIM V (Gibco-BRL) media in 96 well plates. The cells were stimulated overnight with 50 ng/mL recombinant human IL-4 (R&D Systems). The following morning, 200 nM cortisone (Sigma) was added in the presence or absence of various concentrations of compound. The cells were incubated for 48 hours and then supernatants were harvested. Conversion of cortisone to cortisol was determined by a commercially available ELISA (Assay Design).

Test compounds having an IC₅₀ value less than about 20 μM according to this assay were considered active.

### Example C

### Cellular Assay to Evaluate MR Antagonism

Assays for MR antagonism were performed essentially as described (Jausons-Loffreda et al. J Biolumin and Chemilumin, 1994, 9: 217-221). Briefly, HEK293/MSR cells (Invitrogen Corp.) were co-transfected with three plasmids: 1) one designed to express a fusion protein of the GAL4 DNA binding domain and the mineralocorticoid receptor ligand binding domain, 2) one containing the GAL4 upstream activation sequence positioned upstream of a firefly luciferase reporter gene (pFR-LUC, Stratagene, Inc.), and 3) one containing the Renilla luciferase reporter gene cloned downstream of a thymidine kinase promoter (Promega). Transfections were performed using the FuGENE6 reagent (Roche). Transfected cells were ready for use in subsequent assays 24 hours post-transfection.

In order to evaluate a compound's ability to antagonize the MR, test compounds were diluted in cell culture medium (E-MEM, 10% charcoal-stripped FBS, 2 mM L-glutamine) supplemented with 1 nM aldosterone and applied to the transfected cells for 16-18 hours. After the incubation of the cells with the test compound and aldosterone, the activity of firefly luciferase (indicative of MR agonism by aldosterone) and Renilla luciferase (normalization control) are determined using the Dual-Glo Luciferae Assay System (Promega). Antagonism of the mineralocorticoid receptor was determined by monitoring the ability of a test compound to attenuate the aldosterone-induced firefly luciferase activity.

Compounds having an IC₅₀ of 100 μM or less were considered active.

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

1. A compound of Formula I: 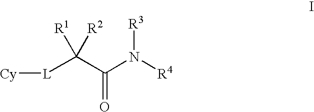
or pharmaceutically acceptable salt or prodrug thereof, wherein:
Cy is aryl, heteroaryl, cycloalkyl or heterocycloalkyl, each optionally substituted by 1, 2, 3, 4 or 5-W—X—Y-Z;
L is SO₂, (CR⁶R⁷)ₙO(CR⁶R⁷)ₚ or (CR⁶R⁷)ₙS(CR⁶R⁷)ₚ;
R¹ and R² together with the C atom to which they are attached form a 3-, 4-, 5-, 6- or 7-membered cycloalkyl group or a 3-, 4-, 5-, 6- or 7-membered heterocycloalkyl group, each optionally substituted by 1, 2 or 3 R⁵;
R³ is H, C₁₋₆alkyl, cycloalkyl, heterocycloalkyl, arylalkyl, cycloalkylalkyl, heteroarylalkyl, or heterocycloalkylalkyl;
R⁴ is C₁₋₆ alkyl, cycloalkyl, heterocycloalkyl, arylalkyl, cycloalkylalkyl, heteroarylalkyl, heterocycloalkylalkyl, each optionally substituted by 1, 2 or 3-W′—X′—Y′-Z′;
wherein when R³ is C₁₋₆ alkyl, R⁴ is other than C₁₋₆ alkyl;
R5 is halo, C₁₋₄ alkyl, C₁₋₄ haloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, or NR^{c}C(O)OR^{a};
R⁶ and R⁷ are each, independently, H, halo, C₁₋₄ alkyl, C₁₋₄ haloalkyl, aryl, cycloalkyl, heteroaryl, heterocycloalkyl, CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}, NR^{c′}R^{d′}, NR^{c′}C(O)R^{d′}, NR^{c′}C(O)OR^{a′}, S(O)R^{b′}, S(O)NR^{c′}R^{d′}, S(O)₂R^{b′}, or S(O)₂NR^{c′}R^{d′};
W, W′ and W″ are each, independently, absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, or NR^{e}CONR^{f}, wherein said C₁₋₆ alkylenyl, C₂₋₆ alkenylenyl, C₂₋₆ alkynylenyl are each optionally substituted by 1, 2 or 3 halo, OH, C₁₋₄ alkoxy, C₁₋₄ haloalkoxy, amino, C₁₋₄ alkylamino or C₂₋₈ dialkylamino;
X, X′ and X″ are each, independently, absent, C₁₋₆ alkylenyl, C₂₋₆ alkenylenyl, C₂₋₆ alkynylenyl, aryl, cycloalkyl, heteroaryl or heterocycloalkyl, wherein said C₁₋₆ alkylenyl, C₂₋₆ alkenylenyl, C₂₋₆ alkynylenyl, cycloalkyl, heteroaryl or heterocycloalkyl is optionally substituted by one or more halo, CN, NO₂, OH, C₁₋₄ alkoxy, C₁₋₄ haloalkoxy, amino, C₁₋₄ alkylamino or C₂₋₈ dialkylamino;
Y, Y′ and Y″ are each, independently, absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, or NR^{e}CONR^{f}, wherein said C₁₋₆ alkylenyl, C₂₋₆ alkenylenyl, C₂₋₆ alkynylenyl are each optionally substituted by 1, 2 or 3 halo, OH, C₁₋₄ alkoxy, C₁₋₄ haloalkoxy, amino, C₁₋₄ alkylamino or C₂₋₈ dialkylamino;
Z, Z′ and Z″ are each, independently, H, halo, CN, NO₂, OH, C₁₋₄ alkoxy, C₁₋₄ haloalkoxy, amino, C₁₋₄ alkylamino or C₂₋₈ dialkylamino, C₁₋₆ alkyl, C₂₋₆ alkenyl, C₂₋₆ alkynyl, aryl, cycloalkyl, heteroaryl or heterocycloalkyl, wherein said C₁₋₆ alkyl, C₂₋₆ alkenyl, C₂₋₆ alkynyl, aryl, cycloalkyl, heteroaryl or heterocycloalkyl is optionally substituted by 1, 2 or 3 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, S(O)R^{b}, S(O)NR^{c}R^{d}, S(O)₂R^{b}, or S(O)₂NR^{c}R^{d};
wherein two —W—X—Y-Z attached to the same atom, together with the atom to which they are attached, optionally form a 3-20 membered cycloalkyl or heterocycloalkyl group each optionally substituted by 1, 2 or 3-W″—X″—Y″-Z″;
or wherein two —W—X—Y-Z together with the carbon atom to which they are both attached optionally form a carbonyl;
or wherein two —W—X—Y-Z together with two adjacent atoms to which they are attached optionally form a 3-20 membered fused cycloalkyl group or 3-20 membered fused heterocycloalkyl group, each optionally substituted by 1, 2 or 3-W″—X″—Y″-Z″;
wherein two —W′—X′—Y′-Z′ together with the atom to which they are both attached optionally form a 3-20 membered cycloalkyl group or 3-20 membered heterocycloalkyl group, each optionally substituted by 1, 2 or 3-W″—X″—Y″-Z″;
or wherein two —W′—X′—Y′-Z′ together with the carbon atom to which they are both attached optionally form a carbonyl;
or wherein two —W′—X′—Y′-Z′ together with two adjacent atoms to which they are attached optionally form a 3-20 membered fused cycloalkyl group or 3-20 membered fused heterocycloalkyl group, each optionally substituted by 1, 2 or 3-W″—X″—Y″-Z″;
or wherein two —W′—X′—Y′-Z′ together with two adjacent atoms to which they are attached optionally form a 5- or 6-membered fused aryl or 5- or 6-membered fused heteroaryl group, each optionally substituted by 1, 2 or 3-W″—X″—Y″-Z″;
wherein —W—X—Y-Z is other than H;
wherein —W′—X′—Y′-Z′ is other than H;
wherein —W″—X″—Y″-Z″ is other than H;
R^{a} and R^{b} are each, independently, H, C₁₋₆ alkyl, C₁₋₆ haloalkyl, C₂₋₆ alkenyl, C₂₋₆ alkynyl, aryl, cycloalkyl, heteroaryl or heterocycloalkyl;
R^{b} and R^{b′} are each, independently, H, C₁₋₆ alkyl, C₁₋₆ haloalkyl, C₂₋₆ alkenyl, C₂₋₆ alkynyl, aryl, cycloalkyl, heteroaryl or heterocycloalkyl;
R^{c} and R^{d} are each, independently, H, C₁₋₆ alkyl, C₁₋₆ haloalkyl, C₂₋₆ alkenyl, C₂₋₆ alkynyl, aryl, cycloalkyl, arylalkyl, or cycloalkylalkyl;
or R^{c} and R^{d} together with the N atom to which they are attached form a 4-, 5-, 6- or 7-membered heterocycloalkyl group;
R^{c′} and R^{d′} are each, independently, H, C₁₋₆ alkyl, C₁₋₆ haloalkyl, C₂₋₆ alkenyl, C₂₋₆ alkynyl, aryl, cycloalkyl, arylalkyl, or cycloalkylalkyl;
or R^{c′} and R^{d′} together with the N atom to which they are attached form a 4-, 5-, 6- or 7-membered heterocycloalkyl group;
R^{e} and R^{f} are each, independently, H, C₁₋₆ alkyl, C₁₋₆ haloalkyl, C₂₋₆ alkenyl, C₂₋₆ alkynyl, aryl, cycloalkyl, arylalkyl, or cycloalkylalkyl;
or R^{e} and R^{f} together with the N atom to which they are attached form a 4-, 5-, 6- or 7-membered heterocycloalkyl group;
n is 0, 1, 2 or 3; and
p is 0, 1, 2 or 3;
with the proviso that when L is SCH₂ and R³ is H, then R⁴ is other than 4-benzyloxycarbonyl-6-oxo-1,3,4,7,8,12b-hexahydro-2H-benzo[c]pyrido[1,2-a]azepin-7-yl.

2. The compound of claim 1 wherein Cy is aryl or heteroaryl, each optionally substituted by 1, 2, 3, 4 or 5-W—X—Y-Z.

3. The compound of claim 1 wherein Cy is aryl optionally substituted by 1, 2, 3, 4 or 5-W—X—Y-Z.

4. The compound of claim 1 wherein Cy is phenyl optionally substituted by 1, 2, 3, 4 or 5-W—X—Y-Z.

5. The compound of claim 1 wherein Cy is phenyl optionally substituted by 1, 2, 3, 4 or 5 halo.

6. The compound of claim 1 wherein L is OCH₂.

7. The compound of claim 1 wherein L is S or SCH₂.

8. The compound of claim 1 wherein R¹ and R² together with the C atom to which they are attached form cyclopropyl optionally substituted by 1, 2 or 3 R⁵.

9. The compound of claim 1 wherein R¹ and R² together with the C atom to which they are attached form cyclopropyl.

10. The compound of claim1wherein R³ is H, C₁₋₆ alkyl, cyclopropyl, cyclobutyl, cyclopentyl, cyclohexyl, cycloheptyl, indanyl, 1,2,3,4-tetrahydro-naphthyl, bicyclo[2.2.1]heptanyl, piperidinyl, piperazinyl, pyrrolidinyl, tetrahydrofuranyl, dihydro-furan-2-on-yl, cyclopropylethyl, cyclopropylpropyl, cyclohexylethyl, cyclohexylpropyl, cyclohexylbutyl, phenylpropyl, phenylbutyl, 2,3-dihydro-benzo[1,4]dioxinylmethyl, 1H-indolylethyl, 1H-indolylpropyl or 1H-indolylbutyl, each optionally substituted by 1, 2 or 3-W′—X′—Y′-Z′.

11. The compound of claim 1 wherein R³ is H, cyclopropyl, cyclopentyl, or cyclohexyl.

12. The compound of claim 1 wherein R³ is H or cyclopropyl.

13. The compound of claim 1 wherein R⁴ is C₁₋₆ alkyl, cyclopropyl, cyclobutyl, cyclopentyl, cyclohexyl, cycloheptyl, indanyl, adamantyl, 1,2,3,4-tetrahydro-naphthyl, bicyclo[2.2.1]heptanyl (norbornyl), piperidinyl, piperazinyl, pyrrolidinyl, tetrahydrofuranyl, dihydro-furan-2-on-yl, tetrahydropyranyl, cyclopropylethyl, cyclopropylpropyl, cyclohexylmethyl, cyclohexylethyl, cyclohexylpropyl, cyclohexylbutyl, phenylethyl, phenylpropyl, phenylbutyl, 2,3-dihydro-benzo[1,4]dioxinylmethyl, pyridinylmethyl, pyridinylethyl, 1H-indolylethyl, 1H-indolylpropyl or 1H-indolylbutyl, each optionally substituted by 1, 2 or 3-W′—X′—Y′-Z′.

14. The compound of claim 1 wherein —W—X—Y-Z is halo, C₁₋₄ alkyl, C₁₋₄ haloalkyl, OH. C₁₋₄ alkoxy, C₁₋₄ haloalkoxy, (alkoxy)-CO-cycloalkyl, (alkoxy)-CO-heterocycloalkyl, hydroxyalkyl, alkoxyalkyl, cycloalkyl, heterocycloalkyl, aryl, heteroaryl, arylalkyl or heteroarylalkyl.

15. The compound of claim 1 wherein —W—X—Y-Z is halo, heteroaryl, or heterocycloalkyl.

16. The compound of claim 1 wherein —W—X—Y-Z is halo.

17. The compound of claim 1 wherein —W′—X′—Y′-Z′ is halo, C₁₄ alkyl, C₁₋₄ haloalkyl, OH, C₁₋₄ alkoxy, C₁₋₄ haloalkoxy, hydroxyalkyl, alkoxyalkyl, —COO-alkyl, aryl, heteroaryl, aryloxy, heteroaryloxy, arylalkyloxy, heteroarylalkyloxy, optionally substituted arylsulfonyl, optionally substituted heteroarylsulfonyl, aryl substituted by halo, heteroaryl substituted by halo.

18. A compound of Formula I: 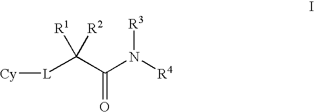
or pharmaceutically acceptable salt or prodrug thereof, wherein:
Cy is phenyl or heteroaryl, each optionally substituted by 1, 2, 3, 4 or 5 R^{1a};
L is absent or (CR⁶R⁷)ₘ;
R¹ and R² together with the carbon atom to which they are attached form cyclopropyl or cyclobutyl;
R³ is H, C₁₋₆ alkyl, cycloalkyl, heterocycloalkyl, or cycloalkylalkyl;
R⁴ is cyclopropyl, (CR^{4a}R^{4b})ₙCy², (CR^{4a}R^{4b})ₜCy³, (CHR^{4c})Cy³, (CR^{4a}R^{4b})ₜ₁Cy⁴, (CR^{4a}R^{4b})ₜCH₂OH, (CR^{4a}R^{4b})ₜ—O-phenyl, —CR^{6a}R^{7a}R^{8a}, or (CH₂)ₜCy⁵, wherein said cyclopropyl is optionally substituted by 1, 2 or 3 halo, C₁₋₃ alkyl, C₁₋₃ haloalkyl, phenyl, benzyl, C(O)OR^{10a} or OR^{10a};
R⁶ and R⁷ are each, independently, H, halo, C₁₋₄ alkyl, C₁₋₄ haloalkyl, aryl, cycloalkyl, heteroaryl, heterocycloalkyl, CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}, NR^{c′}R^{d′}, NR^{c′}C(O)R^{d′}, NR^{c′}C(O)OR^{a′}, S(O)R^{b′}, S(O)NR^{c′}R^{d′}, S(O)₂R^{b′}, or S(O)₂NR^{c′}R^{d′};
R^{1a} and R^{1b} are each, independently, halo, CN, NO₂, OH, OR^{a}, SR^{a}, C(O)R^{b}, C(O)NR^{c}R^{d}, C(O)OR^{a}, OC(O)R^{b}, OC(O)NR^{c}R^{d}, NRCR^{d}, NR^{c}C(O)R^{d}, NR^{c}C(O)OR^{a}, S(O)R^{b}, S(O)NR^{c}R^{d}, S(O)₂R^{b}, S(O)₂NR^{c}R^{d}, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, aryl, arylsulfonyl, cycloalkyl, heteroaryl or heterocycloalkyl, wherein said C₁₋₆ alkyl, C₂₋₆ alkenyl, C₂₋₆ alkynyl, arylsulfonyl, aryl, cycloalkyl, heteroaryl or heterocycloalkyl is optionally substituted by 1, 2 or 3 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, S(O)R^{b}, S(O)NR^{c}R^{d}, S(O)₂R^{b}, or S(O)₂NR^{c}R^{d};
R^{4a} and R^{4b} are each, independently, H, halo, OH, CN, C₁₋₄alkyl, C₁₋₄ alkoxy, wherein said C₁₋₄ alkyl or C₁₋₄ alkoxy is optionally substituted with one or more halo, CN, NO₂, OH, C₁₋₄ alkoxy, C₁₋₄ haloalkoxy, amino, C₁₋₄ alkylamino or C₂₋₈ dialkylamino;
R^{4c} is OH, CN, C₁₋₄ alkyl, C₁₋₄ alkoxy, wherein said C₁₋₄ alkyl or C₁₋₄ alkoxy is optionally substituted with one or more halo, CN, NO₂, OH, C₁₋₄ alkoxy, C₁₋₄ haloalkoxy, amino, C₁₋₄ alkylamino or C₂₋₈ dialkylamino;
R^{5a} and R^{5b} are each, independently, H, halo, OH, CN, C₁₋₄ alkyl, C₁₋₄ alkoxy, wherein said C₁₋₄ alkyl or C₁₋₄ alkoxy is optionally substituted with one or more halo, CN, NO₂, OH, C₁₋₄ alkoxy, C₁₋₄ haloalkoxy, amino, C₁₋₄ alkylamino or C₂₋₈ dialkylamino;
R^{6a} is H or methyl;
R^{7a} is methyl or CH₂OH;
R^{8a} is C₂₋₆ alkyl or —(CR^{5a}R^{5b})ₚR^{9a}, wherein said C₂₋₆ alkyl is optionally substituted with one or more halo, CN, NO₂, OH, C₁₋₄ alkoxy or C₁₋₄ haloalkoxy;
R^{9a} is halo, CN, NO₂, OH, C₁₋₄ alkoxy, C₁₋₄ haloalkoxy, amino, C₁₋₄ alkylamino, C₂₋₈ dialkylamino, OR^{10b}, SR^{10b}, C(O)R^{10b}, C(O)NR^{10b}R^{11b}, C(O)OR^{10b}, OC(O)R^{10b}, OC(O)NR^{10b}R^{11b}, NR^{10b}R^{11b}, NR^{10b}C(O)R^{11b}, NR^{10b}C(O)OR^{11b}, S(O)R^{10b}, S(O)NR^{10b}R^{11b}, S(O)₂R^{11b}, S(O)₂NR^{10b}R^{11b}, cycloalkyl, aryl, heteroaryl, wherein said cycloalkyl, aryl or heteroaryl is optionally substituted by one or more halo, C₁₋₄ alkyl, C₁₋₄ haloalkyl, CN, NO₂, OH, C₁₋₄ alkoxy, C₁₋₄ haloalkoxy, amino, C₁₋₄ alkylamino or C₂₋₈ dialkylamino;
R^{10a} is H, C₁₋₆ alkyl, C₁₋₆ haloalkyl, C₂₋₆ alkenyl, C₂₋₆ alkynyl, aryl, cycloalkyl, heteroaryl or heterocycloalkyl;
R^{10b} and R^{11b} are each, independently, H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, cycloalkylalkyl, heteroarylalkyl or heterocycloalkylalkyl;
or R^{10b} and R^{11b} together with the N atom to which they are attached form a 4-, 5-, 6- or 7-membered heterocycloalkyl group;
Cy ² is: 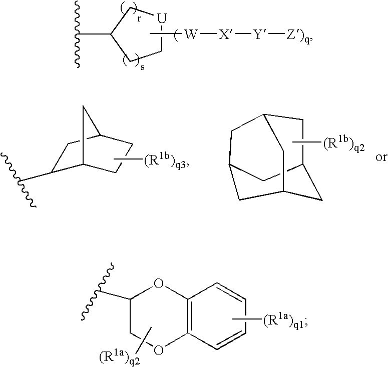
Cy³ isphenyl optionally substituted by 1, 2, 3, 4 or 5 R^{1b};
Cy⁴ is pyridinyl optionally substituted by 1, 2, 3, 4 or 5 R^{1b};
Cy⁵ is phenyl optionally substituted by 1, 2, 3, 4 or 5 halo or OH;
U is CH₂, NH, or O;
W′ and W″ are each, independently, absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, or NR^{e}CONR^{f}, wherein said C₁₋₆ alkylenyl, C₂₋₆ alkenylenyl, C₂₋₆ alkynylenyl are each optionally substituted by 1, 2 or 3 halo, OH, C₁₋₄ alkoxy, C₁₋₄ haloalkoxy, amino, C₁₋₄ alkylamino or C₂₋₈ dialkylamino;
X′ and X″ are each, independently, absent, C₁₋₆ alkylenyl, C₂₋₆ alkenylenyl, C₂₋₆ alkynylenyl, aryl, cycloalkyl, heteroaryl or heterocycloalkyl, wherein said C₁₋₆ alkylenyl, C₂₋₆ alkenylenyl, C₂₋₆ alkynylenyl, cycloalkyl, heteroaryl or heterocycloalkyl is optionally substituted by one or more halo, CN, NO₂, OH, C₁₋₄ alkoxy, C₁₋₄ haloalkoxy, amino, C₁₋₄ alkylamino or C₂₋₈ dialkylamino;
Y′ and Y″ are each, independently, absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, or NR^{e}CONR^{f}, wherein said C₁₋₆ alkylenyl, C₂₋₆ alkenylenyl, C₂₋₆ alkynylenyl are each optionally substituted by 1, 2 or 3 halo, OH, C₁₋₄ alkoxy, C₁₋₄ haloalkoxy, amino, C₁₋₄ alkylamino or C₂₋₈ dialkylamino;
Z′ and Z″ are each, independently, H, halo, CN, NO₂, OH, C₁₋₄ alkoxy, C₁₋₄ haloalkoxy, amino, C₁₋₄ alkylamino or C₂₋₈ dialkylamino, C₁₋₆ alkyl, C₂₋₆ alkenyl, C₂₋₆ alkynyl, aryl, cycloalkyl, heteroaryl or heterocycloalkyl, wherein said C₁₋₆ alkyl, C₂₋₆ alkenyl, C₂₋₆ alkynyl, aryl, cycloalkyl, heteroaryl or heterocycloalkyl is optionally substituted by 1, 2 or 3 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, S(O)R^{b}, S(O)NR^{c}R^{d}, S(O)₂R^{b}, or S(O)₂NR^{c}R^{d};
wherein two —W′—X′—Y′-Z′ together with the atom to which they are both attached optionally form a 3-20 membered cycloalkyl group or 3-20 membered heterocycloalkyl group, each optionally substituted by 1, 2 or 3-W″—X″—Y″-Z″;
or wherein two —W′—X′—Y′-Z′ together with the carbon atom to which they are both attached optionally form a carbonyl;
wherein two —W′—X′—Y′-Z′ together with two adjacent atoms to which they are attached optionally form a 3-20 membered fused cycloalkyl group or 3-20 membered fused heterocycloalkyl group, each optionally substituted by 1, 2 or 3-W″—X″—Y″-Z″;
or wherein two —W′—X′—Y′-Z′ together with two adjacent atoms to which they are attached optionally form a 5- or 6-membered fused aryl or 5- or 6-membered fused heteroaryl group, each optionally substituted by 1, 2 or 3-W″—X″—Y″-Z″;
wherein —W′—X′—Y′-Z′ is other than H;
wherein —W″—X″—Y″-Z″ is other than H;
R^{a} and R^{a′} are each, independently, H, C₁₋₆ alkyl, C₁₋₆ haloalkyl, C₂₋₆ alkenyl, C₂₋₆ alkynyl, aryl, cycloalkyl, heteroaryl or heterocycloalkyl;
R^{b} and R^{b′} are each, independently, H, C₁₋₆ alkyl, C₁₋₆ haloalkyl, C₂₋₆ alkenyl, C₂₋₆ alkynyl, aryl, cycloalkyl, heteroaryl or heterocycloalkyl;
R^{c} and R^{d} are each, independently, H, C₁₋₆ alkyl, C₁₋₆ haloalkyl, C₂₋₆ alkenyl, C₂₋₆ alkynyl, aryl, cycloalkyl, arylalkyl, or cycloalkylalkyl;
or R^{e} and R^{d} together with the N atom to which they are attached form a 4-, 5-, 6- or 7-membered heterocycloalkyl group;
R^{c′} and R^{d′} are each, independently, H, C₁₋₆ alkyl, C₁₋₆ haloalkyl, C₂₋₆ alkenyl, C₂₋₆ alkynyl, aryl, cycloalkyl, arylalkyl, or cycloalkylalkyl;
or R^{c′} and R^{d′} together with the N atom to which they are attached form a 4-, 5-, 6- or 7-membered heterocycloalkyl group;
R^{e} and R^{f} are each, independently, H, C₁₋₆ alkyl, C₁₋₆ haloalkyl, C₂₋₆ alkenyl, C₂₋₆ alkynyl, aryl, cycloalkyl, arylalkyl, or cycloalkylalkyl;
or R^{e} and R^{f} together with the N atom to which they are attached form a 4-, 5-, 6- or 7-membered heterocycloalkyl group;
m is 1, 2, 3 or 4;
n is 0, 1, 2, or 3;
t1 is 1, 2, or 3;
t is 2 or 3;
s is 1 or 2;
p is 1, 2, 3, 4 or 5;
q1 is 0, 1, 2, 3 or 4;
q2 is 0, 1, 2 or 3;
q3 is 1, 2 or 3;
q is 0, 1, 2, 3, 4 or 5; and
r is 1 or 2; wherein:
a) when L is absent and R⁴ is (CR^{4a}R^{4b})ₜCy³, then at least one of R^{4a} and R^{4b} is other than H;
b) when L is absent, R⁴ is (CR^{4a}R^{4b})ₙCy² and n is 0, then Cy² is other than unsubstituted cyclopentyl, 2-methylcyclohexyl, 4-[(7-chlorquinolin-4-yl)amino]cyclohexyl, 3-(9-chloro-3-methyl-4-oxoisoxazolo[4,3-c]quinolin-5(4H)-yl)cyclohexyl, 1-[3-(2-methoxyphenoxy)benzyl]-piperidin-4-yl, 1-[3-(2-methoxyphenoxy)benzyl]-pyrrolidin-3-yl, or 1,7,7-trimethylbicyclo[2.2.1]hept-2-yl;
c) when L is absent, R⁴ is (CR^{4a}R^{4b})ₙCy² and n is 1, then Cy² is other than 1,3,4,6,7,11b-hexahydro-9-methoxy-2H-benzo[a]quinolizin-2-yl;
d) when L is absent, R⁴ is (CR^{4a}R^{4b})ₙCy² and Cy² is unsubstituted admantyl, then Cy is other than phenyl;
e) when L is absent, R⁴ is (CHR^{4c})Cy³ and R^{4c} is methyl, then Cy is other than unsubstituted phenyl; and
f) when L is absent, R⁴ is (CR^{4a}R^{4b})ₜ₁Cy⁴ and t1 is 1, then then Cy is other than unsubstituted phenyl.

19. The compound of claim 18 wherein L is absent.

20. The compound of claim 18 wherein Cy is phenyl optionally substituted by 1, 2, 3, 4 or 5 R^{1a}.

21. The compound of claim 18 wherein R¹ and R² together with the carbon atom to which they are attached form cyclopropyl.

22. The compound of claim 18 wherein R^{1a} is halo, C₁₋₄ alkoxy, heterocycloalkyl, or heteroaryl, wherein said heterocycloalkyl or heteroaryl is optionally substituted by 1, 2 or 3 C(O)OR^{a}, CONR^{c}R^{d}, or COR^{b}.

23. The compound of claim 18 wherein R^{1a} is halo or C₁₋₄ alkoxy

24. The compound of claim 18 wherein R³ is H or C₁₋₆ alkyl

25. The compound of claim 18 wherein R⁴ is (CR^{4a}R^{4b})ₙCy².

26. The compound of claim 18 wherein R⁴ is

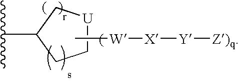

27. The compound of claim 26 wherein U is CH₂.

28. The compound of claim 26 wherein U is NH or O.

29. The compound of claim 18 wherein R⁴ is cyclohexyl.

30. The compound of claim 18 wherein:
Cy is phenyl optionally substituted by 1, 2, 3, 4 or 5 R^{1a};
L is absent or (CR⁶R ⁷)ₘ;
R¹ and R² together with the carbon atom to which they are attached form cyclopropyl;
R³ is H, cyclopropyl, or C₁₋₆ alkyl;
R⁴ is cyclopropyl, (CR^{4a}R^{4b})ₙCy², (CR^{4a}R^{4b})ₜCy³, or —CR^{6a}R^{7a}R^{8a}, wherein said cyclopropyl is optionally substituted by 1, 2 or 3 halo, C₁₋₃ alkyl, C₁₋₃ haloalkyl, phenyl, benzyl, C(O)OR^{10a} or OR^{10a};
R⁶ and R⁷ are each, independently, H, halo, C₁₋₄ alkyl, C₁₋₄ haloalkyl, aryl, cycloalkyl, heteroaryl, heterocycloalkyl, CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}, NR^{c′}R^{d′}, NR^{c′}C(O)R^{d′}, NR^{c′}C(O)OR^{a′}, S(O)R^{b′}, S(O)NR^{c′}R^{d′}, S(O)₂R^{b′}, or S(O)₂NR^{c′}R^{d′};
R^{1a} and R^{1b} are each, independently, halo, CN, NO₂, OH, OR^{a}, SR^{a}, C(O)R^{b}, C(O)NR^{c}R^{d}, C(O)R^{a}, OC(O)R^{b}, OC(O)NR^{c}R^{d}, NR^{c}R^{d}, NR^{c}C(O)R^{d}, NR^{c}C(O)OR^{a}, S(O)R^{b}, S(O)NR^{c}R^{d}, S(O)₂R^{b}, S(O)₂NR^{c}R^{d}, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, aryl, cycloalkyl, heteroaryl or heterocycloalkyl, wherein said C₁₋₆ alkyl, C₂₋₆ alkenyl, C₂₋₆ alkynyl, aryl, cycloalkyl, heteroaryl or heterocycloalkyl is optionally substituted by 1, 2 or 3 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, S(O)R^{b}, S(O)NR^{c}R^{d}, S(O)₂R^{b}, or S(O)₂NR^{c}R^{d};
R^{4a} and R^{4b} are each, independently, H, halo, OH, CN, C₁₋₄alkyl, C₁₋₄ alkoxy, wherein said C₁₋₄ alkyl or C₁₋₄ alkoxy is optionally substituted with one or more halo, CN, NO₂, OH, C₁₋₄ alkoxy, C₁₋₄ haloalkoxy, amino, C₁₋₄ alkylamino or C₂₋₈ dialkylamino;
R^{5a} and R^{5b} are each, independently, H, halo, OH, CN, C₁₋₄ alkyl, C₁₋₄ alkoxy, wherein said C₁₋₄ alkyl or C₁₋₄ alkoxy is optionally substituted with one or more halo, CN, NO₂, OH, C₁₋₄ alkoxy, C₁₋₄ haloalkoxy, amino, C₁₋₄ alkylamino or C₂₋₈ dialkylamino;
R^{6a} is H or methyl;
R^{7a} is methyl or CH₂OH;
R^{8a} is C₂₋₆ alkyl or —(CR^{5a}R^{5b})ₚR^{9a}, wherein said C₂₋₆ alkyl is optionally substituted with one or more halo, CN, NO₂, OH, C₁₋₄ alkoxy or C₁₋₄ haloalkoxy;
R^{9a} is halo, CN, NO₂, OH, C₁₋₄ alkoxy, C₁₋₄ haloalkoxy, amino, C₁₋₄ alkylamino, C₂₋₈ dialkylamino, OR^{10b}, SR^{10b}, C(O)R^{10b}, C(O)NR^{10b}R^{11b}, C(O)OR^{11b}, OC(O)R^{10b}, OC(O)NR^{10b}R^{11b}, NR^{10b}R^{11b}, NR^{10b}C(O)R^{11b}, NR^{10b}C(O)OR^{11b}, S(O)R^{10b}, S(O)NR^{10b}R^{11b}, S(O)₂R^{11b}, S(O)₂NR^{10b}R^{11b}, cycloalkyl, aryl, heteroaryl, wherein said cycloalkyl, aryl or heteroaryl is optionally substituted by one or more halo, C₁₋₄ alkyl, C₁₋₄ haloalkyl, CN, NO₂, OH, C₁₋₄ alkoxy, C₁₋₄ haloalkoxy, amino, C₁₋₄ alkylamino or C₂₋₈ dialkylamino;
R^{10a} is H, C₁₋₆ alkyl, C₁₋₆ haloalkyl, C₂₋₆ alkenyl, C₂₋₆ alkynyl, aryl, cycloalkyl, heteroaryl or heterocycloalkyl;
R^{10b} and R^{11b} are each, independently, H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, cycloalkylalkyl, heteroarylalkyl or heterocycloalkylalkyl;
or R^{10b} and R^{11b} together with the N atom to which they are attached form a 4-, 5-, 6- or 7-membered heterocycloalkyl group;
Cy² is: 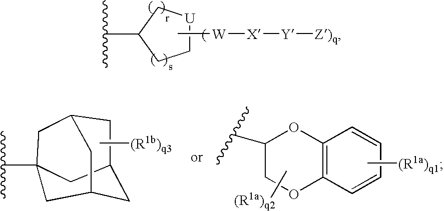
Cy³ is phenyl optionally substituted by 1, 2, 3, 4 or 5 R^{1b};
U is CH₂, NH, or O;
W′—X′—Y′-Z′ is halo, CN, NO₂, OH, C₁₋₄ alkoxy, C₁₋₄ haloalkoxy, amino, C₁₋₄ alkylamino, C₂₋₈ dialkylamino, C₂₋₆ alkyl, C₂₋₆ alkenyl, C₂₋₆ alkynyl, aryl, cycloalkyl, wherein said C₂₋₆ alkyl, C₂₋₆ alkenyl, C₂₋₆ alkynyl, aryl, or cycloalkyl is optionally substituted by 1, 2 or 3 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, S(O)R^{b}, S(O)NR^{c}R^{d}, S(O)₂R^{b}, or S(O)₂NR^{c}R^{d};
W″ is absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, or NR^{e}CONR^{f}, wherein said C₁₋₆ alkylenyl, C₂₋₆ alkenylenyl, C₂₋₆ alkynylenyl are each optionally substituted by 1, 2 or 3 halo, OH, C₁₋₄ alkoxy, C₁₋₄ haloalkoxy, amino, C₁₋₄ alkylamino or C₂₋₈ dialkylamino;
X″ is absent, C₁₋₆ alkylenyl, C₂₋₆ alkenylenyl, C₂₋₆ alkynylenyl, aryl, cycloalkyl, heteroaryl or heterocycloalkyl, wherein said C₁₋₆ alkylenyl, C₂₋₆ alkenylenyl, C₂₋₆ alkynylenyl, cycloalkyl, heteroaryl or heterocycloalkyl is optionally substituted by one or more halo, CN, NO₂, OH, C₁₋₄ alkoxy, C₁₋₄ haloalkoxy, amino, C₁₋₄ alkylamino or C₂₋₈ dialkylamino;
Y″ is absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, or NR^{e}CONR^{f}, wherein said C₁₋₆ alkylenyl, C₂₋₆ alkenylenyl, C₂₋₆ alkynylenyl are each optionally substituted by 1, 2 or 3 halo, OH, C₁₋₄ alkoxy, C₁₋₄ haloalkoxy, amino, C₁₋₄ alkylamino or C₂₋₈ dialkylamino;
Z″ is H, halo, CN, NO₂, OH, C₁₋₄ alkoxy, C₁₋₄ haloalkoxy, amino, C₁₋₄ alkylamino or C₂₋₈ dialkylamino, C₁₋₆ alkyl, C₂₋₆ alkenyl, C₂₋₆ alkynyl, aryl, cycloalkyl, heteroaryl or heterocycloalkyl, wherein said C₁₋₆ alkyl, C₂₋₆ alkenyl, C₂₋₆ alkynyl, aryl, cycloalkyl, heteroaryl or heterocycloalkyl is optionally substituted by 1, 2 or 3 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, S(O)R^{b}, S(O)NR^{c}R^{d}, S(O)₂R^{b}, or S(O)₂NR^{c}R^{d};
wherein two —W′—X′—Y′-Z′ together with the atom to which they are both attached optionally form a 3-20 membered cycloalkyl group or 3-20 membered heterocycloalkyl group, each optionally substituted by 1, 2 or 3-W″—X″—Y″-Z″;
or wherein two —W′—X′—Y′-Z′ together with the carbon atom to which they are both attached optionally form a carbonyl;
or wherein two —W′—X′—Y′-Z′ together with two adjacent atoms to which they are attached optionally form a 5- or 6-membered fused aryl optionally substituted by 1, 2 or 3-W″—X″—Y″-Z″;
wherein —W″—X″—Y″-Z″ is other than H;
R^{a} and R^{a′} are each, independently, H, C₁₋₆ alkyl, C₁₋₆ haloalkyl, C₂₋₆ alkenyl, C₂₋₆ alkynyl, aryl, cycloalkyl, heteroaryl or heterocycloalkyl;
R^{b} and R^{b′} are each, independently, H, C₁₋₆ alkyl, C₁₋₆ haloalkyl, C₂₋₆ alkenyl, C₂₋₆ alkynyl, aryl, cycloalkyl, heteroaryl or heterocycloalkyl;
R^{c} and R^{d} are each, independently, H, C₁₋₆ alkyl, C₁₋₆ haloalkyl, C₂₋₆ alkenyl, C₂₋₆ alkynyl, aryl, cycloalkyl, arylalkyl, or cycloalkylalkyl;
or R^{c} and R^{d} together with the N atom to which they are attached form a 4-, 5-, 6- or 7-membered heterocycloalkyl group;
R^{c′} and R^{d′} are each, independently, H, C₁₋₆ alkyl, C₁₋₆ haloalkyl, C₂₋₆ alkenyl, C₂₋₆ alkynyl, aryl, cycloalkyl, arylalkyl, or cycloalkylalkyl;
or R^{c′} and R^{d′} together with the N atom to which they are attached form a 4-, 5-, 6- or 7-membered heterocycloalkyl group;
R^{e} and R^{f} are each, independently, H, C₁₋₆ alkyl, C₁₋₆ haloalkyl, C₂₋₆ alkenyl, C₂₋₆ alkynyl, aryl, cycloalkyl, arylalkyl, or cycloalkylalkyl;
or R^{e} and R^{f} together with the N atom to which they are attached form a 4-, 5-, 6- or 7-membered heterocycloalkyl group;
m is 1, 2, 3 or 4;
n is 0, 1, 2, or 3;
t is 2 or 3;
s is 1 or 2
p is 1, 2, 3, 4 or 5;
q1 is 0, 1, 2, 3 or 4;
q2 is 0, 1, 2 or 3;
q3 is 1, 2 or 3;
q is 0, 1, 2, 3, 4 or 5; and
r is 1 or 2;
wherein when L is absent and R⁴ is (CR^{4a}R^{4b})ₜCy³, then at least one of R^{4a} and R^{4b} is other than H.

31. The compound of claim 30 wherein R³ is H or cyclopropyl

32. A compound selected from:
N-cyclohexyl-1-(phenylthio)cyclopropanecarboxamide;
1-(phenylthio)-N-[(1S)-1,2,3,4-tetrahydronaphthalen-1-yl]cyclopropanecarboxamide;
1-(phenylthio)-N-[(1R)-1,2,3,4-tetrahydronaphthalen-1-yl]cyclopropanecarboxamide;
N-[(1R,2R)-2-hydroxycyclohexyl]-1-(phenylthio)cyclopropanecarboxamide;
1-(phenylthio)-N-(tetrahydrofuran-3-yl)cyclopropanecarboxamide;
N-(2-phenylcyclopropyl)-1-(phenylthio)cyclopropanecarboxamide;
N-[(1S)-1-cyclohexylethyl]-1-(phenylthio)cyclopropanecarboxamide;
N-(1-methyl-3-phenylpropyl)-1-(phenylthio)cyclopropanecarboxamide;
N-[1-(3-hydroxy-4-methylbenzyl)propyl]-1-(phenylthio)cyclopropanecarboxamide;
N-(1,1-dimethyl-2-phenylethyl)-1-(phenylthio)cyclopropanecarboxamide;
N-[1-(hydroxymethyl)cyclopentyl]-1-(phenylthio)cyclopropanecarboxamide;
N-[(1R)-1-benzyl-2-hydroxyethyl]-1-(phenylthio)cyclopropanecarboxamide
N-[3-(hydroxymethyl)bicyclo[2.2.1]hept-2-yl]-1-(phenylthio)cyclopropanecarboxamide;
N-[(1R,2S)-2-hydroxy-2,3-dihydro-1H-inden-1-yl]-1-(phenylthio)-cyclopropanecarboxamide;
N-[(1S,2R)-2-hydroxy-1-methyl-2-phenylethyl]-1-(phenylthio)cyclopropanecarboxamide;
N-[(1S)-1-benzyl-2-methoxyethyl]-1-(phenylthio)cyclopropanecarboxamide;
N-[(1S)-2-hydroxy-1-(1H-indol-3-ylmethyl)ethyl]-1-(phenylthio)-cyclopropanecarboxamide;
N-[2-(4-chlorophenyl)-1-methylethyl]-1-(phenylthio)cyclopropanecarboxamide;
N-(2,3-dihydro-1,4-benzodioxin-2-ylmethyl)-1-(phenylthio)cyclopropanecarboxamide;
N-(3-hydroxy-2,2-dimethylpropyl)-1-(phenylthio)cyclopropanecarboxamide;
N-{[(2R)-2-hydroxycyclohexyl]methyl}-1-(phenylthio)cyclopropanecarboxamide;
N-cyclohexyl-1-[(4-fluorophenyl)thio]cyclopropanecarboxamide;
N-cyclohexyl-1-[(2,6-dichlorophenyl)thio]cyclopropanecarboxamide;
N-cyclohexyl-1-[(4′-fluorobiphenyl-4-yl)thio]cyclopropanecarboxamide;
N-cyclohexyl-1-[(3,5-dichlorophenyl)thio]cyclopropanecarboxamide;
1-[(3-Chloro-4-fluorophenyl)thio]-N-cyclohexylcyclopropanecarboxamide;
N-cyclohexyl-1-[(3,4-dichlorophenyl)thio]cyclopropanecarboxamide;
N-cyclohexyl-1-{[3-(trifluoromethyl)phenyl]thio}cyclopropanecarboxamide;
N-cyclohexyl-1-{[4-(trifluoromethoxy)phenyl]thio}cyclopropanecarboxamide;
N-cyclohexyl-1-[(2,3-dichlorophenyl)thio]cyclopropanecarboxamide;
N-cyclohexyl-1-[(2,5-dichlorophenyl)thio]cyclopropanecarboxamide;
1-[(4-chlorophenyl)thio]-N-(4-hydroxycyclohexyl)cyclopropanecarboxamide;
1-[(2-chloro-4-fluorophenyl)thio]-N-cyclohexylcyclopropanecarboxamide;
1-[(4-chlorophenyl)thio]-N-(cyclohexylmethyl)cyclopropanecarboxamide;
1-[(4-chlorophenyl)thio]-N-cyclohexylcyclopropanecarboxamide;
N-cyclohexyl-1-{[4-(2-furyl)phenyl]thio}cyclopropanecarboxamide;
N-cyclohexyl-1-(cyclohexylsulfonyl)cyclopropanecarboxamide;
N-{(3S)-1-[(3-chloro-2-methylphenyl)sulfonyl]piperidin-3-yl}-1-(phenylthio) cyclopropanecarboxamide;
N-[(3R)-1-(phenylsulfonyl)pyrrolidin-3-yl]-1-(phenylthio)cyclopropanecarboxamide;
1-[(2-chlorobenzyl)thio]-N-[(3R)-1-(phenylsulfonyl)pyrrolidin-3-yl]cyclopropane carboxamide;
N-[(3S)-1-(phenylsulfonyl)pyrrolidin-3-yl]-1-(phenylthio)cyclopropanecarboxamide;
1-[(2-chlorobenzyl)thio]-N-[(3S)-1-(phenylsulfonyl)pyrrolidin-3-yl]cyclopropane carboxamide;
1-(benzyloxy)-N-cyclohexyl-N-cyclopropylcyclopropanecarboxamide;
1-[(4-chlorobenzyl)oxy]-N-cyclohexyl-N-cyclopropylcyclopropanecarboxamide;
N-cyclohexyl-N-cyclopropyl-1-(pyridin-2-ylmethoxy)cyclopropanecarboxamide;
1-[(4-chlorophenyl)thio]-N-cyclohexyl-N-cyclopropylcyclopropanecarboxamide; and
N-cyclohexyl-1-(cyclohexylsulfonyl)-N-cyclopropylcyclopropanecarboxamide, or pharmaceutically acceptable salt form thereof.

33. A compound selected from:
1-(4-chlorophenyl)-N-cyclohexyl-N-cyclopropylcyclopropanecarboxamide;
1-(4-chlorophenyl)-N-cyclohexylcyclopropanecarboxamide;
ethyl 4-({[1-(4-chlorophenyl)cyclopropyl]carbonyl}amino)piperidine-1-carboxylate;
N-(1-benzylpiperidin-4-yl)-1-(4-chlorophenyl)cyclopropanecarboxamide;
1-(4-chlorophenyl)-N-(4-hydroxycyclohexyl)cyclopropanecarboxamide;
1-(4-chlorophenyl)-N-[(1S)-1,2,3,4-tetrahydronaphthalen-1-yl]cyclopropanecarboxamide;
1-(4-chlorophenyl)-N-[(1R)-1,2,3,4-tetrahydronaphthalen-1-yl]cyclopropanecarboxamide;
1-(4-chlorophenyl)-N-[(1R,2R)-2-hydroxycyclohexyl]cyclopropanecarboxamide;
N-[(1R,2R)-2-(benzyloxy)cyclohexyl]-1-(4-chlorophenyl)cyclopropanecarboxamide;
1-(4-chlorophenyl)-N-(tetrahydrofuran-3-yl)cyclopropanecarboxamide;
N-[(3S)-1-benzylpyrrolidin-3-yl]-1-(4-chlorophenyl)cyclopropanecarboxamide;
N-[(1R,2R)-2-(benzyloxy)cyclopentyl]-1-(4-chlorophenyl)cyclopropanecarboxamide;
N-[(1S,2S)-2-(benzyloxy)cyclopentyl]-1-(4-chlorophenyl)cyclopropanecarboxamide;
1-(4-chlorophenyl)-N-(2-phenylcyclopropyl)cyclopropanecarboxamide;
1-(4-chlorophenyl)-N-[1-(3-hydroxy-4-methylbenzyl)propyl]cyclopropanecarboxamide;
1-(4-chlorophenyl)-N-[(1R)-1-cyclohexylethyl]cyclopropanecarboxamide;
1-(4-chlorophenyl)-N-[(1S)-1-cyclohexylethyl]cyclopropanecarboxamide;
1-(4-chlorophenyl)-N-(1,1-dimethylpropyl)cyclopropanecarboxamide;
1-(4-chlorophenyl)-N-[(3S)-2-oxotetrahydrofuran-3-yl]cyclopropanecarboxamide;
1-(4-chlorophenyl)-N-(1-methyl-3-phenylpropyl)cyclopropanecarboxamide;
1-(4-chlorophenyl)-N-[(1R)-1-(hydroxymethyl)-3-methylbutyl]-cyclopropanecarboxamide;
1-(4-chlorophenyl)-N-[(1S)-1-(hydroxymethyl)-3-methylbutyl]-cyclopropanecarboxamide;
1-(4-chlorophenyl)-N-[(1R)-1-(hydroxymethyl)-2-methylpropyl]-cyclopropanecarboxamide;
1-(4-chlorophenyl)-N-[1-(hydroxymethyl)cyclopentyl]-cyclopropanecarboxamide;
N-[(1R)-1-benzyl-2-hydroxyethyl]-1-(4-chlorophenyl)cyclopropanecarboxamide;
N-[(1S)-2-(benzyloxy)-1-(hydroxymethyl)ethyl]-1-(4-chlorophenyl)-cyclopropanecarboxamide;
1-(4-chlorophenyl)-N-[(1R,2S)-2-hydroxy-2,3-dihydro-1H-inden-1-yl]-cyclopropanecarboxamide;
1-(4-chlorophenyl)-N-[(1R)-2-hydroxy-1-(4-hydroxybenzyl)ethyl]-cyclopropanecarboxamide;
1-(4-chlorophenyl)-N-[(1S,2R)-2-hydroxy-1-methyl-2-phenylethyl]-cyclopropanecarboxamide;
N-[(1S)-1-benzyl-2-methoxyethyl]-1-(4-chlorophenyl)cyclopropanecarboxamide;
1-(4-chlorophenyl)-N-[(1S)-2-cyclohexyl-1-(hydroxymethyl)ethyl]-cyclopropanecarboxamide;
1-(4-chlorophenyl)-N-[(1S)-2-hydroxy-1-(1H-indol-3-ylmethyl)ethyl]-cyclopropanecarboxamide;
N-[1-(4-chlorobenzyl)-2-hydroxyethyl]-1-(4-chlorophenyl)cyclopropanecarboxamide;
1-(4-chlorophenyl)-N-[(1S,2S)-2-hydroxycyclopentyl]cyclopropanecarboxamide;
1-(4-chlorophenyl)-N-[(1R,2S)-2-hydroxy-1-methyl-2-phenylethyl]-cyclopropanecarboxamide;
1-(4-chlorophenyl)-N-[(1S,2S)-2-hydroxy-1-(hydroxymethyl)-2-phenylethyl]-cyclopropanecarboxamide;
1-(4-chlorophenyl)-N-[(1S,2S)-2-hydroxy-1-(methoxymethyl)-2-phenylethyl]-cyclopropanecarboxamide;
1-(4-chlorophenyl)-N-(1,1-dimethyl-2-phenylethyl)cyclopropanecarboxamide;
1-(4-chlorophenyl)-N-[2-(4-chlorophenyl)-1-methylethyl]cyclopropanecarboxamide;
1-(4-chlorophenyl)-N-(2,3-dihydro-1,4-benzodioxin-2-ylmethyl)-cyclopropanecarboxamide;
ethyl 3-({[1-(4-chlorophenyl)cyclopropyl]carbonyl}amino)butanoate;
ethyl (cis)2-({[1-(4-chlorophenyl)cyclopropyl]carbonyl}amino)cyclohexanecarboxylate;
ethyl (trans)-2-({[1-(4-chlorophenyl)-cyclopropyl]carbonyl}amino)-cyclohexanecarboxylate;
methyl 4-(4-{1-[(cyclohexylamino)carbonyl]cyclopropyl} -3-fluorophenyl)piperazine-1-carboxylate;
methyl 4-(4-{1-[(1-adamantylamino)carbonyl]cyclopropyl}-3-fluorophenyl)piperazine-1-carboxylate;
methyl 4-[3-fluoro-4-(1-{[(trans-4-hydroxycyclohexyl)amino]carbonyl}cyclopropyl) phenyl]piperazine-1-carboxylate;
methyl 4-[4-(1-{[cyclohexyl(cyclopropyl)amino]carbonyl}cyclopropyl)-3-fluorophenyl]piperazine-1-carboxylate;
N-{1-[(3-Chloro-2-methylphenyl)sulfonyl]piperidin-3-yI}-1-phenylcyclopropane carboxamide;
1-(4-methoxyphenyl)-N-[(3R)-1-(phenylsulfonyl)pyrrolidin-3-yl]cyclopropane carboxamide;
1-(4-methoxyphenyl)-N-[(3 S)-1-(phenylsulfonyl)pyrrolidin-3-yl]cyclopropane carboxamide;
N-{(3S)-1-[(3-chloro-2-methylphenyl)sulfonyl]piperidin-3-yl}-1-(4-methoxyphenyl) cyclopropanecarboxamide;
1-(4-Chlorophenyl)-N-[(1S)-I -phenylethyl]cyclopropanecarboxamide;
1-(4-Chlorophenyl)-N-[(1R)-1-phenylethyl]cyclopropanecarboxamide;
1-(4-Chlorophenyl)-N-[(1R)-2-hydroxy-1-phenylethyl]cyclopropanecarboxamide;
1-(4-Chlorophenyl)-N-[(4S)-2-(hydroxymethyl)-4-phenylcyclohexylcyclopropane carboxamide;
1-(4-Chlorophenyl)-N-[3-(hydroxymethyl)bicyclo[2.2.1]hept-2-yl]cyclopropane carboxamide;
1-(4-Chlorophenyl)-N-(2-phenylethyl)cyclopropanecarboxamide;
1-(4-Chlorophenyl)-N-(2-pyridin-4-ylethyl)cyclopropanecarboxamide;
1-(4-Chlorophenyl)-N-(2-pyridin-3-ylethyl)cyclopropanecarboxamide;
1-(4-Chlorophenyl)-N-(2-pyridin-2-ylethyl)cyclopropanecarboxamide;
1-(4-Chlorophenyl)-N-(3-phenylpropyl)cyclopropanecarboxamide;
1-(4-Chlorophenyl)-N-[2-(4-hydroxyphenyl)ethyl]cyclopropanecarboxamide;
1-(4-Chlorophenyl)-N-[2-(2,4-dichlorophenyl)ethyl]cyclopropanecarboxamide;
1-(4-Chlorophenyl)-N-(2-phenoxyethyl)cyclopropanecarboxamide;
1-(4-Chlorophenyl)-N-(3-hydroxy-2,2-dimethylpropyl)cyclopropanecarboxamide;
1-(4-Chlorophenyl)-N-(2-hydroxy-3-phenoxypropyl)cyclopropanecarboxamide;
1-(4-Chlorophenyl)-N-{[(2R)-2-hydroxycyclohexyl]methyl}cyclopropanecarboxamide;
1-(4-Chlorophenyl)-N-[(2R)-2-hydroxy-2-phenylethyl]cyclopropanecarboxamide;
1-(4-Chlorophenyl)-N-(pyridin-4-ylmethyl)cyclopropanecarboxamide;
1-(4-Chlorophenyl)-N-[(3R)-1-(phenylsulfonyl)pyrrolidin-3-yl]cyclopropanecarboxamide;
1-(4-Chlorophenyl)-N-[(3S)-1-(phenylsulfonyl)pyrrolidin-3-yl]cyclopropanecarboxamide;
1-(4-Chlorophenyl)-N-[(3S)-1-(phenylsulfonyl)piperidin-3-yl]cyclopropanecarboxamide;
N-cyclopropyl-N-(cyclopropylmethyl)-1-phenylcyclopropanecarboxamide;
N-cyclopentyl-N-cyclopropyl-1-phenylcyclopropanecarboxamide;
1-(4-Chlorophenyl)-N-cyclopentyl-N-cyclopropylcyclopropanecarboxamide;
1-(4-Chlorophenyl)-N-cyclohexyl-N-cyclopropylcyclopropanecarboxamide;
1-(4-Chlorophenyl)-N-cyclopropyl-N-(tetrahydro-2H-pyran-4-yl)cyclopropane carboxamide;
tert-Butyl 4-[{[1-(4-chlorophenyl)cyclopropyl]carbonyl}(cyclopropyl)amino]piperidine-1-carboxylate;
1-(4-Chlorophenyl)-N-cyclopropyl-N-(1-methylpiperidin-4-yl)cyclopropanecarboxamide;
1-(4-Chlorophenyl)-N-cyclopropyl-N-piperidin-4-ylcyclopropanecarboxamide trifluoroacetate;
N-(1-acetylpiperidin-4-yl)-1-(4-chlorophenyl)-N-cyclopropylcyclopropanecarboxamide;
1-(4-chlorophenyl)-N-cyclopropyl-N-[1-(methylsulfonyl)piperidin-4-yl]cyclopropanecarboxamide;
methyl 4-(4-{1-[(cycloheptylamino)carbonyl]cyclopropyl}-3-fluorophenyl) piperazine-1-carboxylate;
N-cycloheptyl-1-{4-[4-(cyclopropylcarbonyl)piperazin-1-yl]-2-fluorophenyl}cyclopropanecarboxamide;
N-cycloheptyl-1-[2-fluoro-4-(4-isobutyrylpiperazin-1-yl)phenyl]cyclopropane carboxamide;
ethyl 4-[3-fluoro-4-(1-{[(trans-4-hydroxycyclohexyl)amino]carbonyl}cyclopropyl) phenyl]piperazine-1-carboxylate;
methyl 4-[3-fluoro-4-(1-{[(trans-4-hydroxycyclohexyl)(methyl)amino]carbonyl}cyclopropyl)phenyl]piperazine-1-carboxylate;
ethyl 4-[3-fluoro-4-(1-{[(trans-4-hydroxycyclohexyl)(methyl)amino]carbonyl}cyclopropyl)phenyl]piperazine-1-carboxylate;
ethyl 4-[3-fluoro-4-(1-{[(trans-4-hydroxycyclohexyl)amino]carbonyl}cyclopropyl) phenyl]piperazine-1-carboxylate;
methyl 4-[4-(1-{[(trans-4-hydroxycyclohexyl)amino]carbonyl}cyclopropyl) phenyl]piperazine-1-carboxylate;
ethyl 4-[4-(1-{[(trans-4-hydroxycyclohexyl)amino]carbonyl}cyclopropyl)phenyl]piperazine-1-carboxylate;
methyl 4-[4-(1-{[cyclohexyl(cyclopropyl)amino]carbonyl}cyclopropyl)-3-fluorophenyl]piperazine-1-carboxylate;
methyl 4-[4-(1-{[cyclohexyl(methyl)amino]carbonyl}cyclopropyl)-3-fluorophenyl]piperazine-1-carboxylate;
methyl 4-[4-(1-{[cyclohexyl(methyl)amino]carbonyl}cyclopropyl)phenyl]piperazine-1-carboxylate;
5-(4-{1-[(cyclohexylamino)carbonyl]cyclopropyl}-3-fluorophenyl)-N-ethylpyridine-2-carboxamide;
N-ethyl-5-[3-fluoro-4-(1-{[(trans-4-hydroxycyclohexyl)amino]carbonyl}cyclopropyl) phenyl]pyridine-2-carboxamide;
5-(4-{1-[(cycloheptylamino)carbonyl]cyclopropyl}-3-fluorophenyl)-N-ethylpyridine-2-carboxamide;
5-[4-(1-{[cyclohexyl(methyl)amino]carbonyl}cyclopropyl)-3-fluorophenyl]-N-ethylpyridine-2-carboxamide;
N-ethyl-5-[3-fluoro-4-(1-{[methyl(phenyl)amino]carbonyl}cyclopropyl)phenyl]pyridine-2-carboxamide;
5-[3-fluoro-4-(1-{[(trans-4-hydroxycyclohexyl)(methyl)amino]carbonyl}cyclopropyl) phenyl]-N-methylpyridine-2-carboxamide; and
5-[3-fluoro-4-(1-{[(4-hydroxy-4-methylcyclohexyl)amino]carbonyl}cyclopropyl)phenyl]-N-methylpyridine-2-carboxamide, or pharmaceutically acceptable salt form thereof.

34. A composition comprising a compound of claim 1, 18, or 32 and a pharmaceutically acceptable carrier.

35. A method of modulating 11βHSD1 or MR comprising contacting said 11βHSD1 or MR with a compound of Formula I: 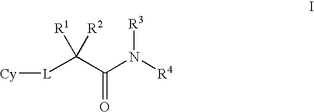
or pharmaceutically acceptable salt or prodrug thereof, wherein:
Cy is aryl, heteroaryl, cycloalkyl or heterocycloalkyl, each optionally substituted by 1, 2, 3, 4 or 5-W—X—Y-Z;
L is absent, SO₂, (CR⁶R⁷)ₘ, (CR⁶R⁷)ₙO(CR⁶R⁷)ₚ or (CR⁶R⁷)ₙS(CR⁶R⁷)ₚ;
R¹ and R² together with the C atom to which they are attached form a 3-, 4-, 5-, 6- or 7-membered cycloalkyl group or a 3-, 4-, 5-, 6- or 7-membered heterocycloalkyl group, each optionally substituted by 1, 2 or 3 R⁵;
R³ is H, C₁₋₆ alkyl, cycloalkyl, heterocycloalkyl, arylalkyl, cycloalkylalkyl, heteroarylalkyl, or heterocycloalkylalkyl;
R⁴ is C₁₋₆ alkyl, cycloalkyl, heterocycloalkyl, arylalkyl, cycloalkylalkyl, heteroarylalkyl, heterocycloalkylalkyl, each optionally substituted by 1, 2 or 3-W′—X′—Y′-Z′;
R⁵ is halo, C₁₋₄ alkyl, C₁₋₄ haloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, or NR^{c}C(O)OR^{a};
R⁶ and R⁷ are each, independently, H, halo, C₁₋₄ alkyl, C₁₋₄ haloalkyl, aryl, cycloalkyl, heteroaryl, heterocycloalkyl, CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}, NR^{c′}R^{d′}, NR^{c′}C(O)R^{d′}, NR^{c′}C(O)OR^{a′}, S(O)R^{b′}, S(O)NR^{c′}R, S(O)₂R^{b′}, or S(O)₂NR^{c′}R^{d′};
W, W′ and W″ are each, independently, absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, or NR^{e}CONR^{f}, wherein said C₁₋₆ alkylenyl, C₂₋₆ alkenylenyl, C₂₋₆ alkynylenyl are each optionally substituted by 1, 2 or 3 halo, OH, C₁₋₄ alkoxy, C₁₋₄ haloalkoxy, amino, C₁₋₄ alkylamino or C₂₋₈ dialkylamino;
X, X′ and X″ are each, independently, absent, C₁₋₆ alkylenyl, C₂₋₆ alkenylenyl, C₂₋₆ alkynylenyl, aryl, cycloalkyl, heteroaryl or heterocycloalkyl, wherein said C₁₋₆ alkylenyl, C₂₋₆ alkenylenyl, C₂₋₆ alkynylenyl, cycloalkyl, heteroaryl or heterocycloalkyl is optionally substituted by one or more halo, CN, NO₂, OH, C₁₋₄ alkoxy, C₁₋₄ haloalkoxy, amino, C₁₋₄ alkylamino or C₂₋₈ dialkylamino;
Y, Y′ and Y″ are each, independently, absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, or NR^{e}CONR^{f}, wherein said C₁₋₆ alkylenyl, C₂₋₆ alkenylenyl, C₂₋₆ alkynylenyl are each optionally substituted by 1, 2 or 3 halo, OH, C₁₋₄ alkoxy, C₁₋₄ haloalkoxy, amino, C₁₋₄ alkylamino or C₂₋₈ dialkylamino;
Z, Z′ and Z″ are each, independently, H, halo, CN, NO₂, OH, C₁₋₄ alkoxy, C₁₋₄ haloalkoxy, amino, C₁₋₄ alkylamino or C₂₋₈ dialkylamino, C₁₋₆ alkyl, C₂₋₆ alkenyl, C₂₋₆ alkynyl, aryl, cycloalkyl, heteroaryl or heterocycloalkyl, wherein said C₁₋₆ alkyl, C₂₋₆ alkenyl, C₂₋₆ alkynyl, aryl, cycloalkyl, heteroaryl or heterocycloalkyl is optionally substituted by 1, 2 or 3 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, S(O)R^{b}, S(O)NR^{c}R^{d}, S(O)₂R^{b}, or S(O)₂NR^{c}R^{d};
wherein two —W—X—Y-Z attached to the same atom, together with the atom to which they are attached, optionally form a 3-20 membered cycloalkyl or heterocycloalkyl group each optionally substituted by 1, 2 or 3-W″—X″—Y″-Z″;
or wherein two —W—X—Y-Z together with the carbon atom to which they are both attached optionally form a carbonyl;
or wherein two —W—X—Y-Z together with two adjacent atoms to which they are attached optionally form a 3-20 membered fused cycloalkyl group or 3-20 membered fused heterocycloalkyl group, each optionally substituted by 1, 2 or 3-W″—X″—Y″-Z″;
wherein two —W′—X′—Y′-Z′ together with the atom to which they are both attached optionally form a 3-20 membered cycloalkyl group or 3-20 membered heterocycloalkyl group, each optionally substituted by 1, 2 or 3-W″—X″—Y″-Z″;
or wherein two —W′—X′—Y′-Z′ together with the carbon atom to which they are both attached optionally form a carbonyl;
or wherein two —W′—X′—Y′-Z′ together with two adjacent atoms to which they are attached optionally form a 3-20 membered fused cycloalkyl group or 3-20 membered fused heterocycloalkyl group, each optionally substituted by 1, 2 or 3-W″—X″—Y″-Z″;
or wherein two —W′—X′—Y′-Z′ together with two adjacent atoms to which they are attached optionally form a 5- or 6-membered fused aryl or 5- or 6-membered fused heteroaryl group, each optionally substituted by 1, 2 or 3-W″—X″—Y″-Z″;
wherein —W—X—Y-Z is other than H;
wherein —W′—X′—Y′-Z′ is other than H;
wherein —W″—X″—Y″-Z″ is other than H;
R^{a} and R^{a′} are each, independently, H, C₁₋₆ alkyl, C₁₋₆ haloalkyl, C₂₋₆ alkenyl, C₂₋₆ alkynyl, aryl, cycloalkyl, heteroaryl or heterocycloalkyl;
R^{b} and R^{b′} are each, independently, H, C₁₋₆ alkyl, C₁₋₆ haloalkyl, C₂₋₆ alkenyl, C₂₋₆ alkynyl, aryl, cycloalkyl, heteroaryl or heterocycloalkyl;
R^{c} and R^{d} are each, independently, H, C₁₋₆ alkyl, C₁₋₆ haloalkyl, C₂₋₆ alkenyl, C₂₋₆ alkynyl, cycloalkyl, arylalkyl, or cycloalkylalkyl;
or R^{c} and R^{d} together with the N atom to which they are attached form a 4-, 5-, 6- or 7-membered heterocycloalkyl group;
R^{c′} and R^{d′} are each, independently, H, C₁₋₆ alkyl, C₁₋₆ haloalkyl, C₂₋₆ alkenyl, C₂₋₆ alkynyl, aryl, cycloalkyl, arylalkyl, or cycloalkylalkyl;
or R^{c′} and R^{d′} together with the N atom to which they are attached form a 4-, 5-, 6- or 7-membered heterocycloalkyl group;
R^{e} and R^{f} are each, independently, H, C₁₋₆ alkyl, C₁₋₆ haloalkyl, C₂₋₆ alkenyl, C₂₋₆ alkynyl, aryl, cycloalkyl, arylalkyl, or cycloalkylalkyl;
or R^{e} and R^{f} together with the N atom to which they are attached form a 4-, 5-, 6- or 7-membered heterocycloalkyl group;
m is 1, 2, 3 or 4;
n is 0, 1, 2 or 3; and
p is 0, 1, 2 or 3.

36. The method of claim 35 wherein said modulating is inhibiting.

37. A method of inhibiting conversion of cortisone to cortisol in a cell comprising contacting said cell with a compound of Formula I: 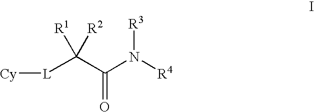
or pharmaceutically acceptable salt or prodrug thereof, wherein:
Cy is aryl, heteroaryl, cycloalkyl or heterocycloalkyl, each optionally substituted by 1, 2, 3, 4 or 5-W—X—Y-Z;
L is absent, SO₂, (CR⁶R⁷)ₘ, (CR⁶R⁷)ₙO(CR⁶R⁷)ₚ or (CR⁶R⁷)ₙS(CR⁶R⁷)ₚ;
R¹ and R² together with the C atom to which they are attached form a 3-, 4-, 5-, 6- or 7-membered cycloalkyl group or a 3-, 4-, 5-, 6- or 7-membered heterocycloalkyl group, each optionally substituted by 1, 2 or 3 R⁵;
R³ is H, C₁₋₆ alkyl, cycloalkyl, heterocycloalkyl, arylalkyl, cycloalkylalkyl, heteroarylalkyl, or heterocycloalkylalkyl;
R⁴ is C₁₋₆ alkyl, cycloalkyl, heterocycloalkyl, arylalkyl, cycloalkylalkyl, heteroarylalkyl, heterocycloalkylalkyl, each optionally substituted by 1, 2 or 3-W′—X′—Y′-Z′;
R⁵ is halo, C₁₋₄ alkyl, C₁₋₄ haloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, or NR^{c}C(O)OR^{a};
R⁶ and R⁷ are each, independently, H, halo, C₁₋₄ alkyl, C₁₋₄ haloalkyl, aryl, cycloalkyl, heteroaryl, heterocycloalkyl, CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}, NR^{c′}R^{d′}, NR^{c′}C(O)R^{d′}, NR^{c′}C(O)OR^{a′}, S(O)R^{b′}, S(O)NR^{c′}R^{d′}, S(O)₂R^{b′}, or S(O)₂NR^{c′}R^{d′};
W, W′ and W″ are each, independently, absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, or NR^{e}CONR^{f}, wherein said C₁₋₆ alkylenyl, C₂₋₆ alkenylenyl, C₂₋₆ alkynylenyl are each optionally substituted by 1, 2 or 3 halo, OH, C₁₋₄ alkoxy, C₁₋₄ haloalkoxy, amino, C₁₋₄ alkylamino or C₂₋₈ dialkylamino;
X, X′ and X″ are each, independently, absent, C₁₋₄ alkylenyl, C₂₋₆ alkenylenyl, C₂₋₆ alkynylenyl, aryl, cycloalkyl, heteroaryl or heterocycloalkyl, wherein said C₁₋₆ alkylenyl, C₂₋₆ alkenylenyl, C₂₋₆ alkynylenyl, cycloalkyl, heteroaryl or heterocycloalkyl is optionally substituted by one or more halo, CN, NO₂, OH, C₁₋₄ alkoxy, C₁₋₄ haloalkoxy, amino, C₁₋₄ alkylamino or C₂₋₈ dialkylamino;
Y, Y′ and Y″ are each, independently, absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, or NR^{e}CONR^{f}, wherein said C₁₋₆ alkylenyl, C₂₋₆ alkenylenyl, C₂₋₆ alkynylenyl are each optionally substituted by 1, 2 or 3 halo, OH, C₁₋₄ alkoxy, C₁₋₄ haloalkoxy, amino, C₁₋₄ alkylamino or C₂₋₈ dialkylamino;
Z, Z′ and Z″ are each, independently, H, halo, CN, NO₂, OH, C₁₋₄ alkoxy, C₁₋₄ haloalkoxy, amino, C₁₋₄ alkylamino or C₂₋₈ dialkylamino, C₁₋₆ alkyl, C₂₋₆ alkenyl, C₂₋₆ alkynyl, aryl, heteroaryl or heterocycloalkyl, wherein said C₁₋₆ alkyl, C₂₋₆ alkenyl, C₂₋₆ alkynyl, aryl, cycloalkyl, heteroaryl or heterocycloalkyl is optionally substituted by 1, 2 or 3 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, S(O)R^{b}, S(O)NR^{c}R^{d}, S(O)₂R^{b}, or S(O)₂NR^{c}R^{d};
wherein two —W—X—Y-Z attached to the same atom, together with the atom to which they are attached, optionally form a 3-20 membered cycloalkyl or heterocycloalkyl group each optionally substituted by 1, 2 or 3-W″—X″—Y″-Z″;
or wherein two —W—X—Y-Z together with the carbon atom to which they are both attached optionally form a carbonyl;
or wherein two —W—X—Y-Z together with two adjacent atoms to which they are attached optionally form a 3-20 membered fused cycloalkyl group or 3-20 membered fused heterocycloalkyl group, each optionally substituted by 1, 2 or 3-W″—X″—Y″-Z″;
wherein two —W′—X′—Y′-Z′ together with the atom to which they are both attached optionally form a 3-20 membered cycloalkyl group or 3-20 membered heterocycloalkyl group, each optionally substituted by 1, 2 or 3-W″—X″—Y″-Z″;
or wherein two —W′—X′—Y′-Z′ together with the carbon atom to which they are both attached optionally form a carbonyl;
or wherein two —W′—X′—Y′-Z′ together with two adjacent atoms to which they are attached optionally form a 3-20 membered fused cycloalkyl group or 3-20 membered fused heterocycloalkyl group, each optionally substituted by 1, 2 or 3-W″—X″—Y″-Z″;
or wherein two —W′—X′—Y′-Z′ together with two adjacent atoms to which they are attached optionally form a 5- or 6-membered fused aryl or 5- or 6-membered fused heteroaryl group, each optionally substituted by 1, 2 or 3-W″—X″—Y″-Z″;
wherein —W—X—Y-Z is other than H;
wherein —W′—X′—Y′-Z′ is other than H;
wherein —W″—X″—Y″-Z″ is other than H;
R^{a} and R^{a′} are each, independently, H, C₁₋₆ alkyl, C₁₋₆ haloalkyl, C₂₋₆ alkenyl, C₂₋₆ alkynyl, aryl, cycloalkyl, heteroaryl or heterocycloalkyl;
R^{b} and R^{b′} are each, independently, H, C₁₋₆ alkyl, C₁₋₆ haloalkyl, C₂₋₆ alkenyl, C₂₋₆ alkynyl, aryl, cycloalkyl, heteroaryl or heterocycloalkyl;
R^{c} and R^{d} are each, independently, H, C₁₋₆ alkyl, C₁₋₆ haloalkyl, C₂₋₆ alkenyl, C₂₋₆ alkynyl, aryl, cycloalkyl, arylalkyl, or cycloalkylalkyl;
or R^{c} and R^{d} together with the N atom to which they are attached form a 4-, 5-, 6- or 7-membered heterocycloalkyl group;
R^{c}′ and R^{d′} are each, independently, H, C₁₋₆ alkyl, C₁₋₆ haloalkyl, C₂₋₆ alkenyl, C₂₋₆ alkynyl, aryl, cycloalkyl, arylalkyl, or cycloalkylalkyl;
or R^{c′} and R^{d′} together with the N atom to which they are attached form a 4-, 5-, 6- or 7-membered heterocycloalkyl group;
R^{e} and R^{f} are each, independently, H, C₁₋₆ alkyl, C₁₋₆ haloalkyl, C₂₋₆ alkenyl, C₂₋₆ alkynyl, aryl, cycloalkyl, arylalkyl, or cycloalkylalkyl;
or R^{e} and R^{f} together with the N atom to which they are attached form a 4-, 5-, 6- or 7-membered heterocycloalkyl group;
m is 1, 2, 3 or 4;
n is 0, 1, 2 or 3; and
p is 0, 1, 2 or 3.

38. A method of inhibiting production of cortisol in a cell comprising contacting said cell with a compound of Formula I: 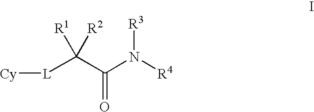
or pharmaceutically acceptable salt or prodrug thereof, wherein:
Cy is aryl, heteroaryl, cycloalkyl or heterocycloalkyl, each optionally substituted by 1, 2, 3, 4 or 5-W—X—Y-Z;
L is absent, SO₂, (CR⁶R⁷)ₘ, (CR⁶R⁷)ₙO(CR⁶R⁷)ₚ or (CR⁶R⁷), S(CR⁶R⁷)ₚ;
R¹ and R² together with the C atom to which they are attached form a 3-, 4-, 5-, 6- or 7-membered cycloalkyl group or a 3-, 4-, 5-, 6- or 7-membered heterocycloalkyl group, each optionally substituted by 1, 2 or 3 R⁵;
R³ is H, C₁₋₆ alkyl, cycloalkyl, heterocycloalkyl, arylalkyl, cycloalkylalkyl, heteroarylalkyl, or heterocycloalkylalkyl;
R⁴ is C₁₋₆ alkyl, cycloalkyl, heterocycloalkyl, arylalkyl, cycloalkylalkyl, heteroarylalkyl, heterocycloalkylalkyl, each optionally substituted by 1, 2 or 3-W′—X′—Y′-Z′;
R¹ is halo, C₁₋₄ alkyl, C₁₋₄ haloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, or NR^{c}C(O)OR^{a};
R⁶ and R⁷ are each, independently, H, halo, C₁₋₄ alkyl, C₁₋₄ haloalkyl, aryl, cycloalkyl, heteroaryl, heterocycloalkyl, CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}, NR^{c′}R^{d′}, NR^{c′}C(O)R^{d′}, NR^{c′}C(O)OR^{a′}, S(O)R^{b′}, S(O)NR^{c′}R^{d′}, S(O)₂R^{b′}, or S(O)₂NR^{c′}R^{d′};
W, W′ and W″ are each, independently, absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, or NR^{e}CONR^{f}, wherein said C₁₋₆ alkylenyl, C₂₋₆ alkenylenyl, C₂₋₆ alkynylenyl are each optionally substituted by 1, 2 or 3 halo, OH, C₁₋₄ alkoxy, C₁₋₄ haloalkoxy, amino, C₁₋₄ alkylamino or C₂₋₈ dialkylamino;
X, X′ and X″ are each, independently, absent, C₁₋₆ alkylenyl, C₂₋₆ alkenylenyl, C₂₋₆ alkynylenyl, aryl, cycloalkyl, heteroaryl or heterocycloalkyl, wherein said C₁₋₆ alkylenyl, C₂₋₆ alkenylenyl, C₂₋₆ alkynylenyl, cycloalkyl, heteroaryl or heterocycloalkyl is optionally substituted by one or more halo, CN, NO₂, OH, C₁₋₄ alkoxy, C₁₋₄ haloalkoxy, amino, C₁₋₄ alkylamino or C₂₋₈ dialkylamino;
Y, Y′ and Y″ are each, independently, absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, or NR^{e}CONR^{f}, wherein said C₁₋₆ alkylenyl, C₂₋₆ alkenylenyl, C₂₋₆ alkynylenyl are each optionally substituted by 1, 2 or 3 halo, OH, C₁₋₄ alkoxy, C₁₋₄ haloalkoxy, amino, C₁₋₄ alkylamino or C₂₋₈ dialkylamino;
Z, Z′ and Z″ are each, independently, H, halo, CN, NO₂, OH, C₁₋₄ alkoxy, C₁₋₄ haloalkoxy, amino, C₁₋₄ alkylamino or C₂₋₈ dialkylamino, C₁₋₆ alkyl, C₂₋₆ alkenyl, C₂₋₆ alkynyl, aryl, cycloalkyl, heteroaryl or heterocycloalkyl, wherein said C₁₋₆ alkyl, C₂₋₆ alkenyl, C₂₋₆ alkynyl, aryl, cycloalkyl, heteroaryl or heterocycloalkyl is optionally substituted by 1, 2 or 3 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NRCR^{d}, NR^{c}C(O)R^{d}, NR^{c}C(O)OR^{a}, S(O)R^{b}, S(O)NR^{c}R^{d}, S(O)₂R^{b}, or S(O)₂NR^{c}R^{d};
wherein two —W—X—Y-Z attached to the same atom, together with the atom to which they are attached, optionally form a 3-20 membered cycloalkyl or heterocycloalkyl group each optionally substituted by 1, 2 or 3-W″—X″—Y″-Z″;
or wherein two —W—X—Y-Z together with the carbon atom to which they are both attached optionally form a carbonyl;
or wherein two —W—X—Y-Z together with two adjacent atoms to which they are attached optionally form a 3-20 membered fused cycloalkyl group or 3-20 membered fused heterocycloalkyl group, each optionally substituted by 1, 2 or 3-W″—X″—Y″-Z″;
wherein two —W′—X′—Y′-Z′ together with the atom to which they are both attached optionally form a 3-20 membered cycloalkyl group or 3-20 membered heterocycloalkyl group, each optionally substituted by 1, 2 or 3-W″—X″—Y″-Z″;
or wherein two —W′—X′—Y′-Z′ together with the carbon atom to which they are both attached optionally form a carbonyl;
or wherein two —W′—X′—Y′-Z′ together with two adjacent atoms to which they are attached optionally form a 3-20 membered fused cycloalkyl group or 3-20 membered fused heterocycloalkyl group, each optionally substituted by 1, 2 or 3-W″—X″—Y″-Z″;
or wherein two —W′—X′—Y′-Z′ together with two adjacent atoms to which they are attached optionally form a 5- or 6-membered fused aryl or 5- or 6-membered fused heteroaryl group, each optionally substituted by 1, 2 or 3-W″—X″—Y″-Z″;
wherein —W—X—Y-Z is other than H;
wherein —W′—X′—Y′-Z′ is other than H;
wherein —W″—X″—Y″-Z″ is other than H;
R^{a} and R^{a′} are each, independently, H, C₁₋₆ alkyl, C₁₋₆ haloalkyl, C₂₋₆ alkenyl, C₂₋₆ alkynyl, aryl, cycloalkyl, heteroaryl or heterocycloalkyl;
R^{b} and R^{b′} are each, independently, H, C₁₋₆ alkyl, C₁₋₆ haloalkyl, C₂₋₆ alkenyl, C₂₋₆ alkynyl, aryl, cycloalkyl, heteroaryl or heterocycloalkyl;
R^{c} and R^{d} are each, independently, H, C₁₋₆ alkyl, C₁₋₆ haloalkyl, C₂₋₆ alkenyl, C₂₋₆ alkynyl, aryl, cycloalkyl, arylalkyl, or cycloalkylalkyl;
or R^{c} and R^{d} together with the N atom to which they are attached form a 4-, 5-, 6- or 7-membered heterocycloalkyl group;
R^{c′} and R^{d′} are each, independently, H, C₁₋₆ alkyl, C₁₋₆ haloalkyl, C₂₋₆ alkenyl, C₂₋₆ alkynyl, aryl, cycloalkyl, arylalkyl, or cycloalkylalkyl;
or R^{c′} and R^{d′} together with the N atom to which they are attached form a 4-, 5-, 6- or 7-membered heterocycloalkyl group;
R^{e} and R^{f} are each, independently, H, C₁₋₆ alkyl, C₁₋₆ haloalkyl, C₂₋₆ alkenyl, C₂₋₆ alkynyl, aryl, cycloalkyl, arylalkyl, or cycloalkylalkyl;
or R^{e} and R^{f} together with the N atom to which they are attached form a 4-, 5-, 6- or 7-membered heterocycloalkyl group;
m is 1, 2, 3 or 4;
n is 0, 1, 2 or 3; and
p is 0, 1, 2 or 3.

39. A method of increasing insulin sensitivity in a cell comprising contacting said cell with a compound of Formula I: 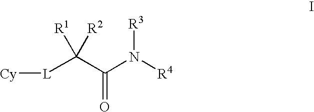
or pharmaceutically acceptable salt or prodrug thereof, wherein:
Cy is aryl, heteroaryl, cycloalkyl or heterocycloalkyl, each optionally substituted by 1, 2, 3, 4 or 5-W—X—Y-Z;
L is absent, SO₂, (CR⁶R⁷)ₘ, (CR⁶R⁷)ₙO(CR⁶R⁷)ₚ or (CR⁶R⁷)ₙS(CR⁶R⁷)ₚ;
R¹ and R² together with the C atom to which they are attached form a 3-, 4-, 5-, 6- or 7-membered cycloalkyl group or a 3-, 4-, 5-, 6- or 7-membered heterocycloalkyl group, each optionally substituted by 1, 2 or 3 R⁵;
R³ is H, C₁₋₆ alkyl, cycloalkyl, heterocycloalkyl, arylalkyl, cycloalkylalkyl, heteroarylalkyl, or heterocycloalkylalkyl;
R⁴ is C₁₋₆ alkyl, cycloalkyl, heterocycloalkyl, arylalkyl, cycloalkylalkyl, heteroarylalkyl, heterocycloalkylalkyl, each optionally substituted by 1, 2 or 3-W′—X′—Y′-Z′;
R⁵ is halo, C₁₋₄ alkyl, C₁₋₄ haloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, or NR^{c}C(O)OR^{a};
R⁶ and R⁷ are each, independently, H, halo, C₁₋₄ alkyl, C₁₋₄ haloalkyl, aryl, cycloalkyl, heteroaryl, heterocycloalkyl, CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}, NR^{c′}R^{d′}, NR^{c′}C(O)R^{d′}, NR^{c′}C(O)OR^{a′}, S(O)R^{b′}, S(O)NR^{c′}R^{d′}, S(O)₂R^{b′}, or S(O)₂NR^{c′}R^{d′};
W, W″ and W″ are each, independently, absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, or NR^{e}CONR^{f}, wherein said C₁₋₆ alkylenyl, C₂₋₆ alkenylenyl, C₂₋₆ alkynylenyl are each optionally substituted by 1, 2 or 3 halo, OH, C₁₋₄ alkoxy, C₁₋₄ haloalkoxy, amino, C₁₋₄ alkylamino or C₂₋₈ dialkylamino;
X, X′ and X″ are each, independently, absent, C₁₋₆ alkylenyl, C₂₋₆ alkenylenyl, C₂₋₆ alkynylenyl, aryl, cycloalkyl, heteroaryl or heterocycloalkyl, wherein said C₁₋₆ alkylenyl, C₂₋₆ alkenylenyl, C₂₋₆ alkynylenyl, cycloalkyl, heteroaryl or heterocycloalkyl is optionally substituted by one or more halo, CN, NO₂, OH, C₁₋₄ alkoxy, C₁₋₄ haloalkoxy, amino, C₁₋₄ alkylamino or C₂₋₈ dialkylamino;
Y, Y′ and Y″ are each, independently, absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, or NR^{e}CONR^{f}, wherein said C₁₋₆ alkylenyl, C₂₋₆ alkenylenyl, C₂₋₆ alkynylenyl are each optionally substituted by 1, 2 or 3 halo, OH, C₁₋₄ alkoxy, C₁₋₄ haloalkoxy, amino, C₁₋₄ alkylamino or C₂₋₈ dialkylamino;
Z, Z′ and Z″ are each, independently, H, halo, CN, NO₂, OH, C₁₋₄ alkoxy, C₁₋₄ haloalkoxy, amino, C₁₋₄ alkylamino or C₂₋₈ dialkylamino, C₁₋₆ alkyl, C₂₋₆ alkenyl, C₂₋₆ alkynyl, aryl, cycloalkyl, heteroaryl or heterocycloalkyl, wherein said C₁₋₆ alkyl, C₂₋₆ alkenyl, C₂₋₆ alkynyl, aryl, cycloalkyl, heteroaryl or heterocycloalkyl is optionally substituted by 1, 2 or 3 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, S(O)R^{b}, S(O)NR^{c}R^{d}, S(O)₂R^{b}, or S(O)₂NR^{c}R^{d};
wherein two —W—X—Y-Z attached to the same atom, together with the atom to which they are attached, optionally form a 3-20 membered cycloalkyl or heterocycloalkyl group each optionally substituted by 1, 2 or 3-W″—X″—Y″-Z″;
or wherein two —W—X—Y-Z together with the carbon atom to which they are both attached optionally form a carbonyl;
or wherein two —W—X—Y-Z together with two adjacent atoms to which they are attached optionally form a 3-20 membered fused cycloalkyl group or 3-20 membered fused heterocycloalkyl group, each optionally substituted by 1, 2 or 3-W″—X″—Y″-Z″;
wherein two —W′—X′—Y′-Z′ together with the atom to which they are both attached optionally form a 3-20 membered cycloalkyl group or 3-20 membered heterocycloalkyl group, each optionally substituted by 1, 2 or 3-W″—X″—Y″-Z″;
or wherein two —W′—X′—Y′-Z′ together with the carbon atom to which they are both attached optionally form a carbonyl;
or wherein two —W′—X′—Y′-Z′ together with two adjacent atoms to which they are attached optionally form a 3-20 membered fused cycloalkyl group or 3-20 membered fused heterocycloalkyl group, each optionally substituted by 1, 2 or 3-W″—X″—Y″-Z″;
or wherein two —W′—X′—Y′-Z′ together with two adjacent atoms to which they are attached optionally form a 5- or 6-membered fused aryl or 5- or 6-membered fused heteroaryl group, each optionally substituted by 1, 2 or 3-W″—X″—Y″-Z″;
wherein —W—X—Y-Z is other than H;
wherein —W′—X′—Y′-Z′ is other than H;
wherein —W″—X″—Y″-Z″ is other than H;
R^{a} and R^{a′} are each, independently, H, C₁₋₆ alkyl, C₁₋₆ haloalkyl, C₂₋₆ alkenyl, C₂₋₆ alkynyl, aryl, cycloalkyl, heteroaryl or heterocycloalkyl;
R^{b} and R^{b′} are each, independently, H, C₁₋₆ alkyl, C₁₋₆ haloalkyl, C₂₋₆ alkenyl, C₂₋₆ alkynyl, aryl, cycloalkyl, heteroaryl or heterocycloalkyl;
R^{c} and R^{d} are each, independently, H, C₁₋₆ alkyl, C₁₋₆ haloalkyl, C₂₋₆ alkenyl, C₂₋₆ alkynyl, aryl, cycloalkyl, arylalkyl, or cycloalkylalkyl;
or R^{c} and R^{d} together with the N atom to which they are attached form a 4-, 5-, 6- or 7-membered heterocycloalkyl group;
R^{c′} and R^{d′} are each, independently, H, C₁₋₆ alkyl, C₁₋₆ haloalkyl, C₂₋₆ alkenyl, C₂₋₆ alkynyl, aryl, cycloalkyl, arylalkyl, or cycloalkylalkyl;
or R^{c′} and R^{d′} together with the N atom to which they are attached form a 4-, 5-, 6- or 7-membered heterocycloalkyl group;
R^{e} and R^{f} are each, independently, H, C₁₋₆ alkyl, C₁₋₆ haloalkyl, C₂₋₆ alkenyl, C₂₋₆ alkynyl, aryl, cycloalkyl, arylalkyl, or cycloalkylalkyl;
or R^{e} and R^{f} together with the N atom to which they are attached form a 4-, 5-, 6- or 7-membered heterocycloalkyl group;
m is 1, 2, 3 or 4;
n is 0, 1, 2 or 3; and
p is 0, 1, 2 or 3.

40. A method of treating a disease in a patient, wherein said disease is associated with expression or activity of 11βHSD1 or MR, comprising administering to said patient a therapeutically effective amount of a compound of Formula I: 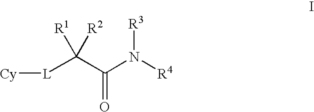
or pharmaceutically acceptable salt or prodrug thereof, wherein:
Cy is aryl, heteroaryl, cycloalkyl or heterocycloalkyl, each optionally substituted by 1, 2, 3, 4 or 5-W—X—Y-Z;
L is absent, SO₂, (CR⁶R⁷)ₘ, (CR⁶R⁷)ₙO(CR⁶R⁷)ₚ or (CR⁶R⁷)ₙS(CR⁶R⁷)ₚ;
R¹ and R² together with the C atom to which they are attached form a 3-, 4-, 5-, 6- or 7-membered cycloalkyl group or a 3-, 4-, 5-, 6- or 7-membered heterocycloalkyl group, each optionally substituted by 1, 2 or 3 R⁵;
R³ is H, C₁₋₆alkyl, cycloalkyl, heterocycloalkyl, arylalkyl, cycloalkylalkyl, heteroarylalkyl, or heterocycloalkylalkyl;
R⁴ is C₁₋₆alkyl, cycloalkyl, heterocycloalkyl, arylalkyl, cycloalkylalkyl, heteroarylalkyl, heterocycloalkylalkyl, each optionally substituted by 1, 2 or 3-W′—X′—Y′-Z′;
R⁵ is halo, C₁₋₄ alkyl, C₁₋₄ haloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, or NR^{c}C(O)OR^{a};
R⁶ and R⁷ are each, independently, H, halo, C₁₋₄ alkyl, C₁₋₄ haloalkyl, aryl, cycloalkyl, heteroaryl, heterocycloalkyl, CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}, NR^{c′}R^{d′}, NR^{c′}C(O)R^{d′}, NR^{c′}C(O)OR^{a′}, S(O)R^{b′}, S(O)NR^{c′}R^{d′}, S(O)₂R^{b′}, or S(O)₂NR^{c′}R^{d′};
W, W′ and W″ are each, independently, absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, or NR^{e}CONR^{f}, wherein said C₁₋₆ alkylenyl, C₂₋₆ alkenylenyl, C₂₋₆ alkynylenyl are each optionally substituted by 1, 2 or 3 halo, OH, C₁₋₄ alkoxy, C₁₋₄ haloalkoxy, amino, C₁₋₄ alkylamino or C₂₋₈ dialkylamino;
X, X′ and X″ are each, independently, absent, C₁₋₆ alkylenyl, C₂₋₆ alkenylenyl, C₂₋₆ alkynylenyl, aryl, cycloalkyl, heteroaryl or heterocycloalkyl, wherein said C₁₋₆ alkylenyl, C₂₋₆ alkenylenyl, C₂₋₆ alkynylenyl, cycloalkyl, heteroaryl or heterocycloalkyl is optionally substituted by one or more halo, CN, NO₂, OH, C₁₋₄ alkoxy, C₁₋₄ haloalkoxy, amino, C₁₋₄ alkylamino or C₂₋₈ dialkylamino;
Y, Y′ and Y″ are each, independently, absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, or NR^{e}CONR^{f}, wherein said C₁₋₆ alkylenyl, C₂₋₆ alkenylenyl, C₂₋₆ alkynylenyl are each optionally substituted by 1, 2 or 3 halo, OH, C₁₋₄ alkoxy, C₁₋₄ haloalkoxy, amino, C₁₋₄ alkylamino or C₂₋₈ dialkylamino;
Z, Z′ and Z″ are each, independently, H, halo, CN, NO₂, OH, C₁₋₄ alkoxy, C₁₋₄ haloalkoxy, amino, C₁₋₄ alkylamino or C₂₋₈ dialkylamino, C₁₋₆ alkyl, C₂₋₆ alkenyl, C₂₋₆ alkynyl, aryl, cycloalkyl, heteroaryl or heterocycloalkyl, wherein said C₁₋₆ alkyl, C₂₋₆ alkenyl, C₂₋₆ alkynyl, aryl, cycloalkyl, heteroaryl or heterocycloalkyl is optionally substituted by 1, 2 or 3 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, S(O)R^{b}, S(O)NR^{c}R^{d}, S(O)₂R^{b}, or S(O)₂NR^{c}R^{d};
wherein two —W—X—Y-Z attached to the same atom, together with the atom to which they are attached, optionally form a 3-20 membered cycloalkyl or heterocycloalkyl group each optionally substituted by 1, 2 or 3-W″—X″—Y″-Z″;
or wherein two —W—X—Y-Z together with the carbon atom to which they are both attached optionally form a carbonyl;
or wherein two —W—X—Y-Z together with two adjacent atoms to which they are attached optionally form a 3-20 membered fused cycloalkyl group or 3-20 membered fused heterocycloalkyl group, each optionally substituted by 1, 2 or 3-W″—X″—Y″-Z″;
wherein two —W′—X′—Y′-Z′ together with the atom to which they are both attached optionally form a 3-20 membered cycloalkyl group or 3-20 membered heterocycloalkyl group, each optionally substituted by 1, 2 or 3-W″—X″—Y″-Z″;
or wherein two —W′—X′—Y′-Z′ together with the carbon atom to which they are both attached optionally form a carbonyl;
or wherein two —W′—X′—Y′-Z′ together with two adjacent atoms to which they are attached optionally form a 3-20 membered fused cycloalkyl group or 3-20 membered fused heterocycloalkyl group, each optionally substituted by 1, 2 or 3-W″—X″—Y″-Z″;
or wherein two —W′—X′—Y′-Z′ together with two adjacent atoms to which they are attached optionally form a 5- or 6-membered fused aryl or 5- or 6-membered fused heteroaryl group, each optionally substituted by 1, 2 or 3-W″—X″—Y″-Z″;
wherein —W—X—Y-Z is other than H;
wherein —W′—X′—Y′-Z′ is other than H;
wherein —W″—X″—Y″-Z″ is other than H;
R^{a} and R^{a′} are each, independently, H, C₁₋₆ alkyl, C₁₋₆ haloalkyl, C₂₋₆ alkenyl, C₂₋₆ alkynyl, aryl, cycloalkyl, heteroaryl or heterocycloalkyl;
R^{b} and R^{b′} are each, independently, H, C₁₋₆ alkyl, C₁₋₆ haloalkyl, C₂₋₆ alkenyl, C₂₋₆ alkynyl, aryl, cycloalkyl, heteroaryl or heterocycloalkyl;
R^{c} and R^{d} are each, independently, H, C₁₋₆ alkyl, C₁₋₆ haloalkyl, C₂₋₆ alkenyl, C₂₋₆ alkynyl, aryl, cycloalkyl, arylalkyl, or cycloalkylalkyl;
or R^{c} and R^{d} together with the N atom to which they are attached form a 4-, 5-, 6- or 7-membered heterocycloalkyl group;
R^{c′} and R^{d′} are each, independently, H, C₁₋₆ alkyl, C₁₋₆ haloalkyl, C₂₋₆ alkenyl, C₂₋₆ alkynyl, aryl, cycloalkyl, arylalkyl, or cycloalkylalkyl;
or R^{c′} and R^{d′} together with the N atom to which they are attached form a 4-, 5-, 6- or 7-membered heterocycloalkyl group;
R^{e} and R^{f} are each, independently, H, C₁₋₆ alkyl, C₁₋₆ haloalkyl, C₂₋₆ alkenyl, C₂₋₆ alkynyl, aryl, cycloalkyl, arylalkyl, or cycloalkylalkyl;
or R^{e} and R^{f} together with the N atom to which they are attached form a 4-, 5-, 6- or 7-membered heterocycloalkyl group;
m is 1, 2, 3 or 4;
n is 0, 1, 2 or 3; and
p is 0, 1, 2 or 3.

41. The method of claim 40 wherein said disease is obesity, diabetes, glucose intolerance, insulin resistance, hyperglycemia, hypertension, hyperlipidemia, cognitive impairment, depression, dementia, glaucoma, cardiovascular disorders, osteoporosis, inflammation, a cardiovascular, renal or inflammatory disease, heart failure, atherosclerosis, arteriosclerosis, coronary artery disease, thrombosis, angina, peripheral vascular disease, vascular wall damage, stroke, dyslipidemia, hyperlipoproteinaemia, diabetic dyslipidemia, mixed dyslipidemia, hypercholesterolemia, hypertriglyceridemia, metabolic syndrome or general aldosterone-related target organ damage.
